(19) United States
(12) Patent Application Publication
(10) Pub. No.: US 2023/0122972 A1
(43) Pub. Date: Apr. 20, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Youngkyoung JO, Suwon-si (KR); Dong Min KANG, Suwon-si (KR); Chang Ju SHIN, Suwon-si (KR); Seungjae LEE, Suwon-si (KR); Changwoo KIM, Suwon-si (KR); Hyung Sun KIM, Suwon-si (KR); Jinhyun LUI, Suwon-si (KR); Seungin PARK, Suwon-si (KR); Jongwoo WON, Suwon-si (KR); Mijin LEE, Suwon-si (KR); Sung-Hyun JUNG, Suwon-si (KR); Ho Kuk JUNG, Suwon-si (KR)

(21) Appl. No.: 17/899,911

(22) Filed: Aug. 31, 2022

(30) Foreign Application Priority Data
Sep. 8, 2021 (KR) 10-2021-0119807
Aug. 26, 2022 (KR) 10-2022-0107851

(51) Int. Cl. H01L 51/00; C07D 403/14

(52) U.S. Cl. CPC H01L 51/0067; C07D 403/14; H01L 51/0072; H01L 51/5016

(57) ABSTRACT

Provided are a compound for an organic optoelectronic device represented by Chemical Formula 1, a composition for an organic optoelectronic device, an organic optoelectronic device including the same, and a display device. Details for Chemical Formula 1 are as defined in the specification.

### CROSS-REFERENCE TO RELATED APPLICATIONS

This application claims priority to and the benefit of Korean Patent Application No. 10-2021-0119807 filed in the Korean Intellectual Property Office on Sep. 8, 2021, and Korean Patent Application No. 10-2022-0107851, filed in the Korean Intellectual Property Office on Aug. 26, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy to optical energy, optical energy to electrical energy, or electrical energy and optical energy to each other.

An organic optoelectronic device may be classified as follows in accordance with its driving principles. One is a photoelectric device that generates electrical energy by separating excitons formed by light energy into electrons and holes, and transferring the electrons and holes to different electrodes, respectively. Another is a light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### SUMMARY

An embodiment is directed to a compound for an organic optoelectronic device, the compound being represented by Chemical Formula 1.

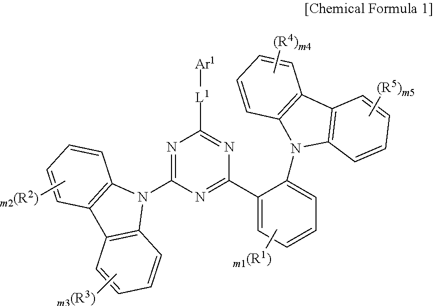

In Chemical Formula 1,

Ar¹ may be a substituted or unsubstituted C10 to C30 condensed aryl group,

L¹ may be a single bond or a substituted or unsubstituted C6 to C12 arylene group,

R¹ may be hydrogen, deuterium, or an unsubstituted C6 to C12 aryl group,

R² to R⁵ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

m1 to m5 may each independently be an integer of 1 to 4.

An embodiment is directed to a composition for an organic optoelectronic device including a first compound, and a second compound.

The first compound may be the same as described above, and the second compound may be a compound for an organic optoelectronic device represented by Chemical Formula 2; or a compound for an organic optoelectronic device represented by a combination of Chemical Formulas 3 and 4.

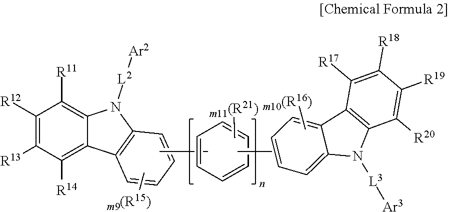

In Chemical Formula 2,

Ar² and Ar³ may each independently be a substituted or unsubstituted C6 to C20 aryl group or a substituted or unsubstituted C2 to C30 heterocyclic group,

L² and L³ may each independently be a single bond or a substituted or unsubstituted C6 to C20 arylene group,

R¹¹ to R²¹ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,

m9 and m10 may each independently be an integer of 1 to 3,

m11 may be an integer of 1 to 4, and

n may be an integer of 0 to 2.

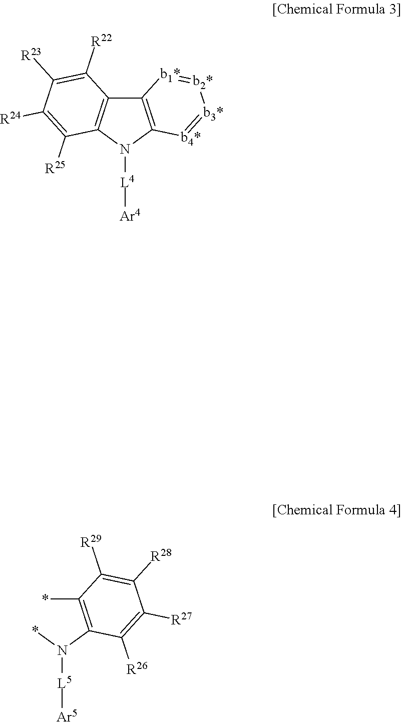

In Chemical Formulas 3 and 4,

Ar⁴ and Ar⁵ may each independently be a substituted or unsubstituted C6 to C20 aryl group or a substituted or unsubstituted C2 to C30 heterocyclic group,

adjacent two of b₁* to b₄* of Chemical Formula 3 may each be a carbon (C) linked to * of Chemical Formula 4,

of b₁* to b₄* in Chemical Formula 3, the other two not linked to * in Chemical Formula 4 may each independently be C-L^{a}-R^{a},

L^{a}, L⁴, and L⁵ may each independently be a single bond or a substituted or unsubstituted C6 to C20 arylene group, and

R^{a} and R²² to R²⁹ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

An embodiment is directed to an organic optoelectronic device that includes an anode and a cathode facing each other, and at least one organic layer between the anode and the cathode, wherein the at least one organic layer includes the compound for the organic optoelectronic device or the composition for the organic optoelectronic device.

An embodiment is directed to a display device including the organic optoelectronic device.

### BRIEF DESCRIPTION OF THE DRAWING

Features will become apparent to those of skill in the art by describing in detail exemplary embodiments with reference to the attached drawing in which:

The FIGURE is a cross-sectional view illustrating an organic light emitting diode according to an example embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art. In the drawing FIGURE, the dimensions of layers and regions may be exaggerated for clarity of illustration. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof.

As used herein, “condensed aryl group” refers to a form in which all carbon atoms of a hydrocarbon aromatic moiety have p-orbitals, and these p-orbitals form a conjugate, that is, an aryl group in the form of a fused ring polycyclic composed of a ring that shares adjacent pairs of carbon atoms.

More specifically, the substituted or unsubstituted C10 to C30 condensed aryl group may be a substituted or unsubstituted naphthyl group, a substituted or unsubstituted anthracenyl group, a substituted or unsubstituted phenanthrenyl group, a substituted or unsubstituted triphenylene group, a substituted or unsubstituted pyrenyl group, a substituted or unsubstituted chrysenyl group, a substituted or unsubstituted perylenyl group, a substituted or unsubstituted tetracene, a substituted or unsubstituted benzanthracene, or a combination thereof.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an example embodiment is described.

A compound for the organic optoelectronic device according to an embodiment is represented by Chemical Formula 1.

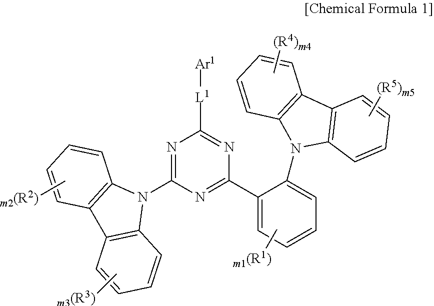

In an example embodiment of Chemical Formula 1,

Ar¹ is a substituted or unsubstituted C10 to C30 condensed aryl group,

L¹ is a single bond or a substituted or unsubstituted C6 to C12 arylene group,

R¹ is hydrogen, deuterium, or an unsubstituted C6 to C12 aryl group,

R² to R⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and

m1 to m5 are each independently an integer of 1 to 4.

The compound represented by Chemical Formula 1 may have a structure in which one carbazole group is directly linked to the triazine without a linking group in an N-direction centering on the triazine, and another carbazole group is linked to the triazine in an N-direction through ortho-phenylene.

One carbazole group is directly linked to the triazine without a linking group in the N-direction, that is, the 9th position. Without being bound by theory, it is believed that this provides a relatively deep LUMO energy level, which is advantageous for electron injection and movement.

In addition, the other carbazole group is linked to the triazine in the N-direction, that is, 9th position. Without being bound by theory, it is believed that this breaks 7r-bonding through the C—N bond, and the electron cloud between HOMO-LUMO may be clearly localized into a hole transport moiety and an electron transport moiety.

In particular, Without being bound by theory, it is believed that, since the HOMO-LUMO band gap is widened due to the ortho-phenylene, an efficiency improvement effect may be maximized, and a steric hindrance of molecules is increased, so that a deposition temperature is not relatively high, which is advantageous in the process.

Also, without being bound by theory, it is believed that an electron transport capability may be adjusted through the substitution of condensed aryl with triazine, and thus an appropriate charge balance in the light emitting layer may be adjusted to manufacture a low-driving, high-efficiency, and long life-span OLED device.

In an example embodiment of Chemical Formula 1, Ar¹ may be a substituted or unsubstituted C10 to C30 condensed aryl group, and the substituted or unsubstituted C10 to C30 condensed aryl group may be for example a substituted or unsubstituted naphthyl group, a substituted or unsubstituted anthracenyl group, a substituted or unsubstituted phenanthrenyl group, a substituted or unsubstituted triphenylene group, a substituted or unsubstituted pyrenyl group, a substituted or unsubstituted chrysenyl group, a substituted or unsubstituted perylenyl group, a substituted or unsubstituted tetracene, a substituted or unsubstituted benzanthracene, or a combination thereof.

In an example embodiment, the substituted or unsubstituted C10 to C30 condensed aryl group may be selected from the substituents of Group I.

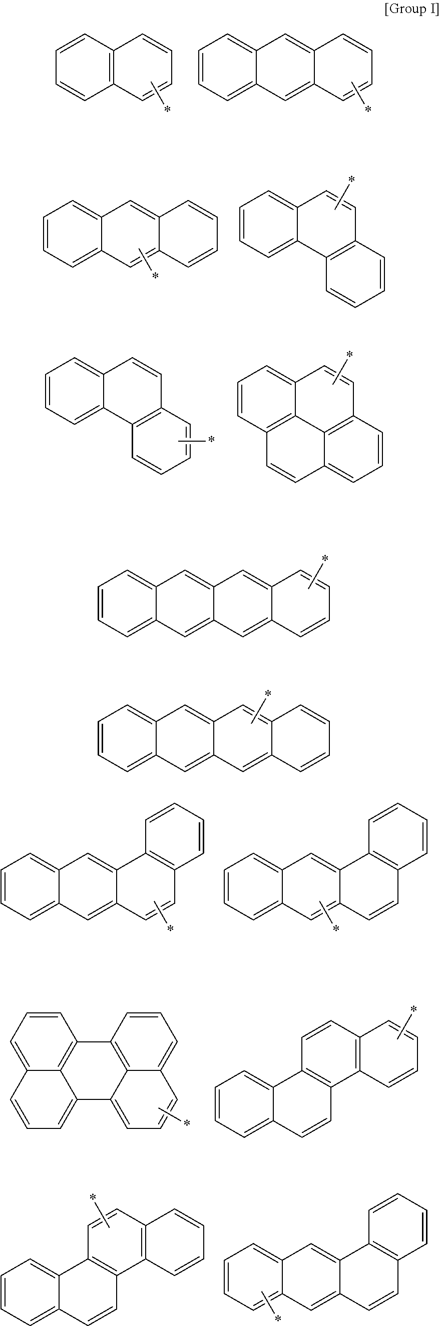

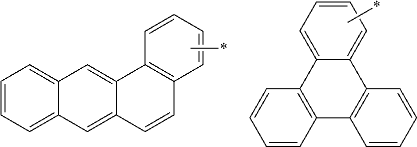

In Group I,

* is a linking point, and

substituents in Group I may be unsubstituted or substituted with additional substituents. The additional substituent may be deuterium, a halogen group, a hydroxyl group, an amino group, a C1 to C10 alkyl group, a C6 to C20 aryl group, a C2 to C20 heteroaryl group, or a cyano group. In one example, the additional substituent may be deuterium, a halogen group, a hydroxyl group, a C1 to C10 alkyl group, a C6 to C12 aryl group, or a cyano group. In a specific example, the additional substituent may be deuterium, a C1 to C5 alkyl group, a cyano group, a phenyl group, a biphenyl group, a terphenyl group, or a naphthyl group.

In an example embodiment of Chemical Formula 1, Ar¹ may be a substituted or unsubstituted naphthyl group, a substituted or unsubstituted phenanthrenyl group, or a substituted or unsubstituted triphenylene group.

In an example embodiment of Chemical Formula 1, L¹ may be a single bond or a substituted or unsubstituted phenylene group. In an example embodiment, L¹ may be a single bond.

In an example embodiment of Chemical Formula 1, R¹ and R² may each independently be hydrogen, deuterium, or a substituted or unsubstituted phenyl group. In an example embodiment, R¹ and R² may each independently be hydrogen or deuterium.

In an example embodiment of Chemical Formula 1, R³ to R⁵ may each independently be hydrogen, deuterium, a substituted or unsubstituted C1 to C10 alkyl group, or a substituted or unsubstituted C6 to C12 aryl group. In an example embodiment, R³ to R⁵ may each independently be hydrogen, deuterium, or a substituted or unsubstituted phenyl group.

Examples of the compound for the organic optoelectronic device represented by Chemical Formula 1 may include the compounds of Group 1.

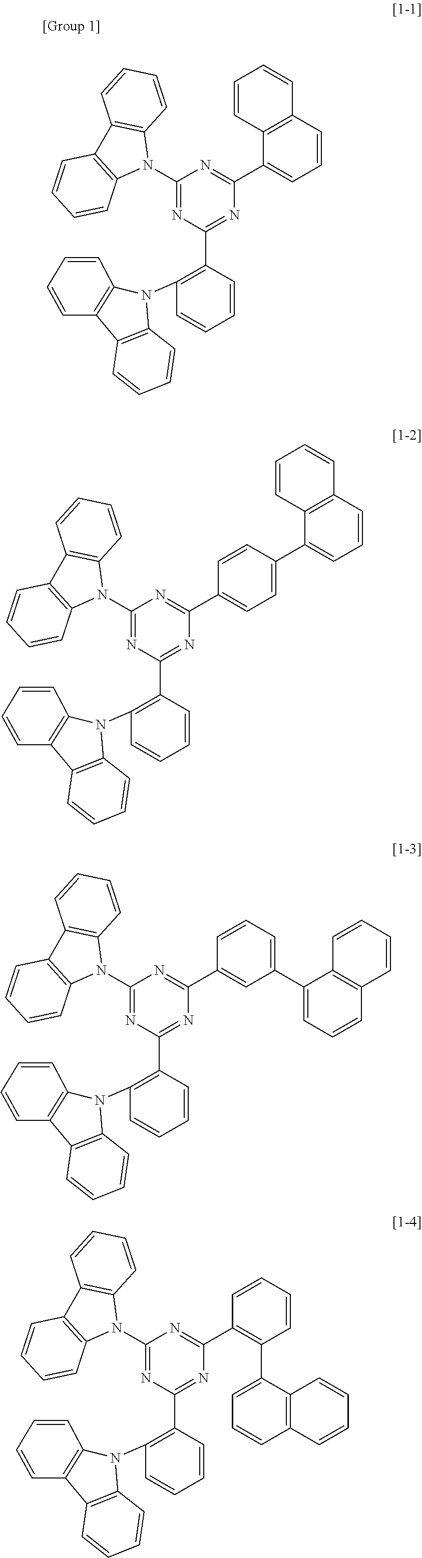

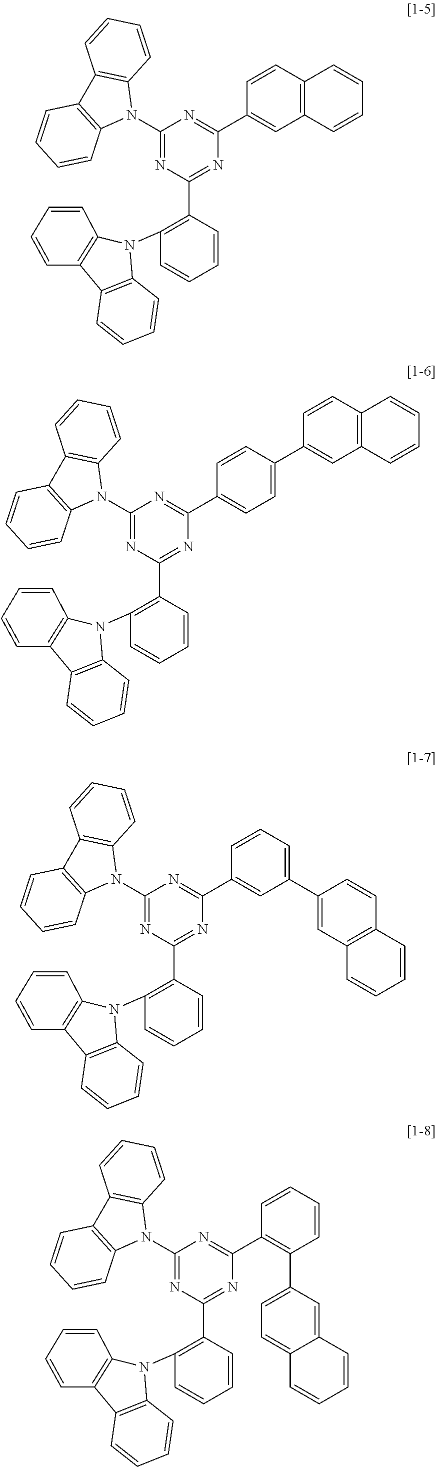

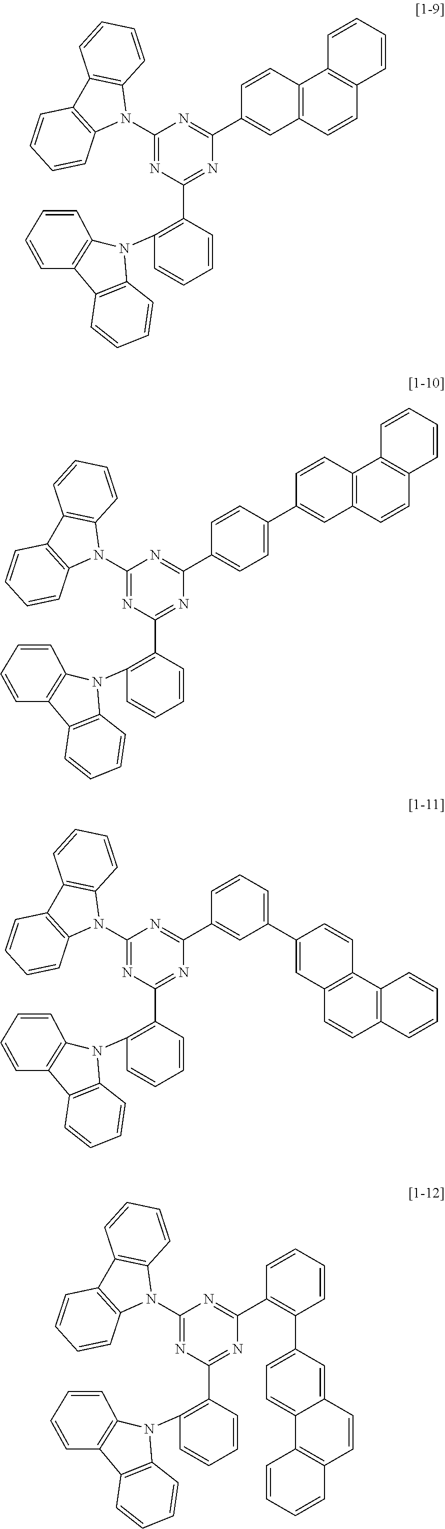 [SMILES: c1ccc(-n2c3ccccc3c3ccccc32)c(-c2nc(-c3ccc4c(ccc5ccccc54)c3)nc(-n3c4ccccc4c4ccccc43)n2)c1 ; c1ccc(-n2c3ccccc3c3ccccc32)c(-c2nc(-c3ccc(-c4ccc5c(ccc6ccccc65)c4)cc3)nc(-n3c4ccccc4c4ccccc43)n2)c1 ; c1cc(-c2ccc3c(ccc4ccccc43)c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(ccc4ccccc43)c2)c1]

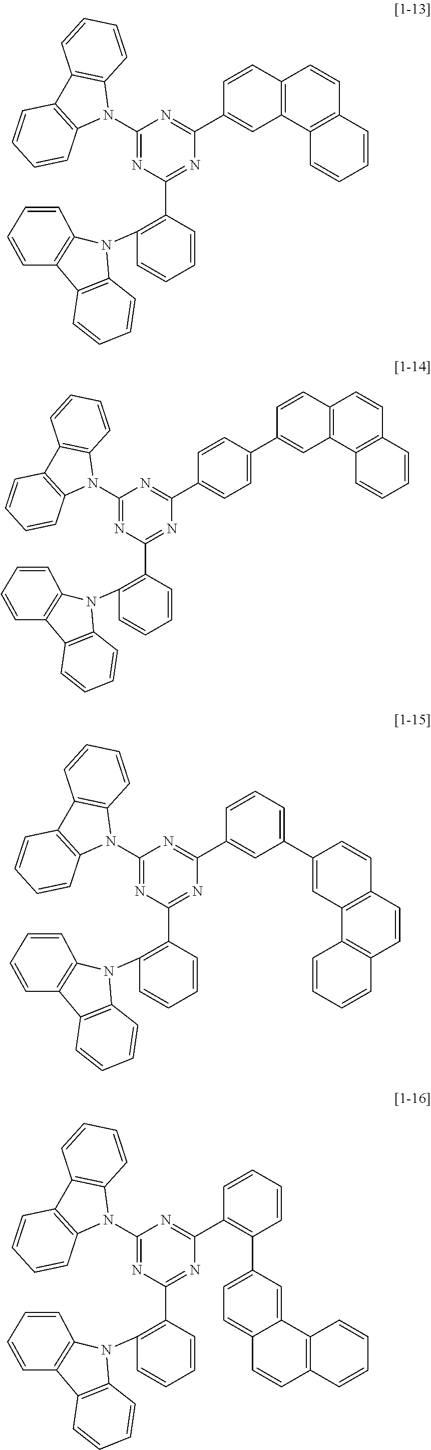 [SMILES: c1ccc(-n2c3ccccc3c3ccccc32)c(-c2nc(-c3ccc4ccc5ccccc5c4c3)nc(-n3c4ccccc4c4ccccc43)n2)c1 ; c1ccc(-n2c3ccccc3c3ccccc32)c(-c2nc(-c3ccc(-c4ccc5ccc6ccccc6c5c4)cc3)nc(-n3c4ccccc4c4ccccc43)n2)c1 ; c1cc(-c2ccc3ccc4ccccc4c3c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3ccc4ccccc4c3c2)c1]

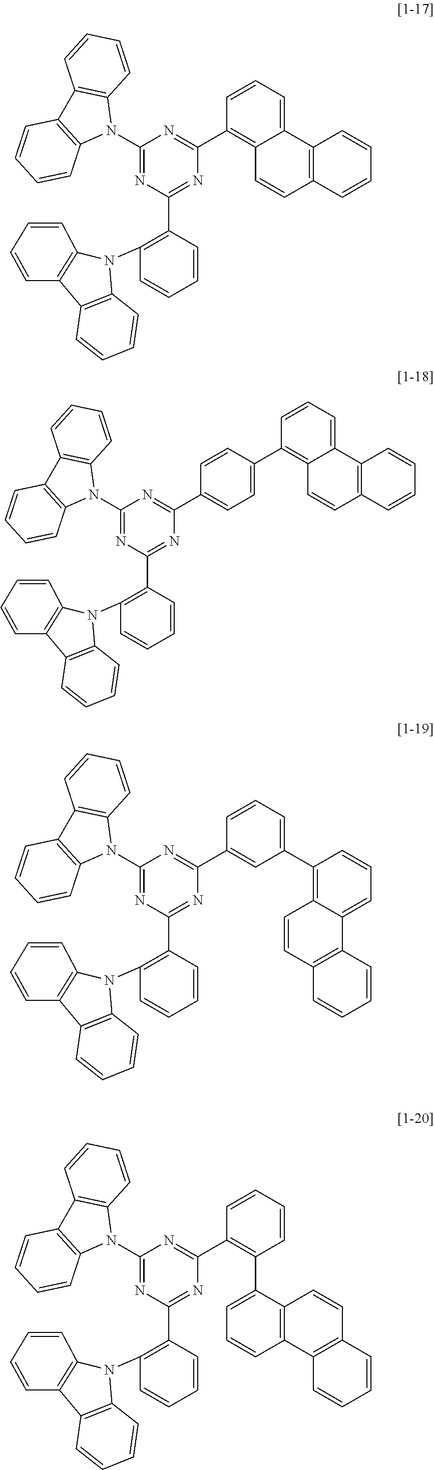 [SMILES: c1ccc(-n2c3ccccc3c3ccccc32)c(-c2nc(-c3cccc4c3ccc3ccccc34)nc(-n3c4ccccc4c4ccccc43)n2)c1 ; c1ccc(-n2c3ccccc3c3ccccc32)c(-c2nc(-c3ccc(-c4cccc5c4ccc4ccccc45)cc3)nc(-n3c4ccccc4c4ccccc43)n2)c1 ; c1cc(-c2nc(-c3ccccc3-n3c4ccccc4c4ccccc43)nc(-n3c4ccccc4c4ccccc43)n2)cc(-c2cccc3c2ccc2ccccc23)c1 ; c1ccc(-c2cccc3c2ccc2ccccc23)c(-c2nc(-c3ccccc3-n3c4ccccc4c4ccccc43)nc(-n3c4ccccc4c4ccccc43)n2)c1]

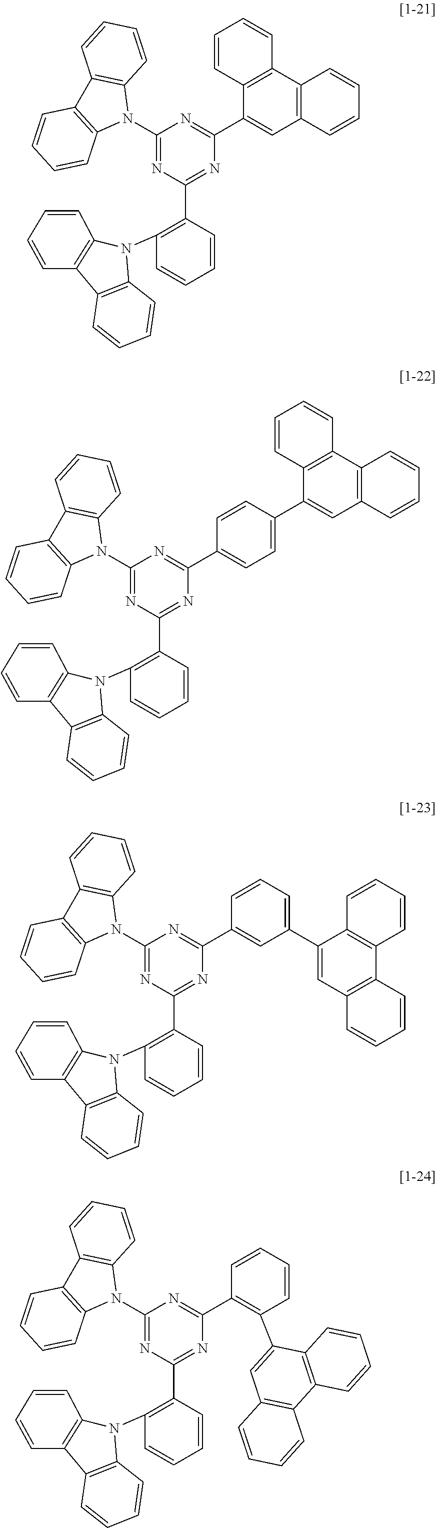

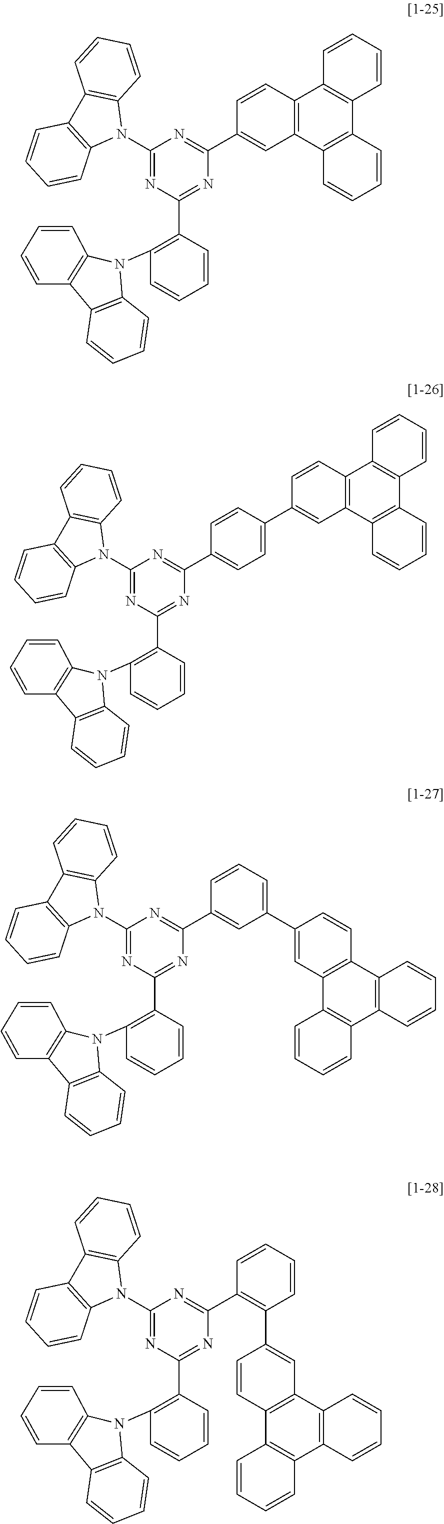 [SMILES: c1ccc(-n2c3ccccc3c3ccccc32)c(-c2nc(-c3ccc4c5ccccc5c5ccccc5c4c3)nc(-n3c4ccccc4c4ccccc43)n2)c1 ; c1ccc(-n2c3ccccc3c3ccccc32)c(-c2nc(-c3ccc(-c4ccc5c6ccccc6c6ccccc6c5c4)cc3)nc(-n3c4ccccc4c4ccccc43)n2)c1 ; c1cc(-c2ccc3c4ccccc4c4ccccc4c3c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c4ccccc4c4ccccc4c3c2)c1]

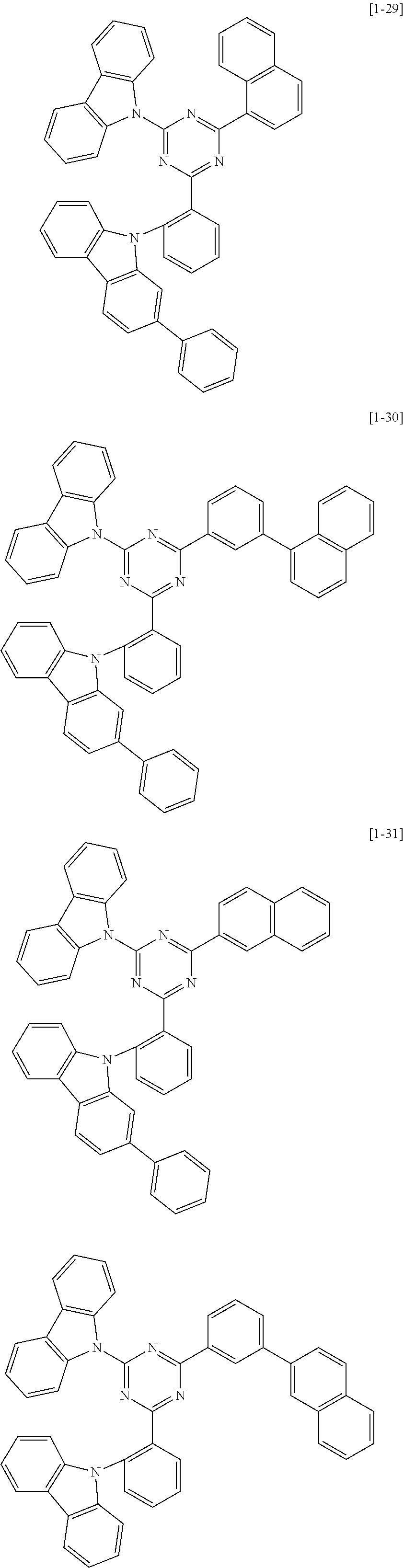 [SMILES: c1ccc(-c2ccc3c4ccccc4n(-c4ccccc4-c4nc(-c5cccc6ccccc56)nc(-n5c6ccccc6c6ccccc65)n4)c3c2)cc1 ; c1ccc(-c2ccc3c4ccccc4n(-c4ccccc4-c4nc(-c5cccc(-c6cccc7ccccc67)c5)nc(-n5c6ccccc6c6ccccc65)n4)c3c2)cc1 ; c1ccc(-c2ccc3c4ccccc4n(-c4ccccc4-c4nc(-c5ccc6ccccc6c5)nc(-n5c6ccccc6c6ccccc65)n4)c3c2)cc1 ; c1cc(-c2ccc3ccccc3c2)cc(-c2nc(-c3ccccc3-n3c4ccccc4c4ccccc43)nc(-n3c4ccccc4c4ccccc43)n2)c1]

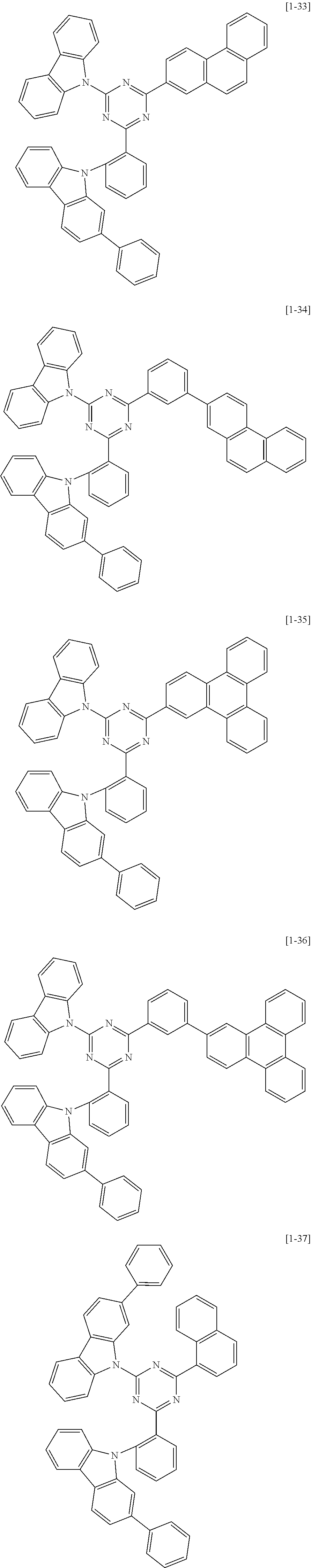

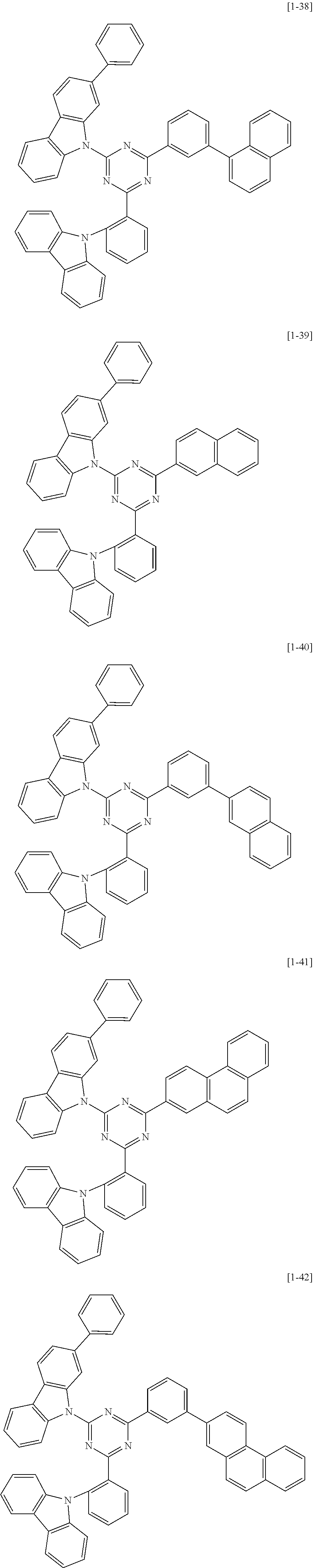

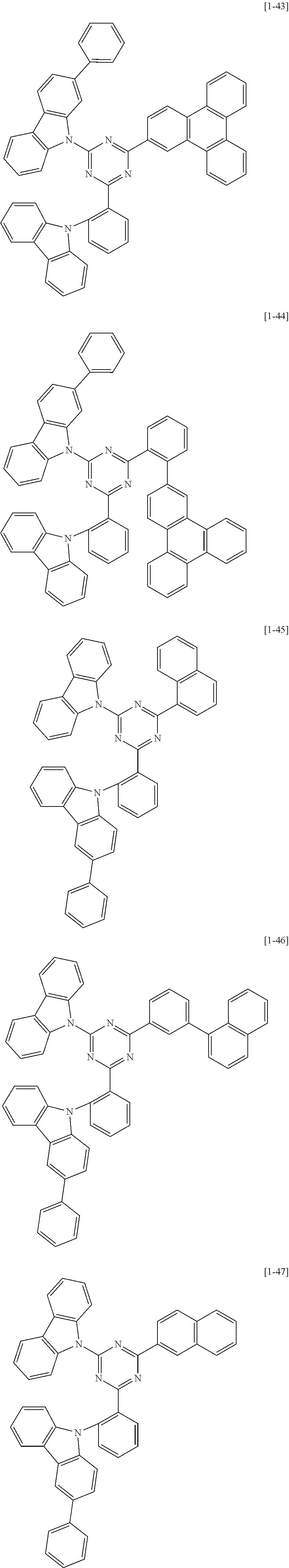

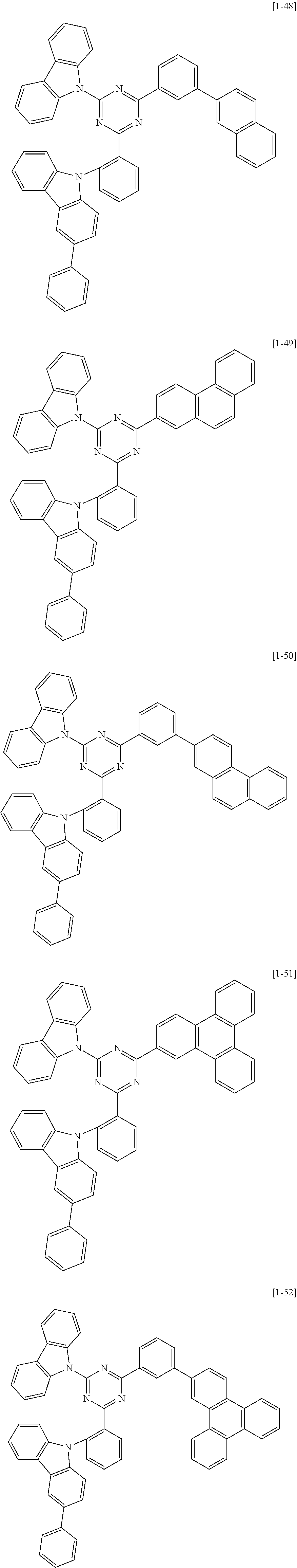

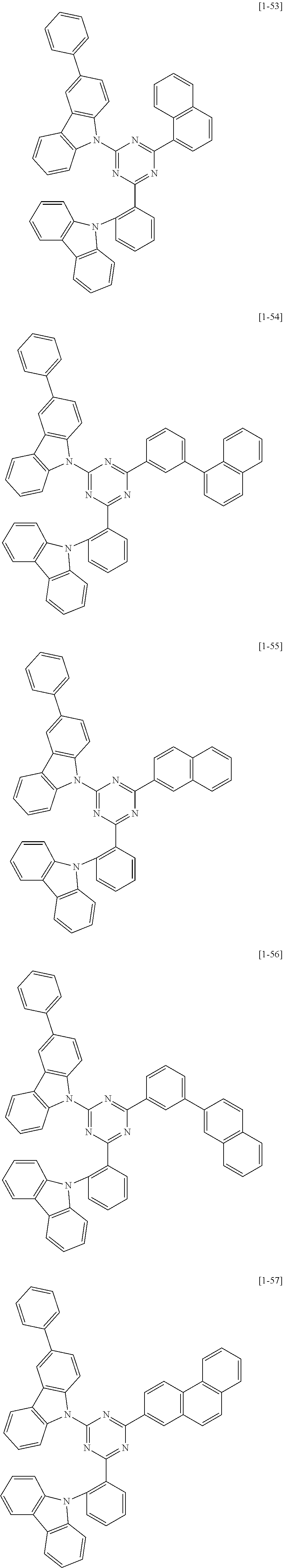 [SMILES: c1ccc(-c2ccc3c(c2)c2ccccc2n3-c2nc(-c3ccccc3-n3c4ccccc4c4ccccc43)nc(-c3cccc4ccccc34)n2)cc1 ; c1ccc(-c2ccc3c(c2)c2ccccc2n3-c2nc(-c3cccc(-c4cccc5ccccc45)c3)nc(-c3ccccc3-n3c4ccccc4c4ccccc43)n2)cc1 ; c1ccc(-c2ccc3c(c2)c2ccccc2n3-c2nc(-c3ccc4ccccc4c3)nc(-c3ccccc3-n3c4ccccc4c4ccccc43)n2)cc1 ; c1ccc(-c2ccc3c(c2)c2ccccc2n3-c2nc(-c3cccc(-c4ccc5ccccc5c4)c3)nc(-c3ccccc3-n3c4ccccc4c4ccccc43)n2)cc1 ; c1ccc(-c2ccc3c(c2)c2ccccc2n3-c2nc(-c3ccc4c(ccc5ccccc54)c3)nc(-c3ccccc3-n3c4ccccc4c4ccccc43)n2)cc1]

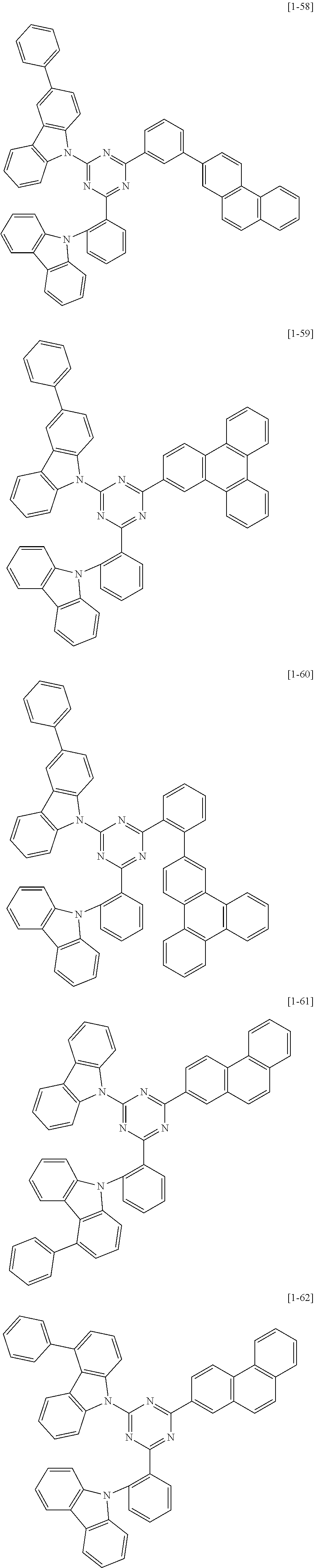

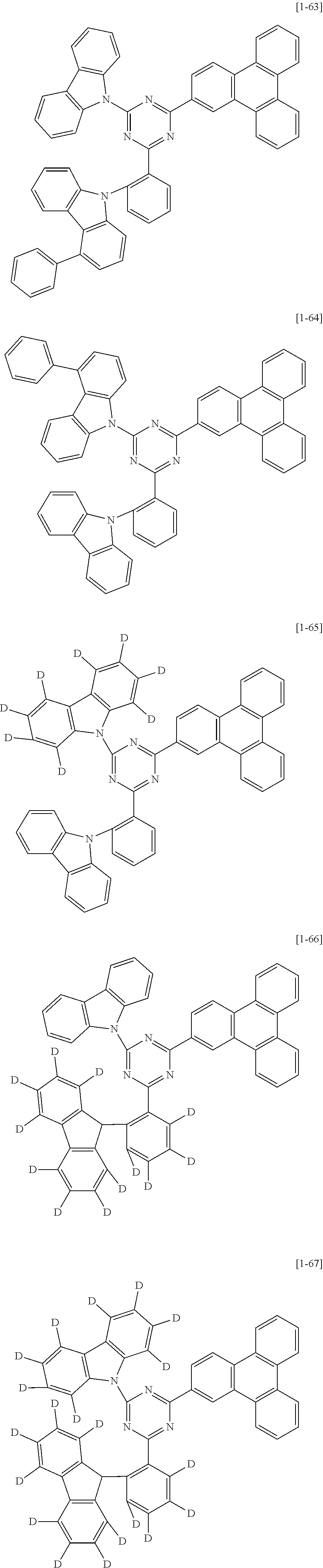

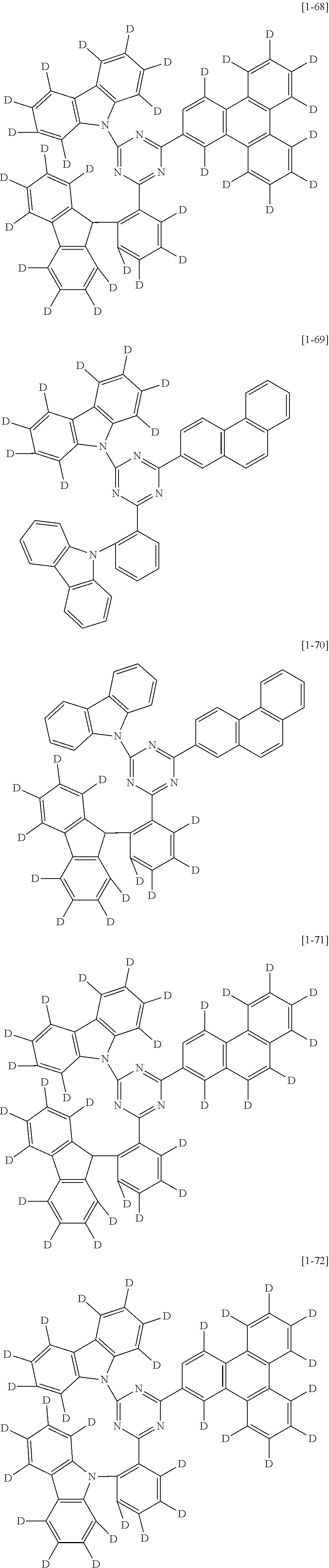

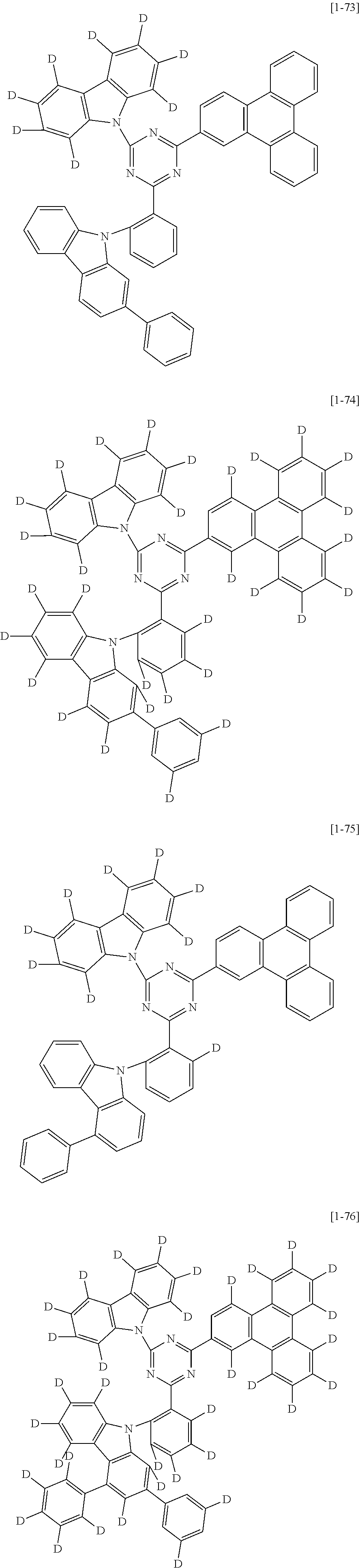

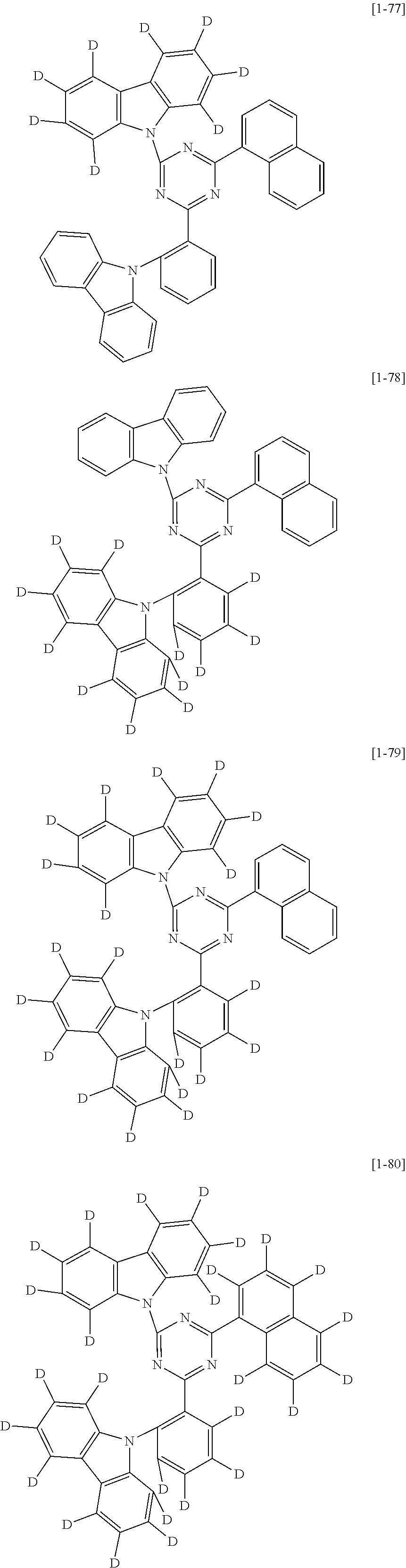

According to another example embodiment, a composition for an organic optoelectronic device according includes a first compound and a second compound. The first compound may be the aforementioned compound for the organic optoelectronic device represented by Chemical Formula 1. The second compound may be a compound for an organic optoelectronic device represented by Chemical Formula 2; or a compound for an organic optoelectronic device represented by a combination of Chemical Formulas 3 and 4.

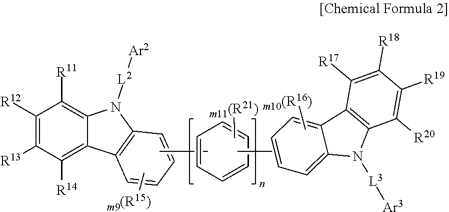

In an example embodiment of Chemical Formula 2,

Ar² and Ar³ are each independently a substituted or unsubstituted C6 to C20 aryl group or a substituted or unsubstituted C2 to C30 heterocyclic group,

L² and L³ are each independently a single bond or a substituted or unsubstituted C6 to C20 arylene group,

R¹¹ to R²¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,

m9 and m10 are each independently an integer of 1 to 3,

m11 is an integer of 1 to 4, and

n is an integer of 0 to 2.

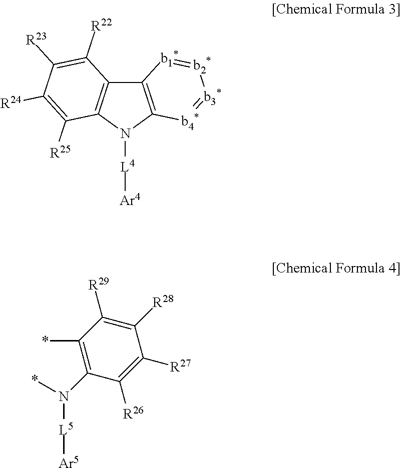

In an example embodiment of Chemical Formulas 3 and 4,

Ar⁴ and Ar⁵ are each independently a substituted or unsubstituted C6 to C20 aryl group or a substituted or unsubstituted C2 to C30 heterocyclic group,

adjacent two of b₁* to b₄* of Chemical Formula 3 are each a carbon (C) linked to * of Chemical Formula 4,

of b₁* to b₄* in Chemical Formula 3, the other two not linked to * in Chemical Formula 4 are each independently C-L^{a}-R^{a},

L^{a}, L⁴, and L⁵ are each independently a single bond or a substituted or unsubstituted C6 to C20 arylene group, and

R^{a} and R²² to R²⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

The second compound may be used in the light emitting layer together with the first compound to improve luminous efficiency and life-span characteristics by increasing charge mobility and increasing stability.

In an example embodiment of Chemical Formula 2,

Ar² and Ar³ may each independently be a substituted or unsubstituted C6 to C20 aryl group or a substituted or unsubstituted C2 to C30 heterocyclic group, and

at least one of Ar² and Ar³ may be a C6 to C20 aryl group substituted with deuterium or a C2 to C30 heterocyclic group substituted with deuterium.

In an example embodiment of Chemical Formula 2,

R¹¹ to R²¹ may each independently be hydrogen, deuterium, a cyano group, a halogen group, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

at least one of R¹¹ to R²¹ may be deuterium or a C1 to C30 alkyl group substituted with deuterium, a C6 to C30 aryl group substituted with deuterium, or a C2 to C30 heterocyclic group substituted with deuterium.

In an example embodiment of Chemical Formula 2,

at least one of Ar² and Ar³ may be a C6 to C20 aryl group substituted with deuterium, or a C2 to C30 heterocyclic group substituted with deuterium, and

at least one of R¹¹ to R²¹ may be deuterium or a C1 to C30 alkyl group substituted with deuterium, a C6 to C30 aryl group substituted with deuterium, or a C2 to C30 heterocyclic group substituted with deuterium.

In an example embodiment of Chemical Formulas 3 and 4,

Ar⁴ and Ar⁵ may each independently be a substituted or unsubstituted C6 to C20 aryl group or a substituted or unsubstituted C2 to C30 heterocyclic group, and

at least one of Ar⁴ and Ar⁵ may be a C6 to C20 aryl group substituted with deuterium, or a C2 to C30 heterocyclic group substituted with deuterium.

In an example embodiment of Chemical Formulas 3 and 4,

R^{a} and R²² to R²⁹ may each independently be hydrogen, deuterium, a cyano group, a halogen group, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

at least one of R^{a} and R²² to R²⁹ may be deuterium, a C1 to C30 alkyl group substituted with deuterium, a C6 to C30 aryl group substituted with deuterium, or a C2 to C30 heterocyclic group substituted with deuterium.

In an example embodiment of Chemical Formulas 3 and 4,

at least one of Ar⁴ and Ar⁵ may be a C6 to C20 aryl group substituted with deuterium, or a C2 to C30 heterocyclic group substituted with deuterium, and

at least one of R^{a} and R²² to R²⁹ may be deuterium, a C1 to C30 alkyl group substituted with deuterium, a C6 to C30 aryl group substituted with deuterium, or a C2 to C30 heterocyclic group substituted with deuterium.

In an example embodiment of Chemical Formula 2,

Ar² and Ar³ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,

L² and L³ may each independently be a single bond, a substituted or unsubstituted phenylene group, or a substituted or unsubstituted biphenylene group,

R¹¹ to R²¹ may each independently be hydrogen, deuterium, or a substituted or unsubstituted C6 to C12 aryl group, and

n may be 0 or 1.

In an example embodiment of Chemical Formula 2,

Ar² and Ar³ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group, and

at least one of Ar² and Ar³ may be a phenyl group substituted with deuterium, a biphenyl group substituted with deuterium, a terphenyl group substituted with deuterium, a naphthyl group substituted with deuterium, an anthracenyl group substituted with deuterium, a triphenylenyl group substituted with deuterium, a carbazolyl group substituted with deuterium, a dibenzothiophenyl group substituted with deuterium, a dibenzofuranyl group substituted with deuterium, or a fluorenyl group substituted with deuterium.

In an example embodiment of Chemical Formula 2,

R¹¹ to R²¹ may each independently be hydrogen, deuterium, or a substituted or unsubstituted C6 to C12 aryl group, and

at least one of R¹¹ to R²¹ may be deuterium or a C6 to C12 aryl group substituted with deuterium.

In an example embodiment of Chemical Formula 2,

at least one of Ar² and Ar³ may be a phenyl group substituted with deuterium, a biphenyl group substituted with deuterium, a terphenyl group substituted with deuterium, a naphthyl group substituted with deuterium, an anthracenyl group substituted with deuterium, a triphenylenyl group substituted with deuterium, a carbazolyl group substituted with deuterium, a dibenzothiophenyl group substituted with deuterium, a dibenzofuranyl group substituted with deuterium, or a fluorenyl group substituted with deuterium, and

at least one of R¹¹ to R²¹ may be deuterium or a C6 to C12 aryl group substituted with deuterium.

In an example embodiment of Chemical Formula 2, “substituted” means that at least one hydrogen is replaced by deuterium, a C1 to C4 alkyl group, a C6 to C18 aryl group, or a C2 to C30 heteroaryl group.

In an example embodiment, Chemical Formula 2 may be represented by one of Chemical Formulas 2-1 to 2-15.

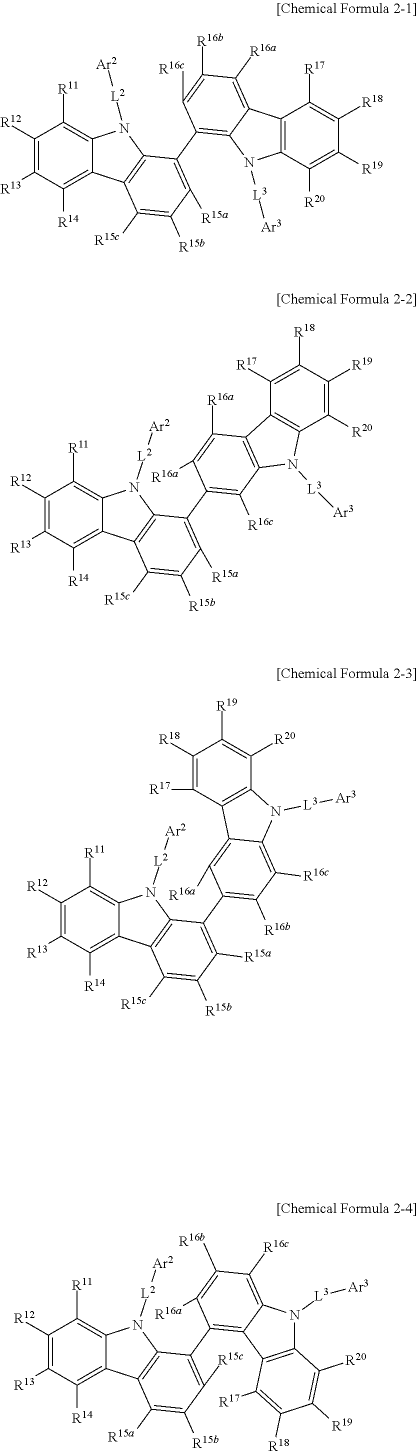

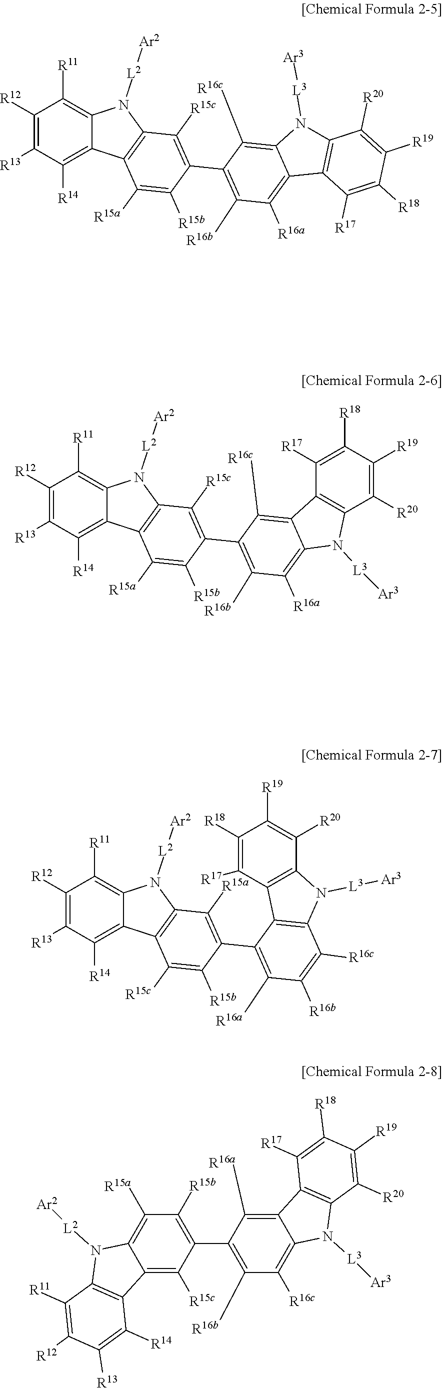

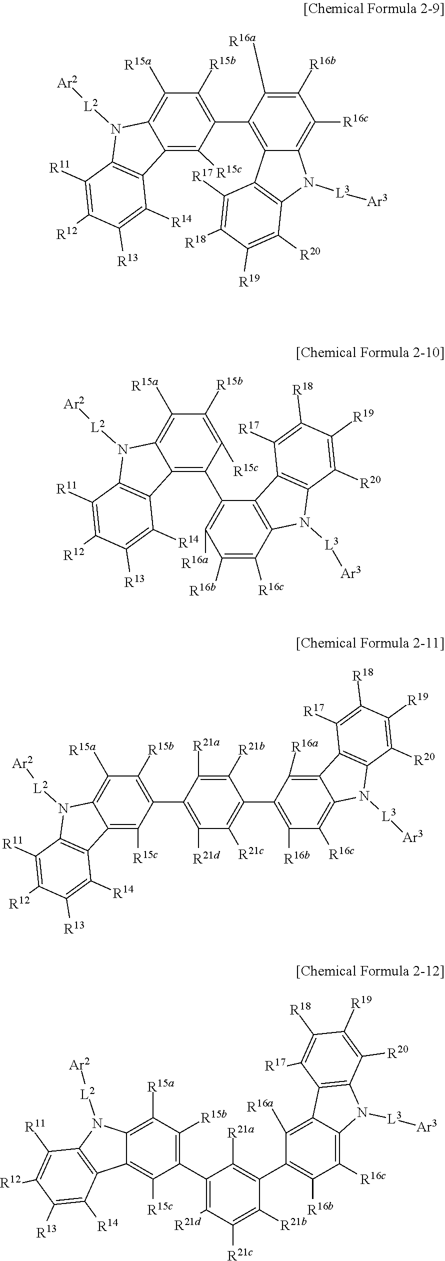

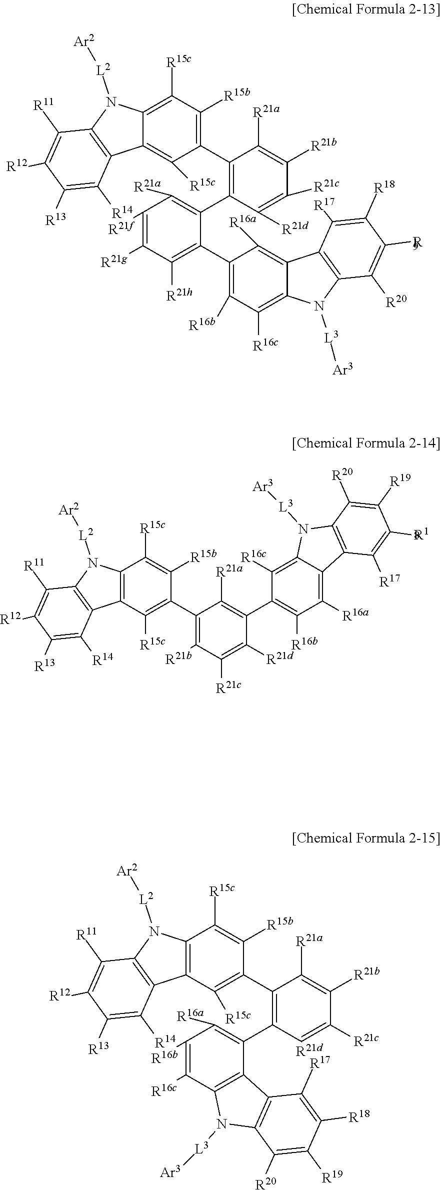

In Chemical Formula 2-1 to Chemical Formula 2-15,

R¹¹ to R¹⁴, R^{15a}, R^{15b}, R^{15c}, R^{16a}, R^{16b}, R^{16c}, R¹⁷ to R²⁰, R^{21a}, R^{21b}, R^{21c}, R^{21a}, R^{21e}, R^{21f}, R^{21g}, and R^{21h} may each independently be hydrogen, deuterium, or a substituted or unsubstituted C6 to C12 aryl group, and

*-L²-Ar² and *-L³-Ar³ may each independently be one of substituents of Group II.

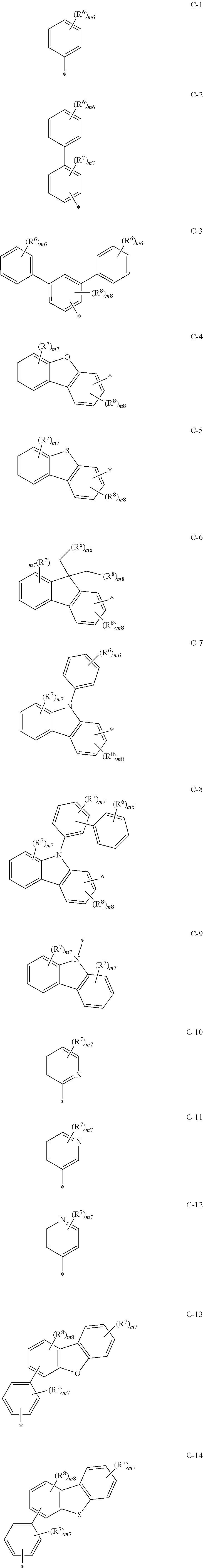

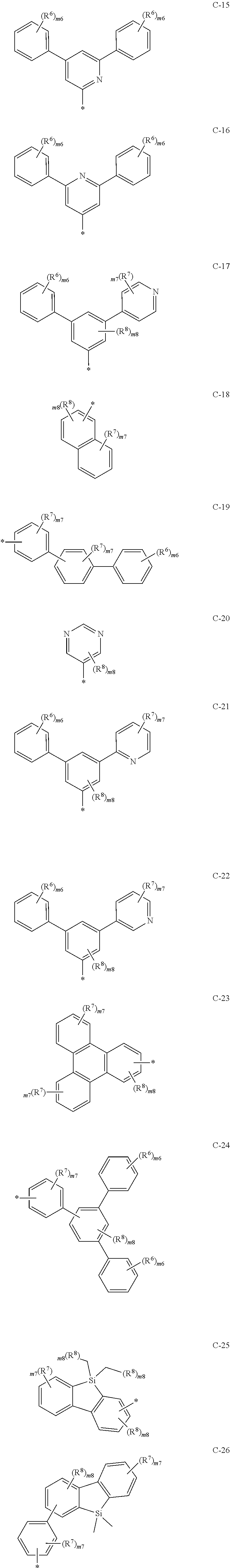

In Group II,

R⁶ to R⁸ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,

m6 is an integer of 1 to 5,

m7 is an integer of 1 to 4,

m8 is an integer of 1 to 3, and

* is a linking point.

In an example embodiment, Chemical Formula 2 may be represented by Chemical Formula 2-8.

In addition, *-L²-Ar² and *-L³-Ar³ of Chemical Formula 2-8 may each independently be selected from Group II, for example, one of C-1, C-2, C-3, C-4, C-7, C-8, and C-9.

In an example embodiment, the second compound represented by the combination of Chemical Formulas 3 and 4 may be represented by any one of Chemical Formula 3A, Chemical Formula 3B, Chemical Formula 3C, Chemical Formula 3D, or Chemical Formula 3E.

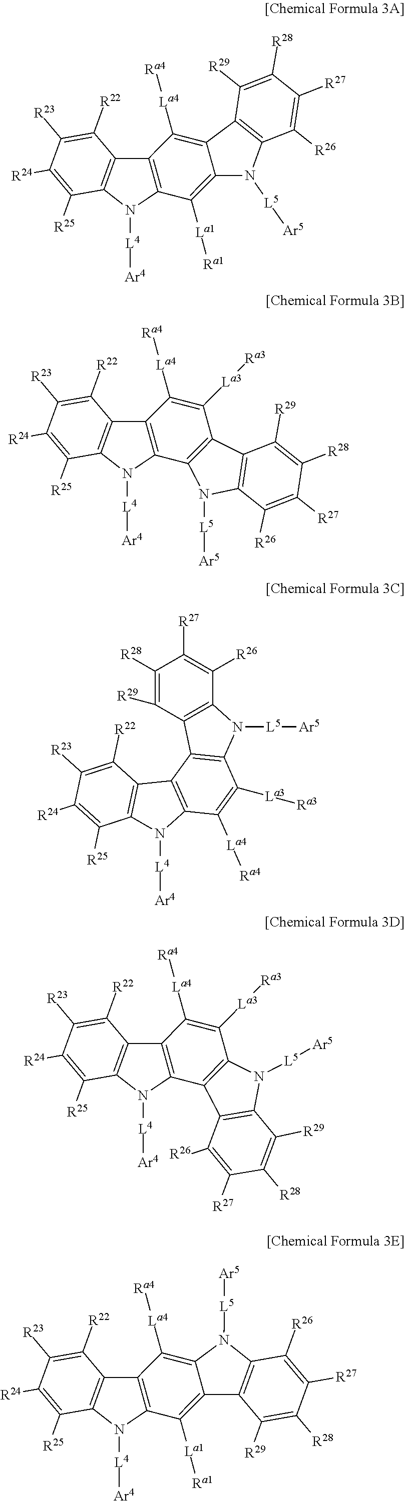

In Chemical Formula 3A to Chemical Formula 3E,

Ar⁴, Ar⁵, L⁴, L⁵, and R²² to R²⁹ are the same as described above,

L^{a1} to L^{a4} are the same as the definitions of L⁴ and L⁵, and

R^{a1} to R^{a4} are the same as the definitions of R²² to R²⁹.

In an example embodiment, in Chemical Formulas 3 and 4,

Ar⁴ and Ar⁵ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and

R^{a1} to R^{a4} and R²² to R²⁹ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an example embodiment, in Chemical Formulas 3 and 4, *-L⁴-Ar⁴ and *-L⁵-Ar⁵ may each independently be selected from the substituents listed in Group II.

In an example embodiment, R^{a1} to R^{a4} and R²² to R²⁹ may each independently be hydrogen, deuterium, a cyano group, a substituted or unsubstituted phenyl group, a substituted or unsubstituted pyridinyl group, a substituted or unsubstituted carbazolyl group, a substituted or unsubstituted dibenzofuranyl group, or a substituted or unsubstituted dibenzothiophenyl group. For example, R^{a1} to R^{a4} and R²² to R²⁹ may each independently be hydrogen, deuterium, a cyano group, or a substituted or unsubstituted phenyl group, and in a specific embodiment, R^{a1} to R^{a4}, and R²² to R²⁹ may each independently be hydrogen, deuterium, or a phenyl group.

In an example embodiment, in Chemical Formulas 3 and 4,

Ar⁴ and Ar⁵ may each independently be a substituted or unsubstituted phenyl group, a substituted or unsubstituted biphenyl group, a substituted or unsubstituted pyridinyl group, a substituted or unsubstituted carbazolyl group, or a substituted or unsubstituted dibenzofuranyl group, or a substituted or unsubstituted dibenzothiophenyl group, and

at least one of Ar⁴ and Ar⁵ may be a phenyl group substituted with deuterium, a biphenyl group substituted with deuterium, a pyridinyl group substituted with deuterium, a carbazolyl group substituted with deuterium, a dibenzofuranyl group substituted with deuterium, a dibenzofuranyl group substituted with deuterium, or a dibenzothiophenyl group substituted with deuterium.

In an example embodiment, in Chemical Formulas 3 and 4,

R^{a1} to R^{a4} and R²² to R²⁹ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and

at least one of R^{a1} to R^{a4} and R²² to R²⁹ may be deuterium, a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an example embodiment, in Chemical Formulas 3 and 4,

at least one of Ar⁴ and Ar⁵ may be a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium, and

at least one of R^{a1} to R^{a4} and R²² to R²⁹ may be deuterium, a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an example embodiment, the second compound may be represented by Chemical Formula 2-8, wherein in Chemical Formula 2-8, Ar² and Ar³ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L² and L³ may each independently be a single bond, or a substituted or unsubstituted C6 to C20 arylene group, and R¹¹ to R¹⁴, R^{15a}, R^{15b}, R^{15c}, R^{16a}, R^{16b}, R^{16c}, and R¹⁷ to R²⁰ may each independently be hydrogen, deuterium, a cyano group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an example embodiment of Chemical Formula 2-8, *-L²-Ar² and *-L³-Ar³ may each independently be selected from substituents of Group II.

In an example embodiment, the second compound may be represented by Chemical Formula 3C, wherein in Chemical Formula 3C, L^{a1} to L^{a4} may be a single bond, L⁴ and L⁵ may each independently be a single bond or a substituted or unsubstituted is a C6 to C12 arylene group, R²² to R²⁹, R^{a1} to R^{a4} may each be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and Ar⁴ and Ar⁵ may each independently be a substituted or unsubstituted phenyl group, a substituted or unsubstituted biphenyl group, a substituted or unsubstituted terphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an example embodiment of Chemical Formula 3C, *-L⁴-Ar⁴ and *-L⁵-Ar⁵ may each independently be selected from substituents of Group II.

In an example embodiment, the second compound may be one selected from the compounds of Group 2.

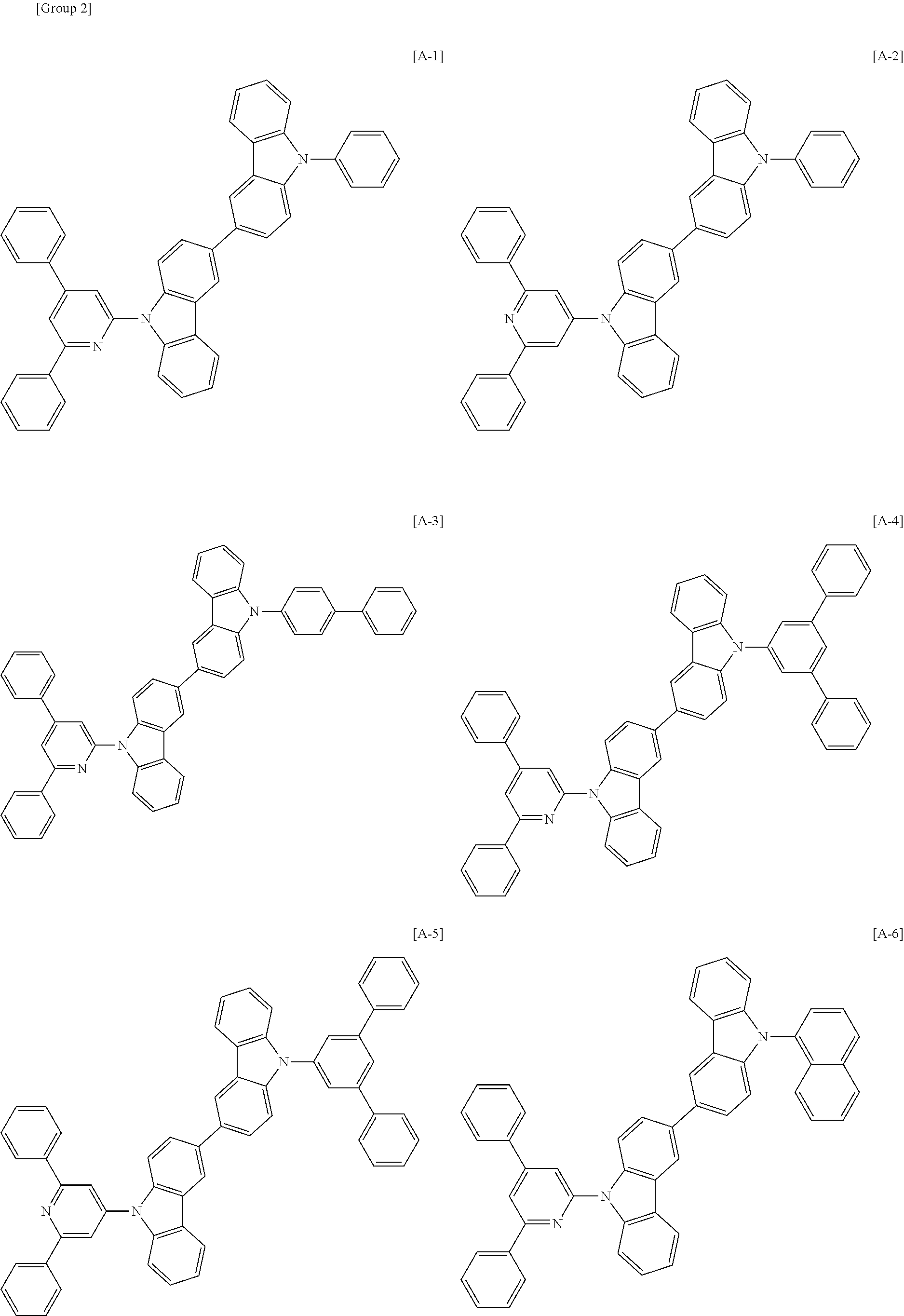

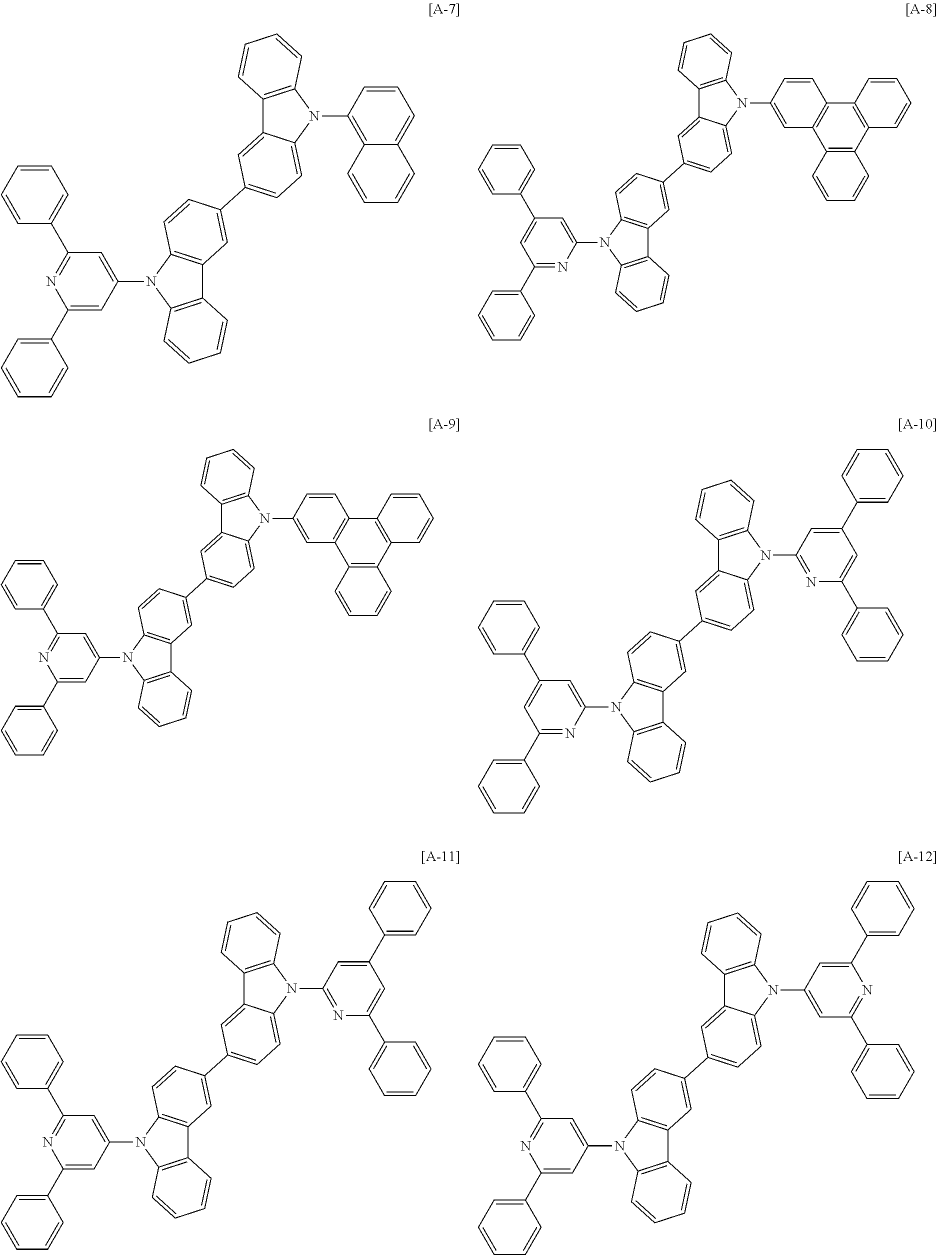

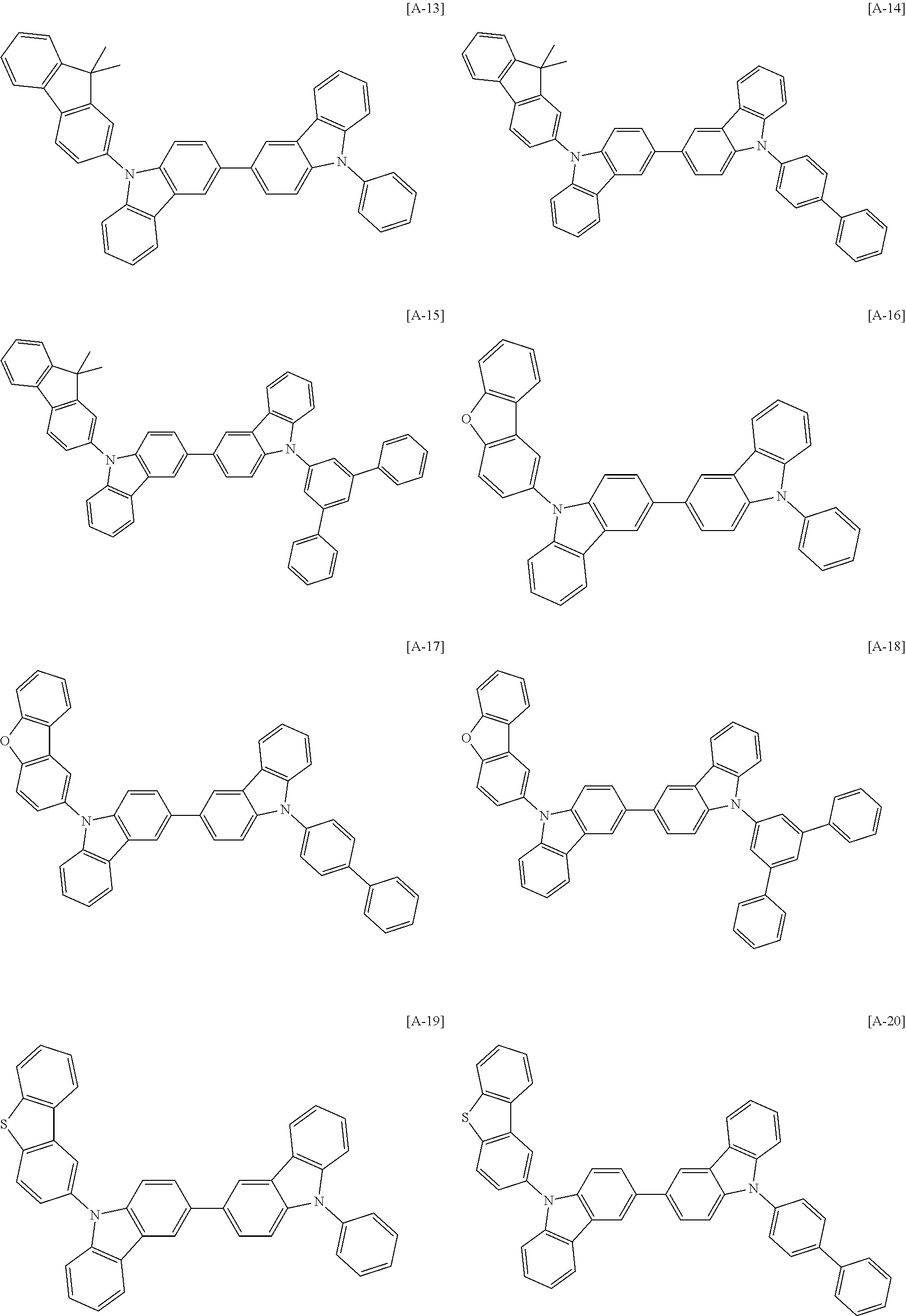

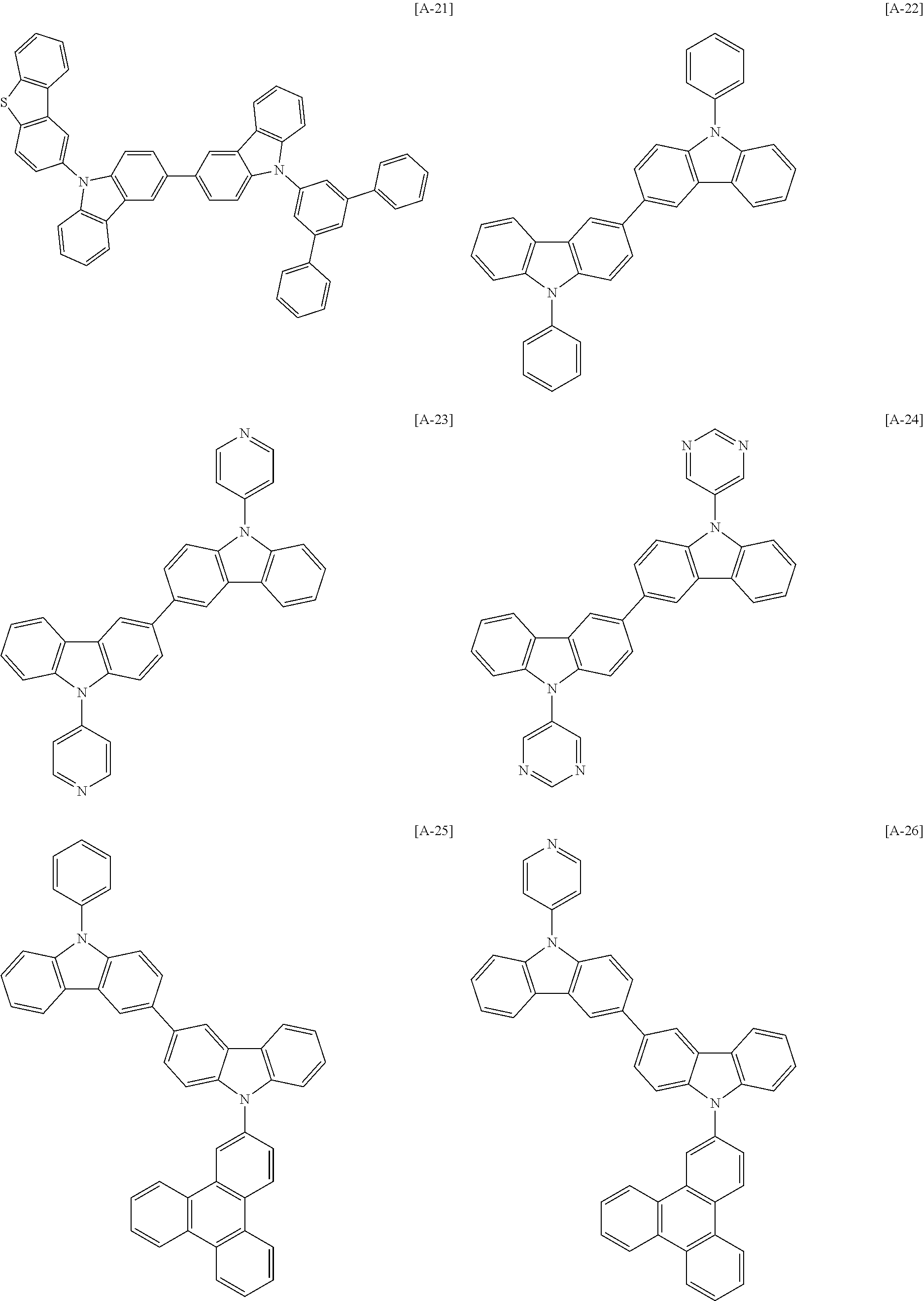 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

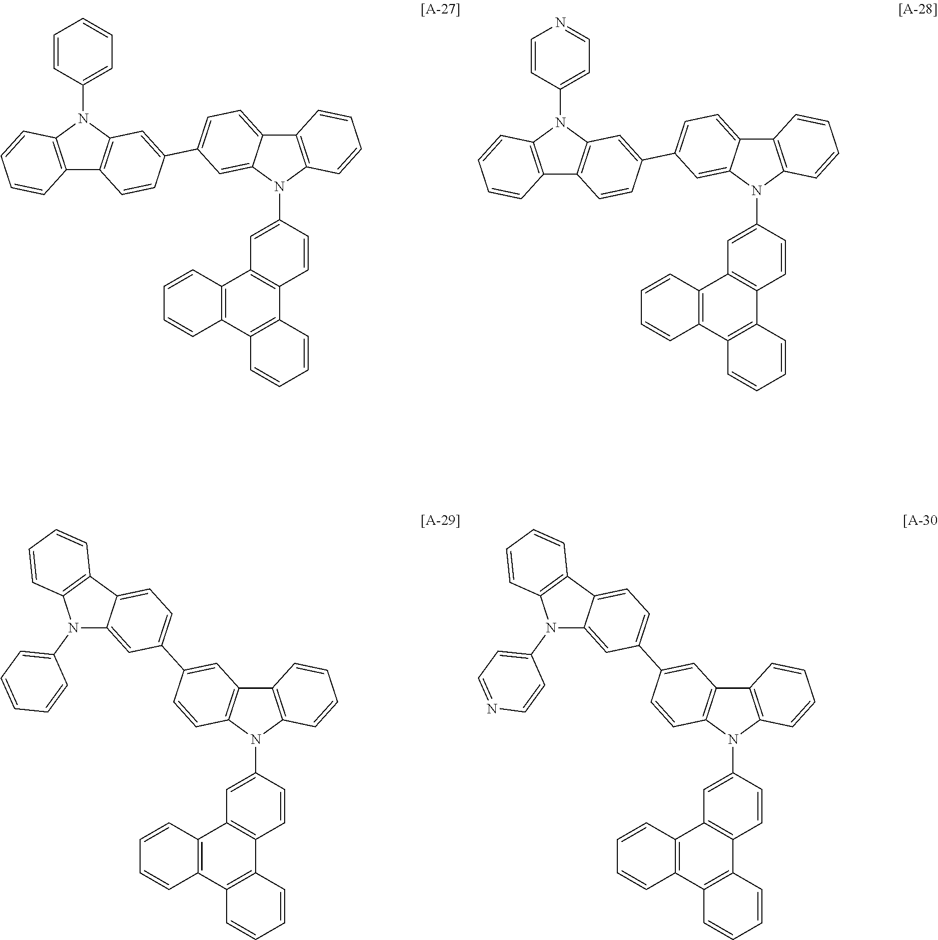 [SMILES: c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21 ; c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

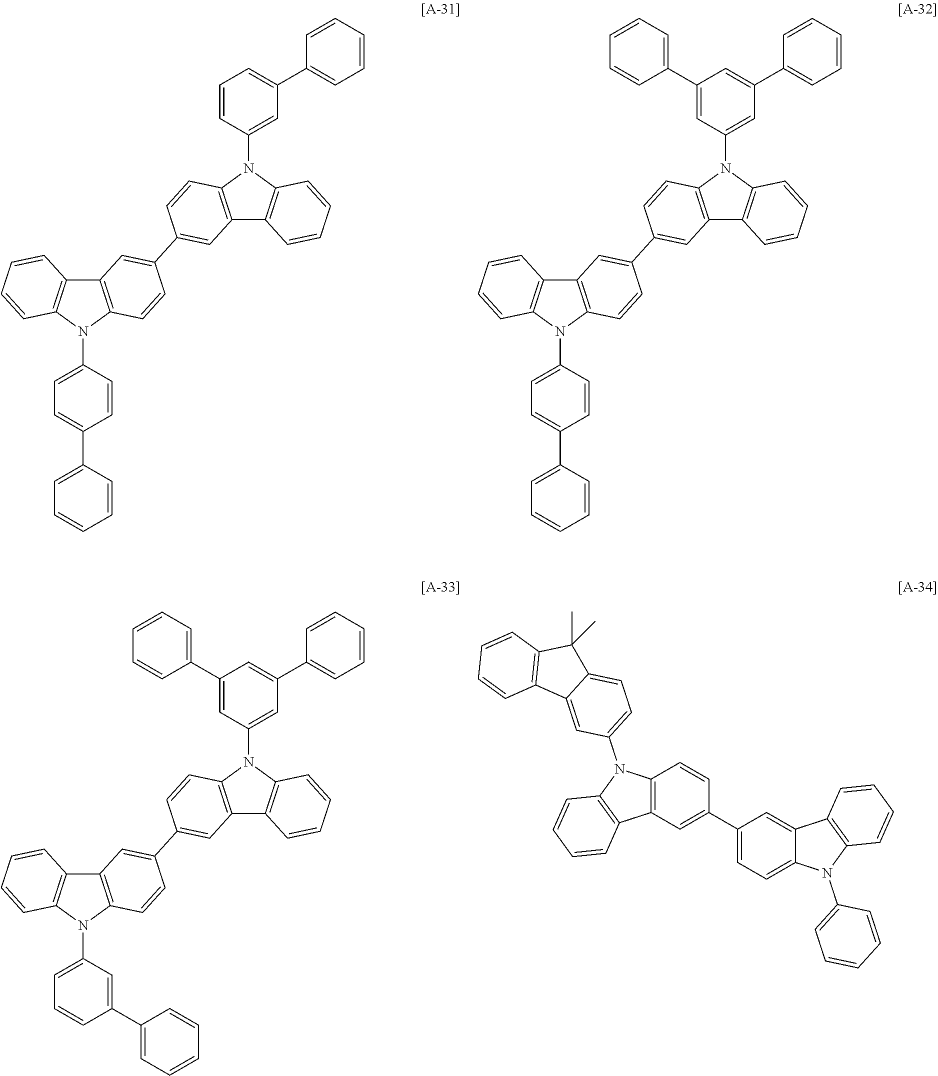 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21]

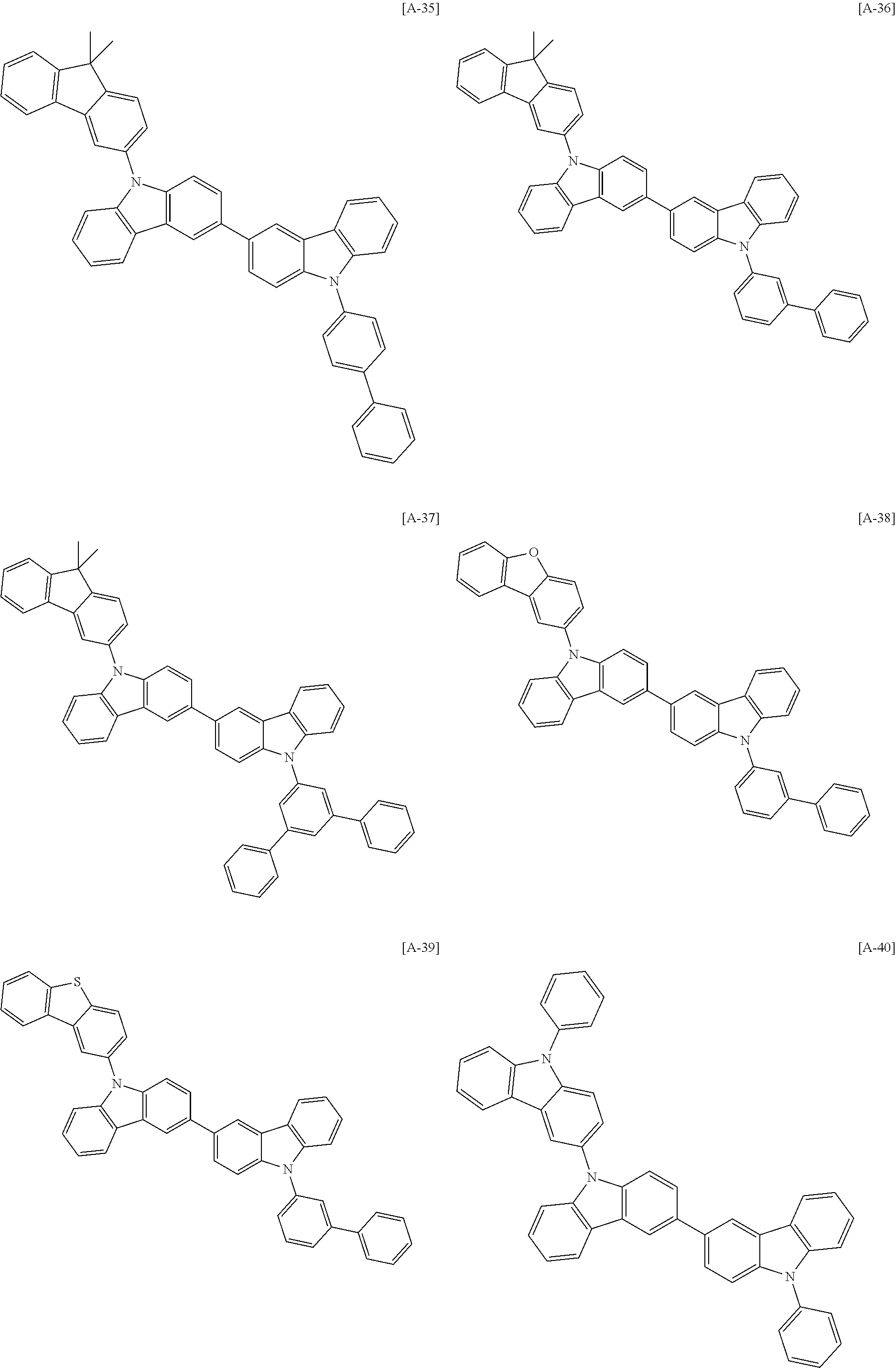

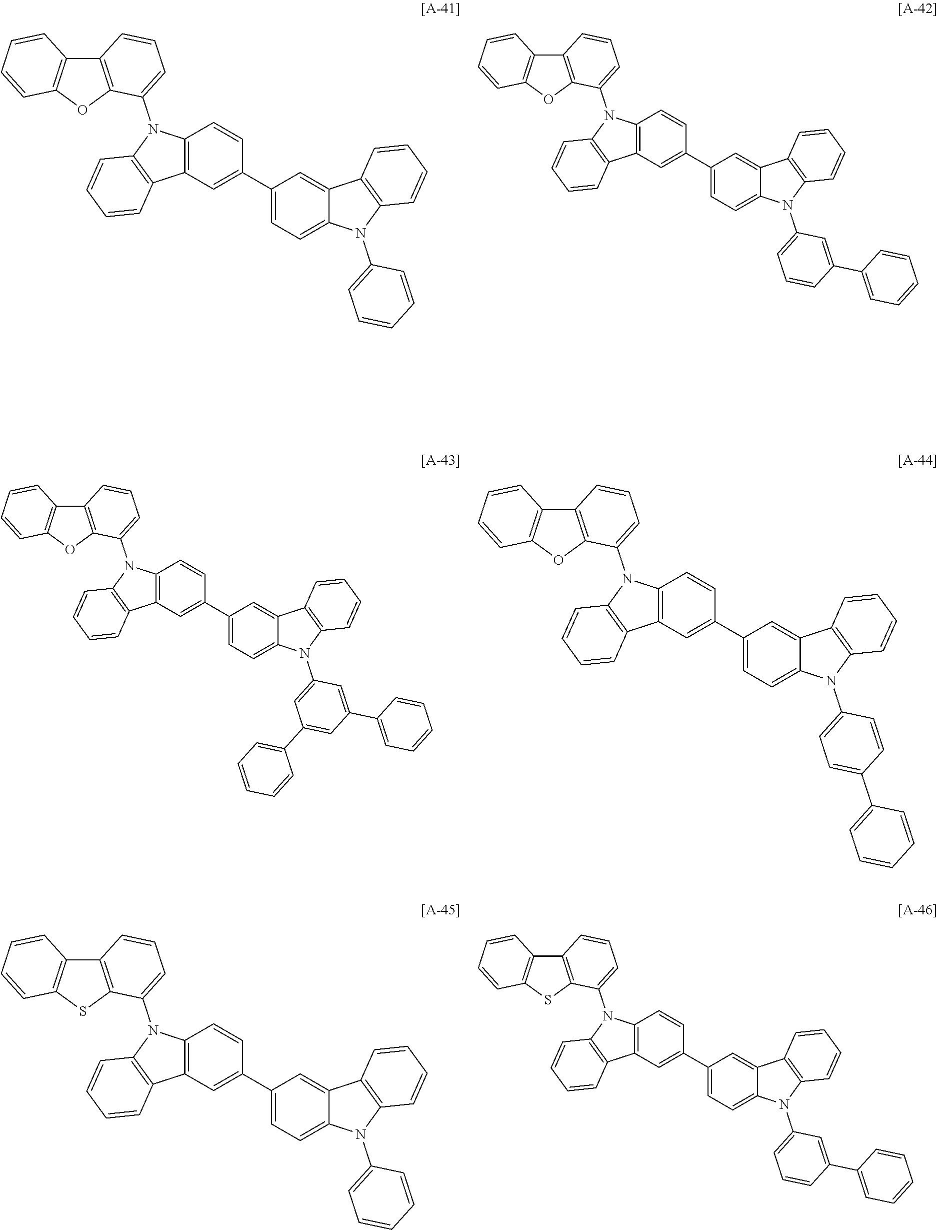

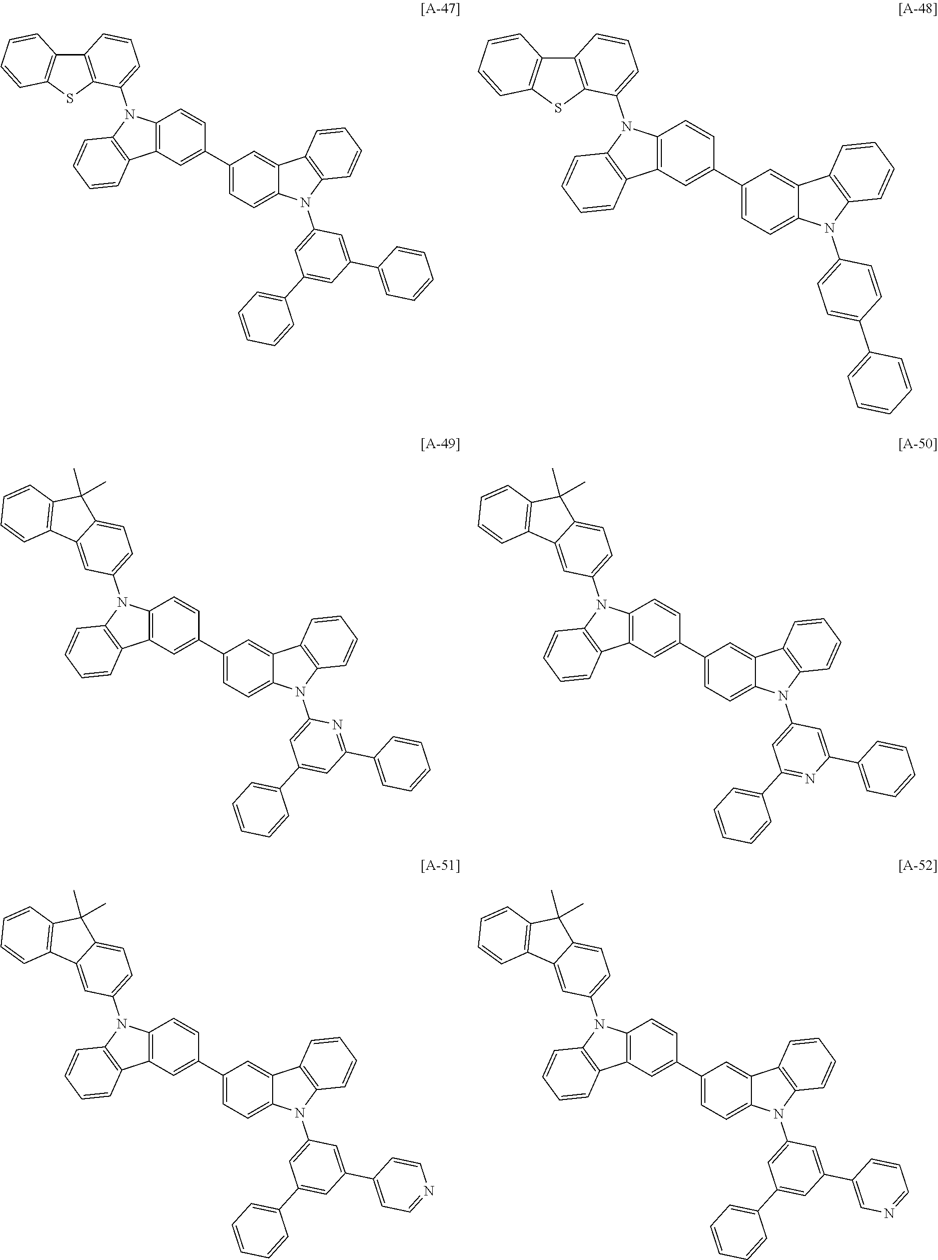

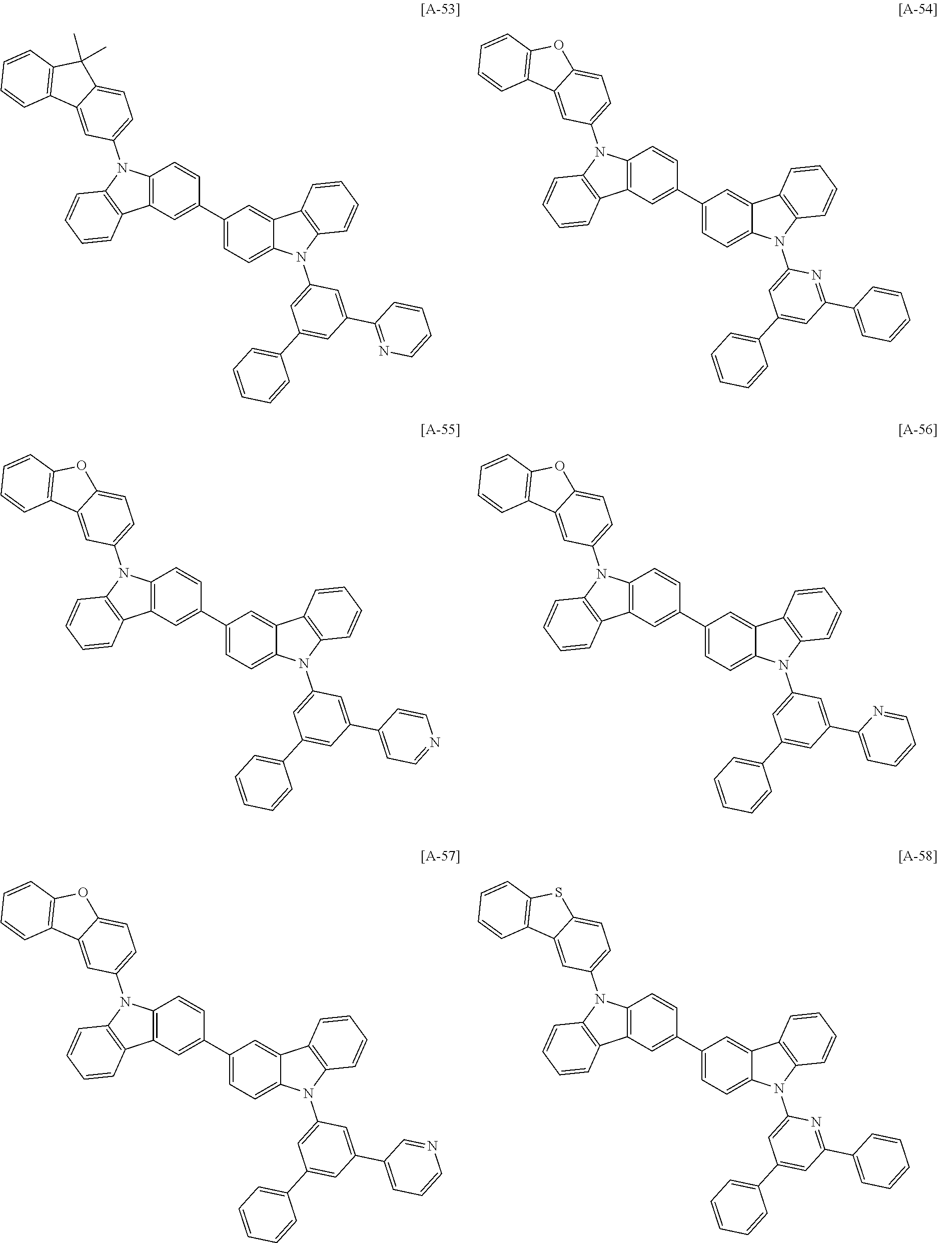

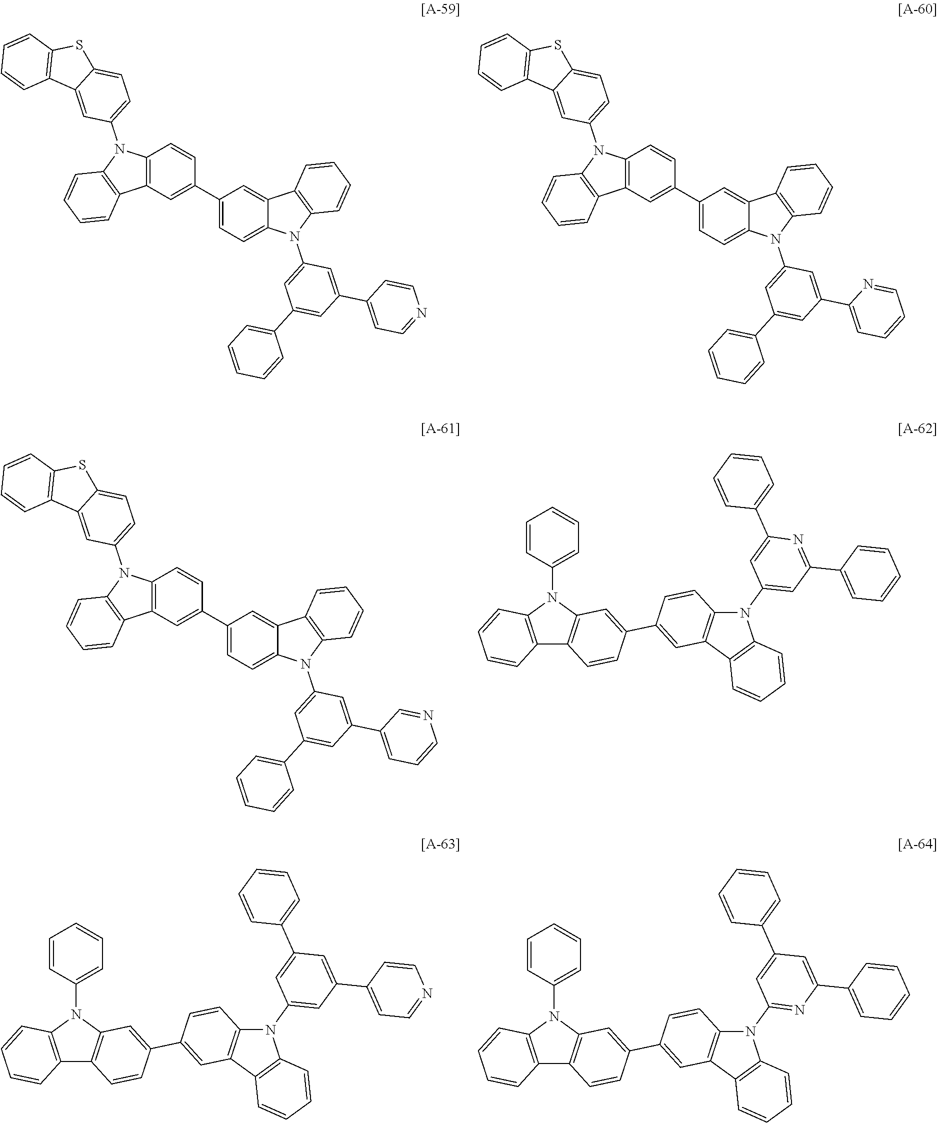

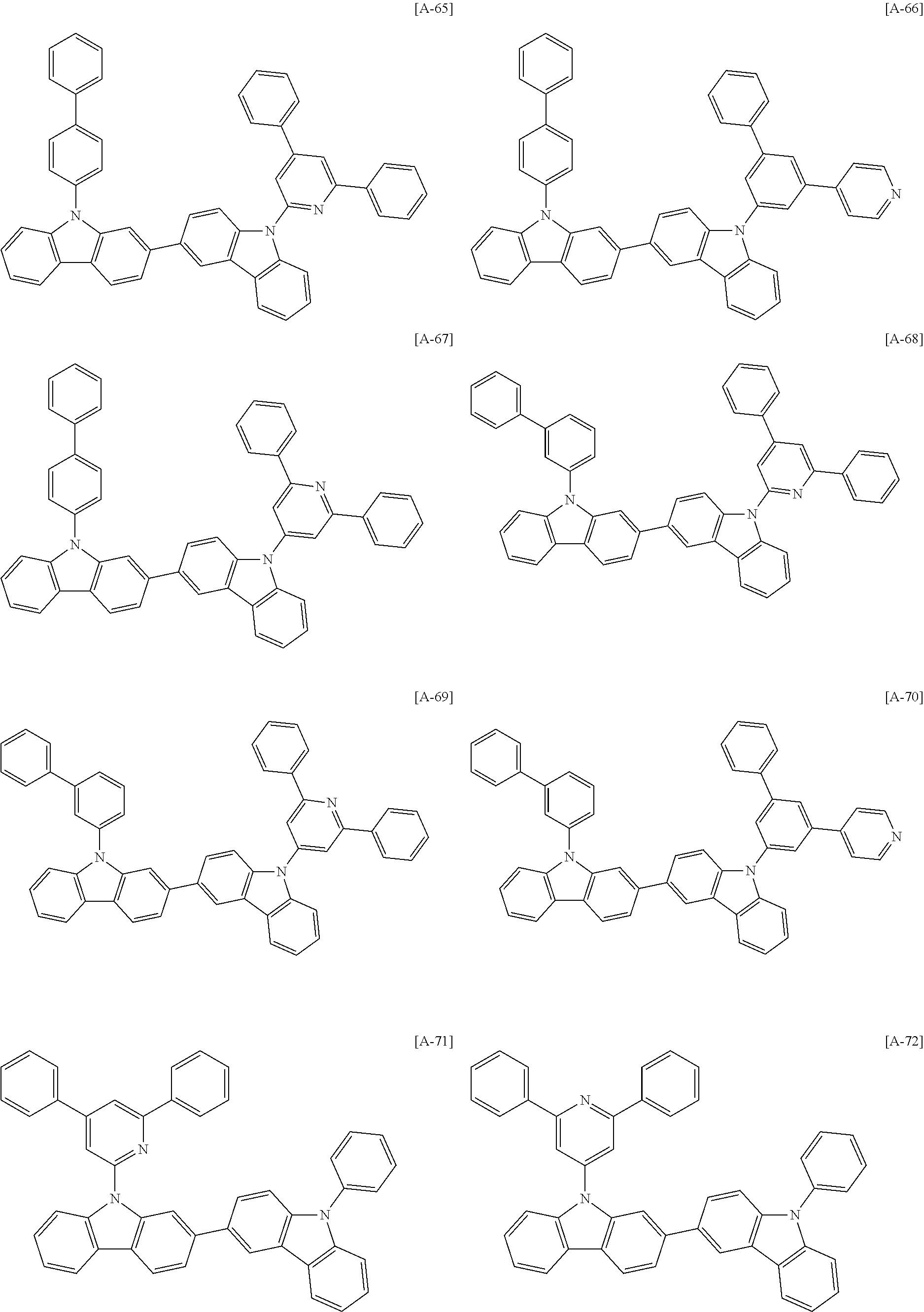

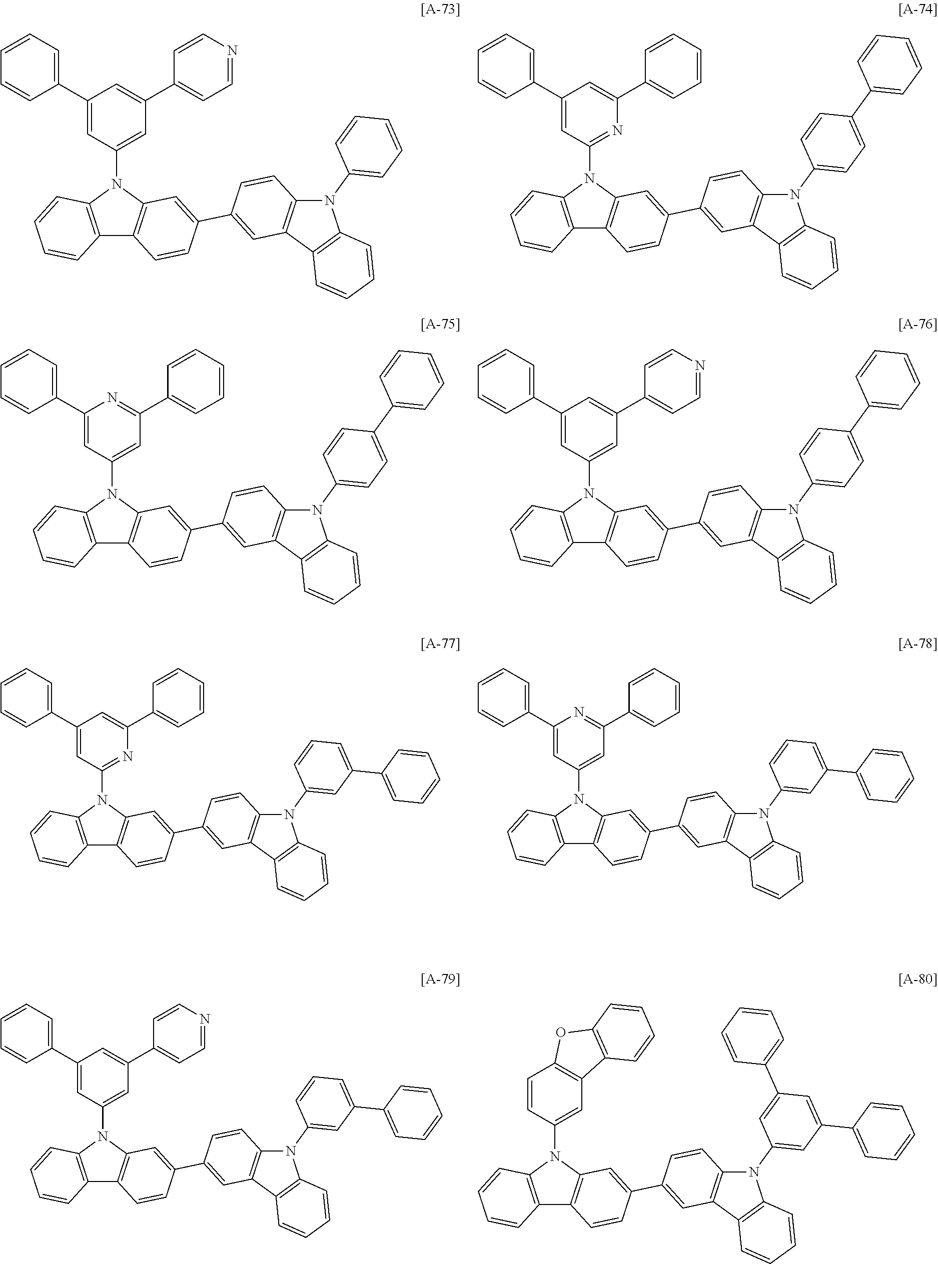

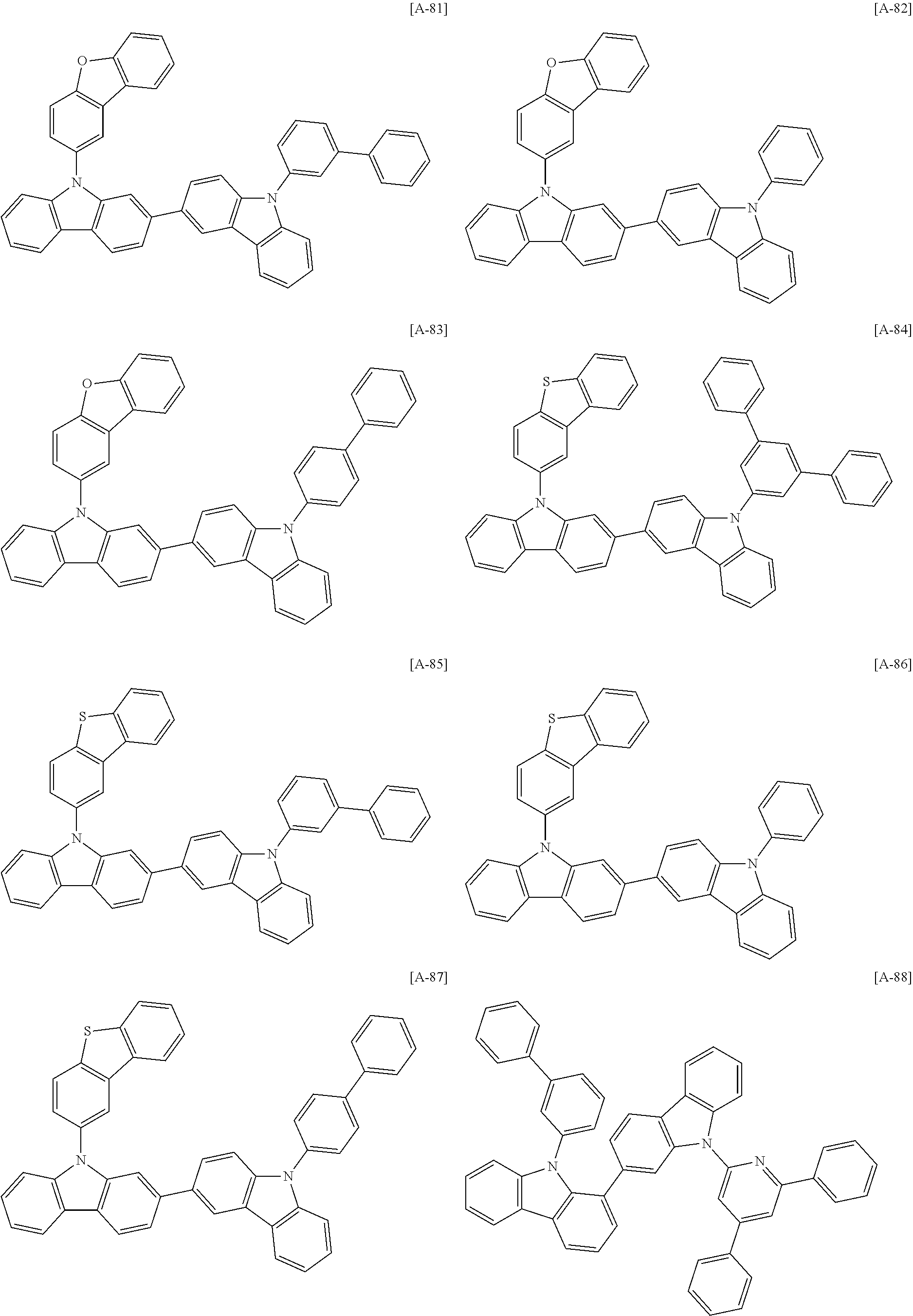

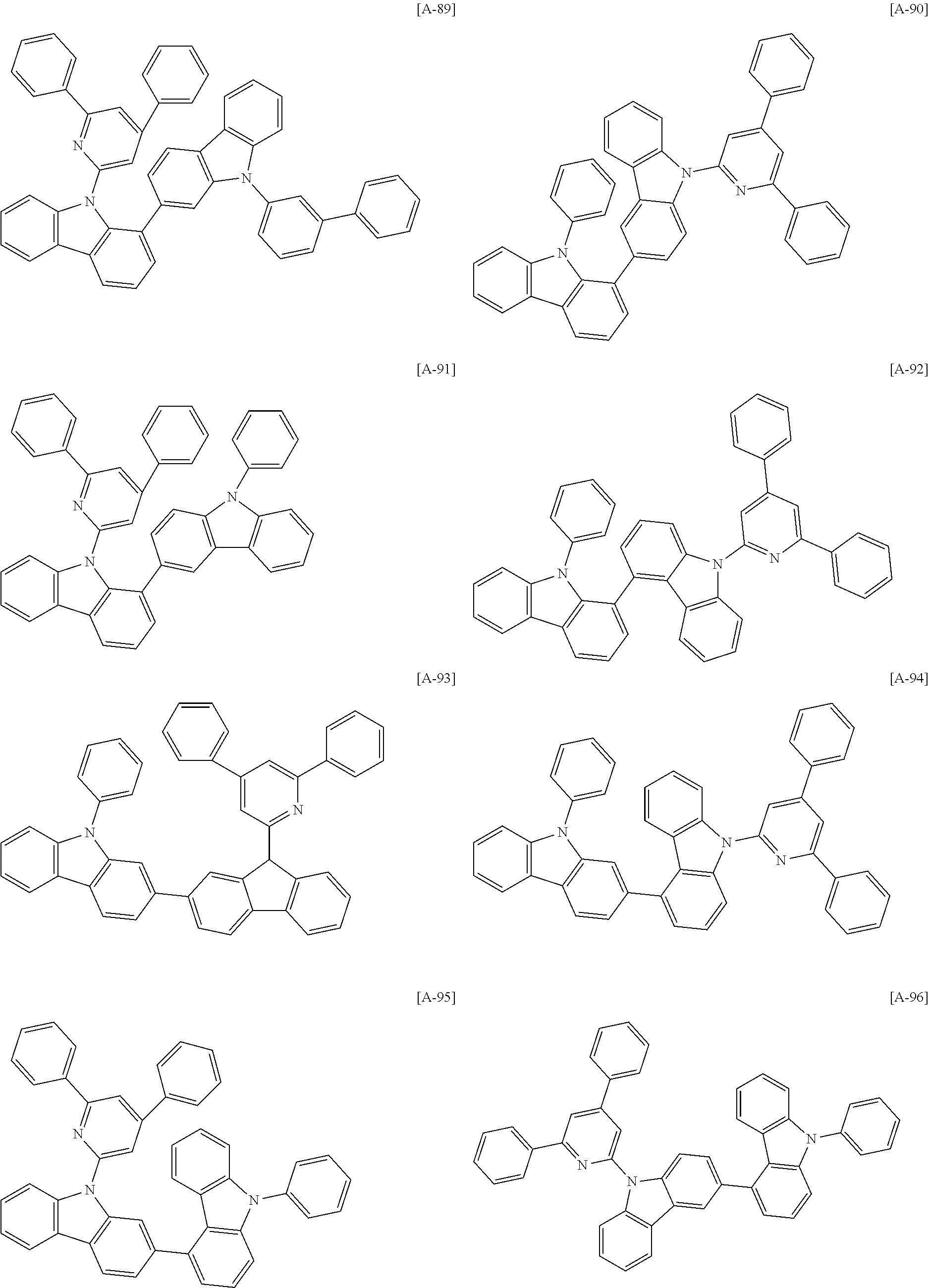

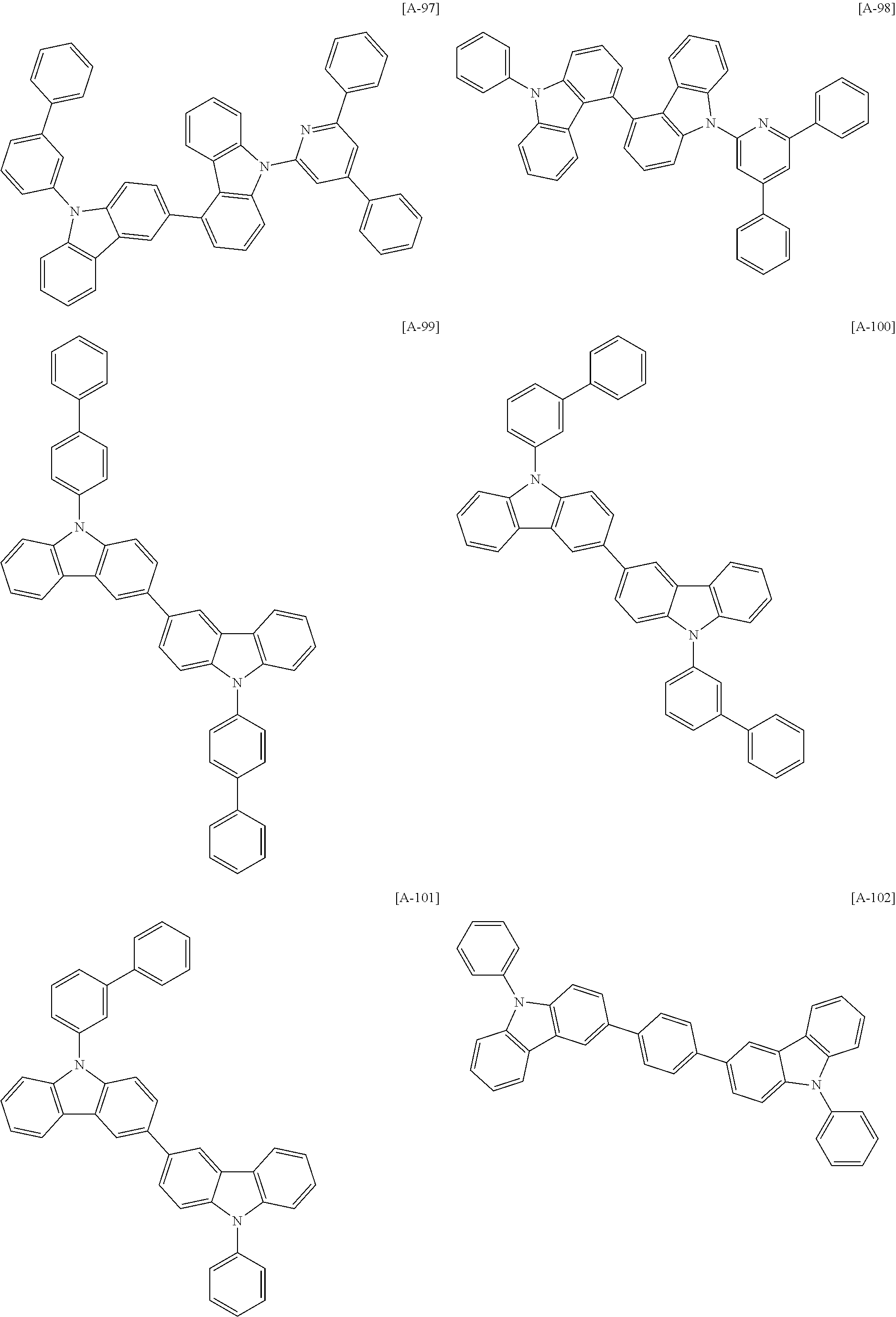

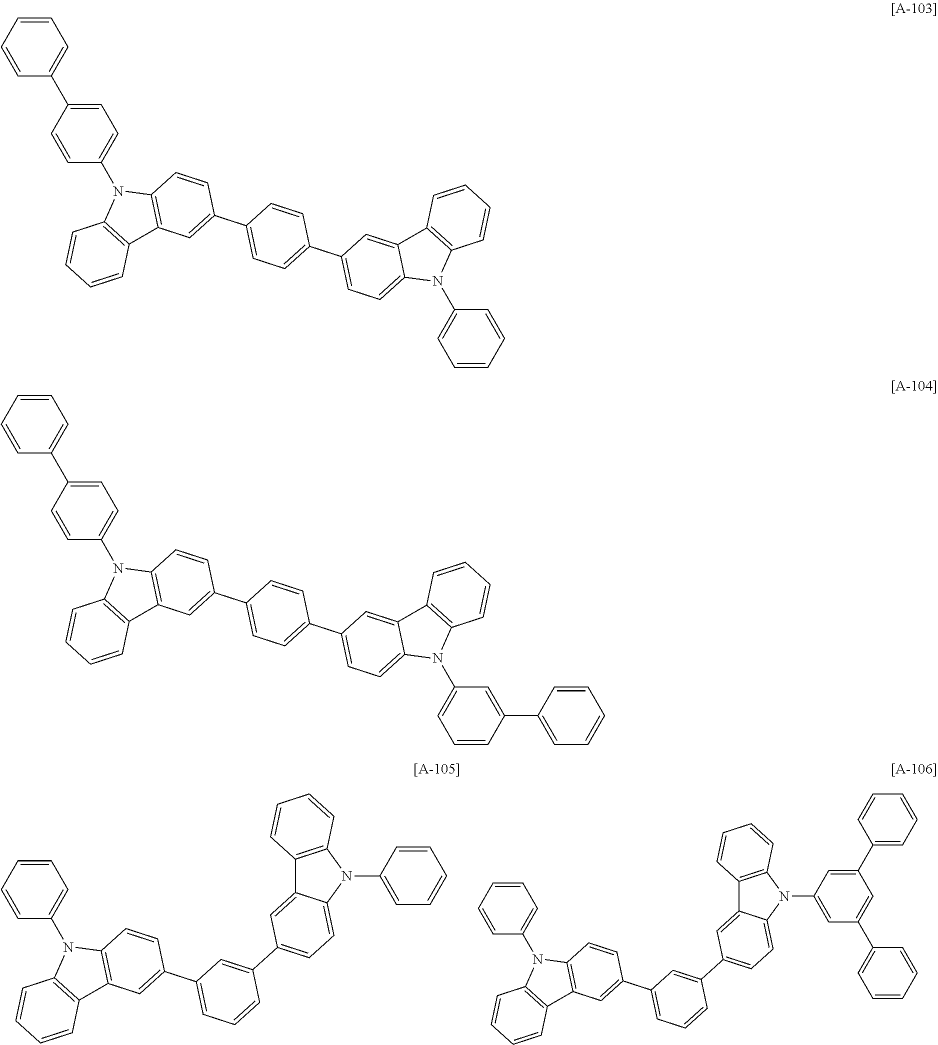

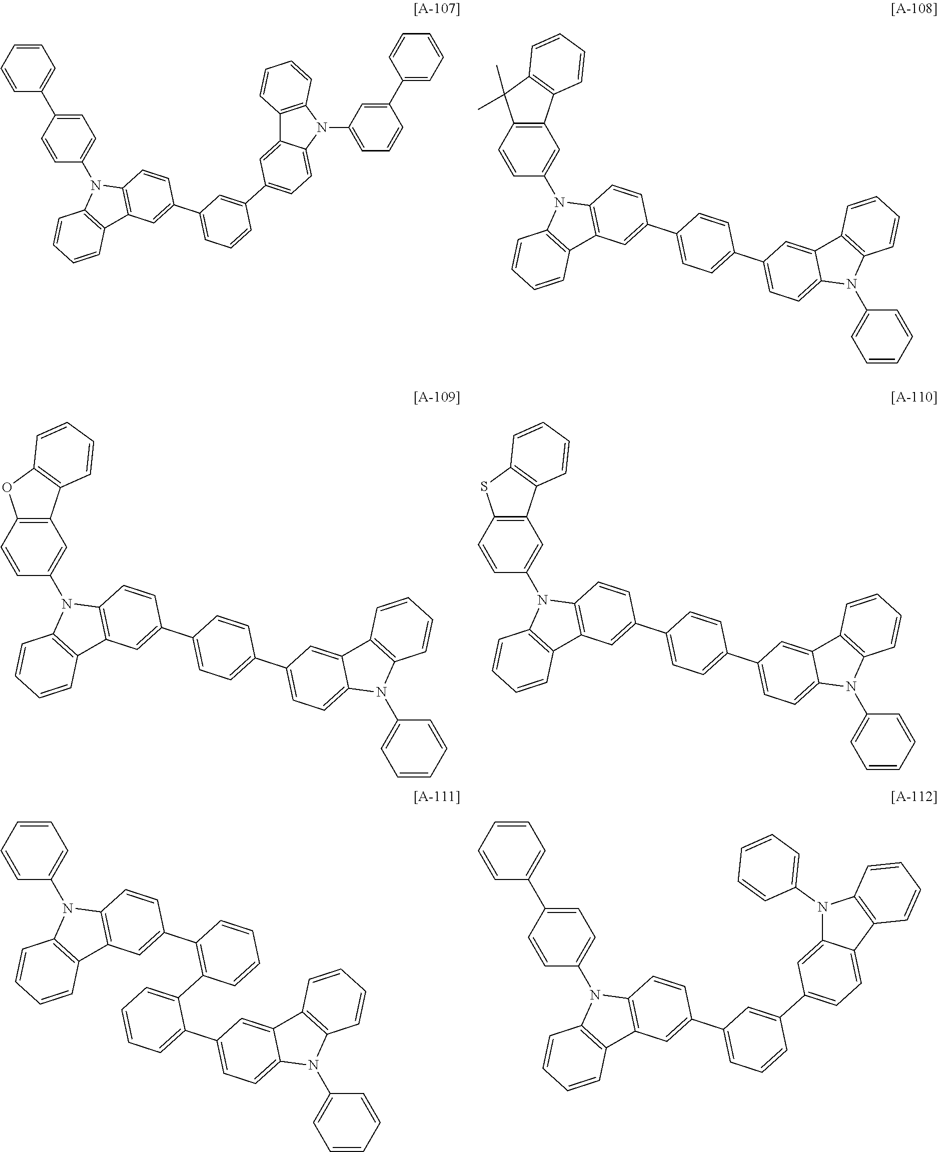

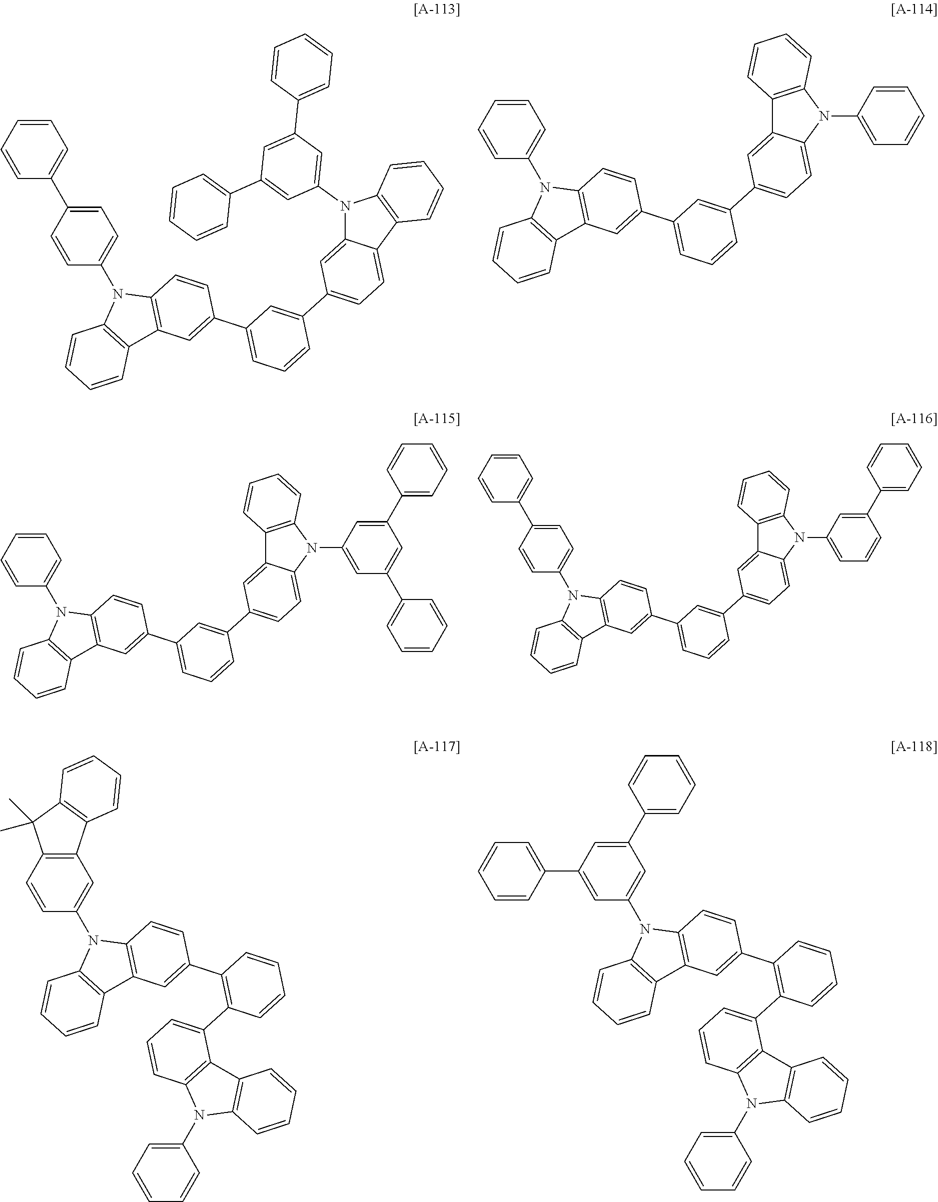

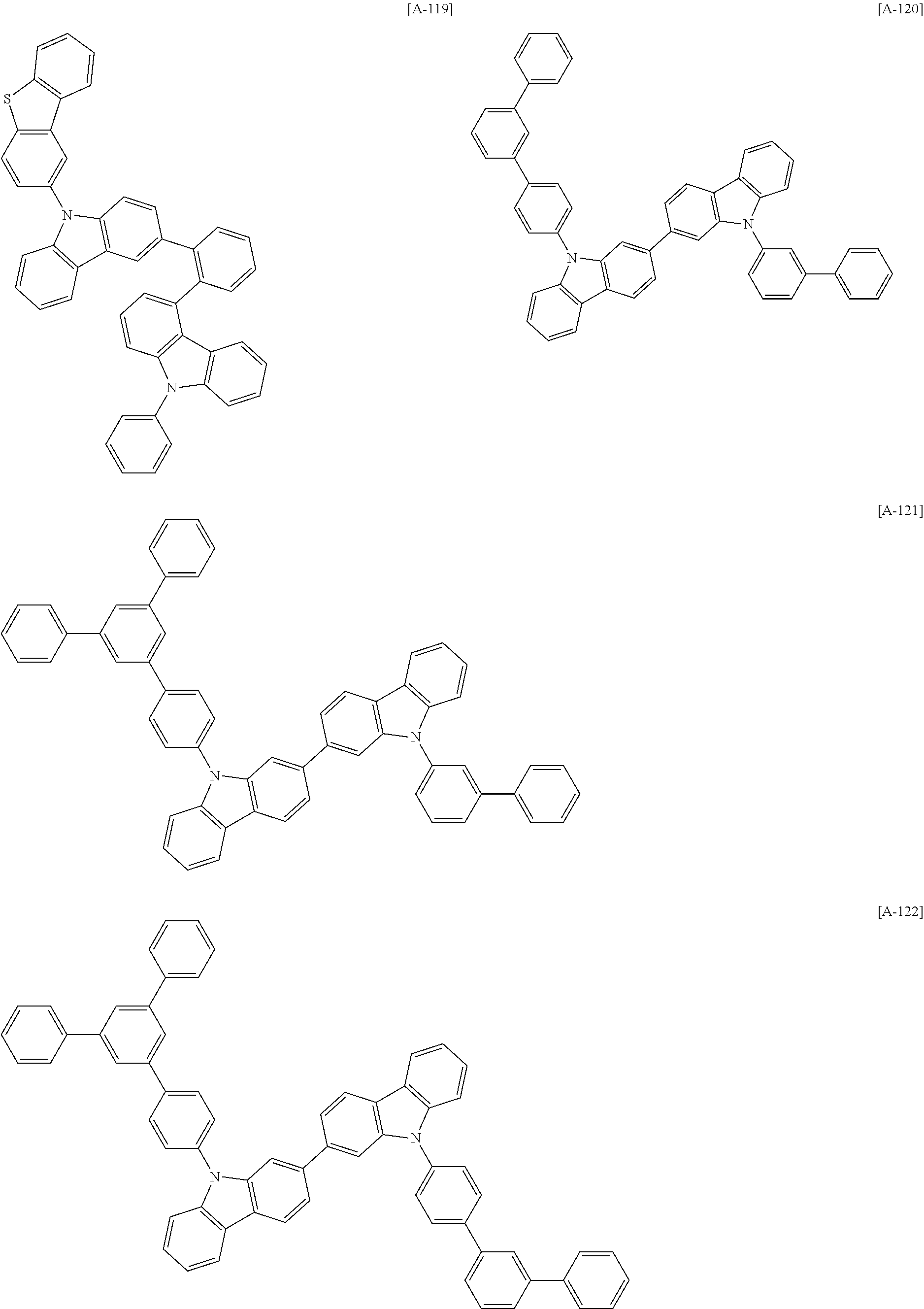 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5ccc(-c6ccc7c8ccccc8n(-c8ccc(-c9cc(-c%10ccccc%10)cc(-c%10ccccc%10)c9)cc8)c7c6)cc54)cc3)c2)cc1]

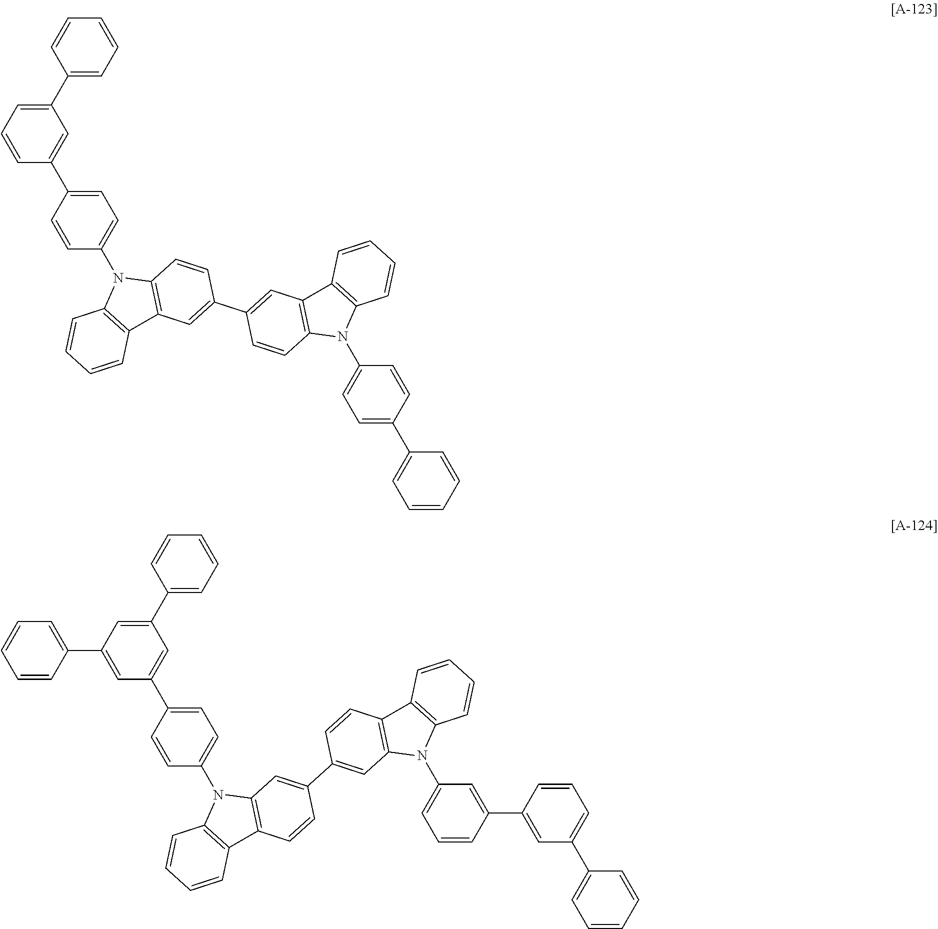 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

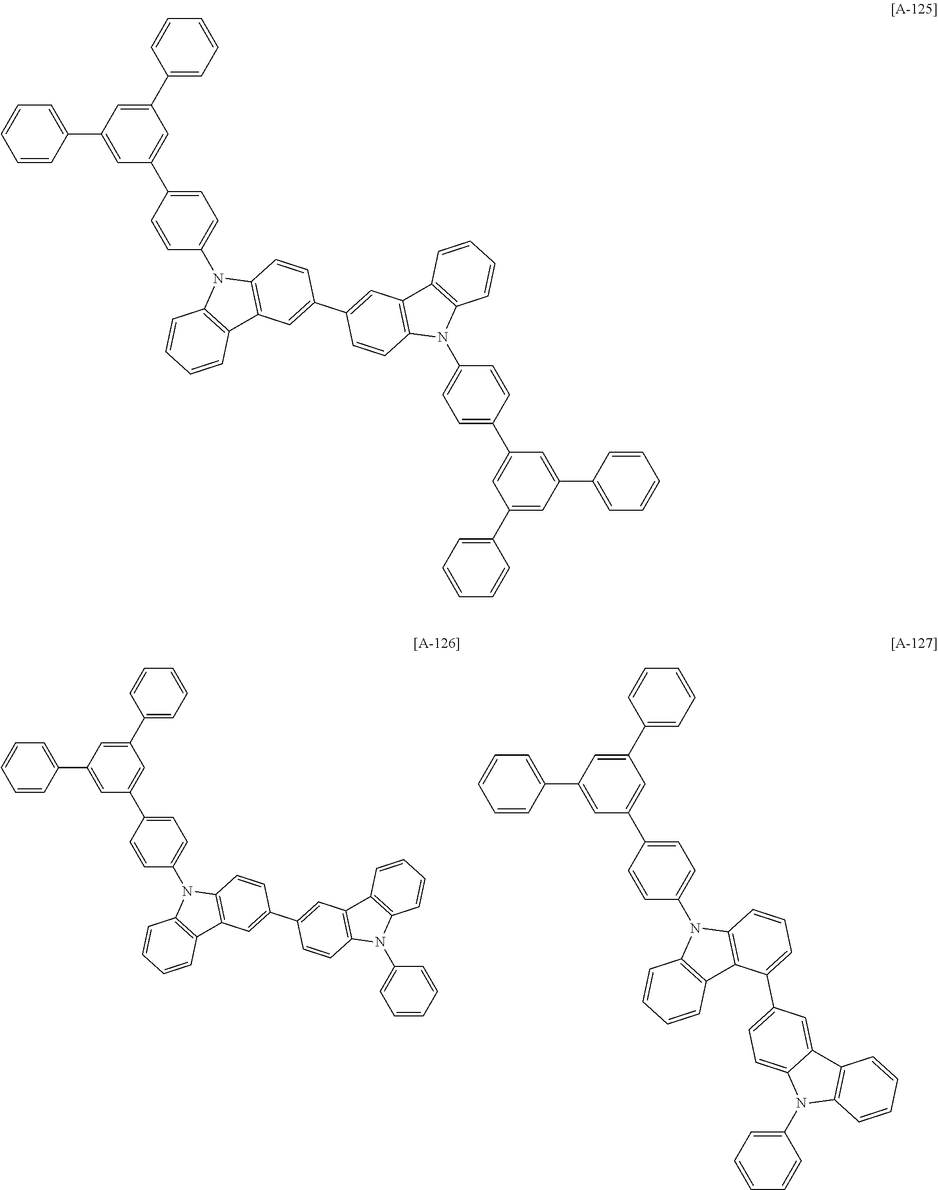 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

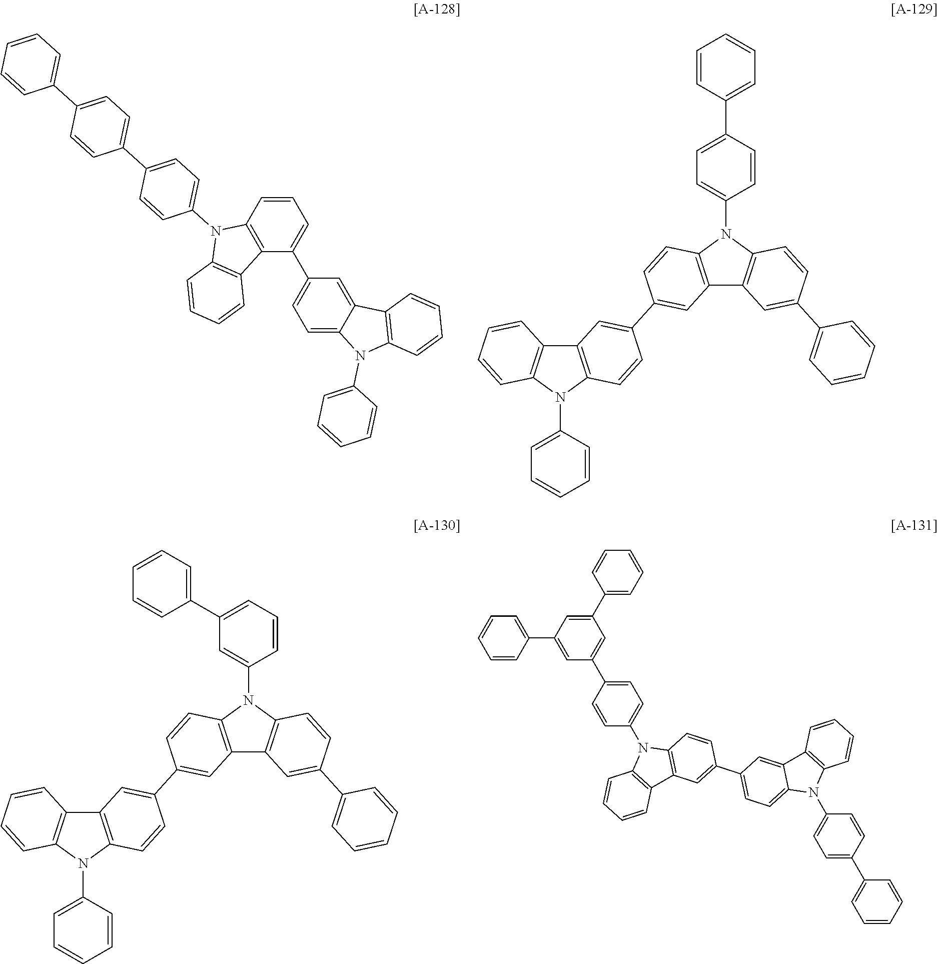

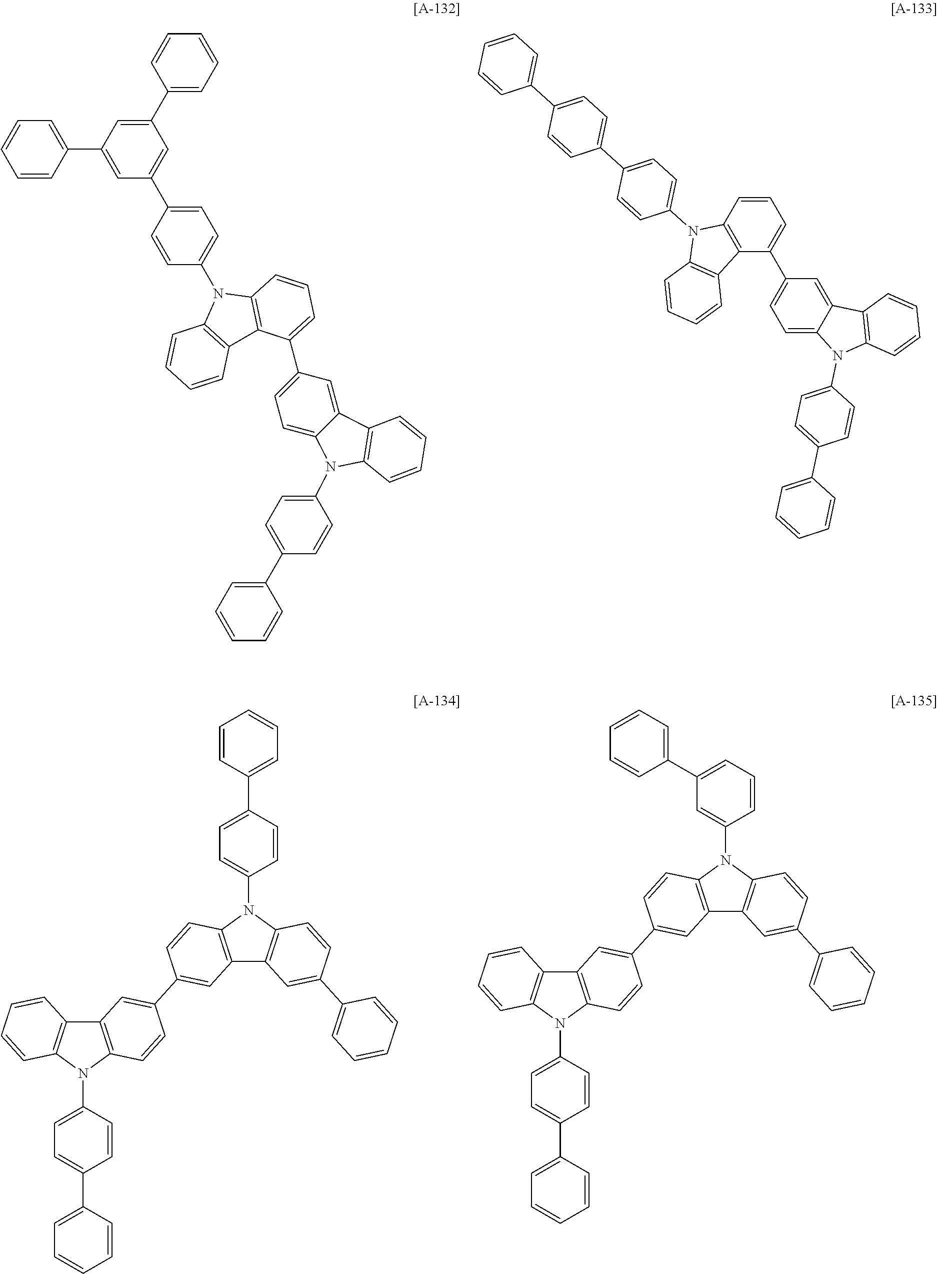 [SMILES: c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1]

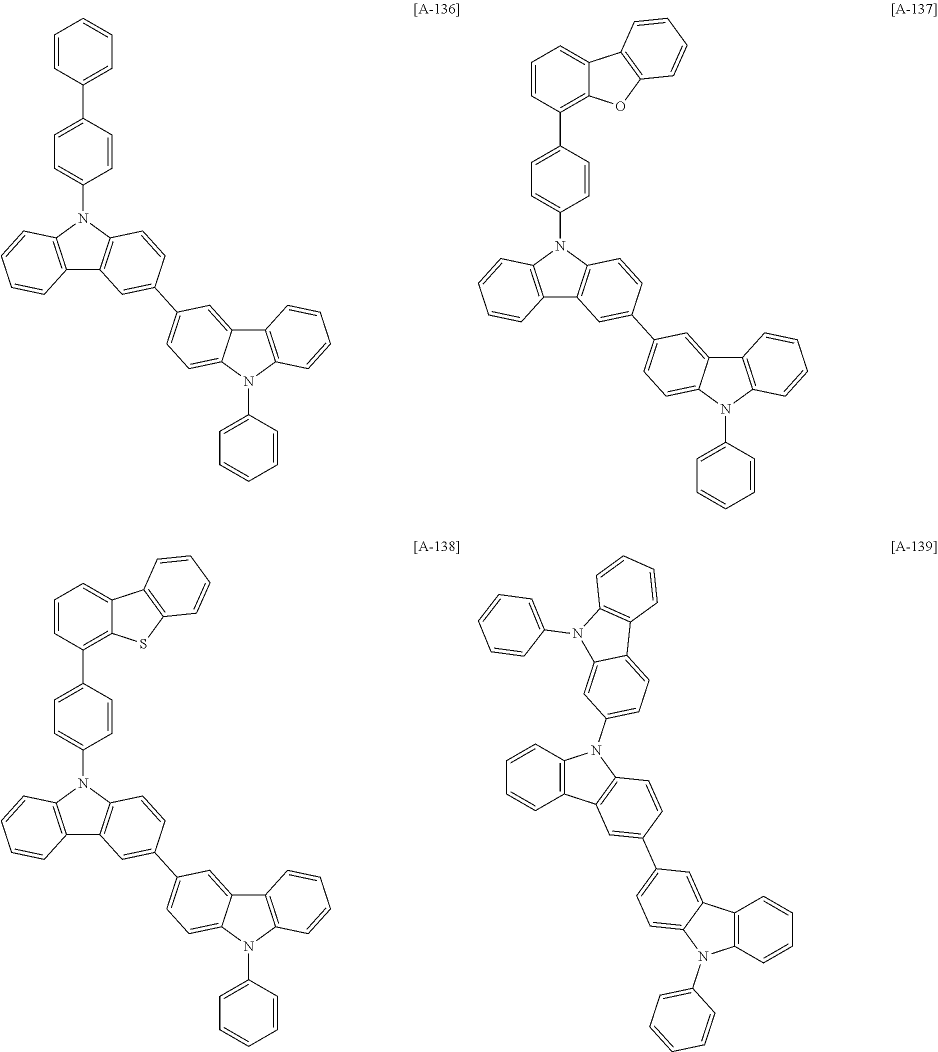 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

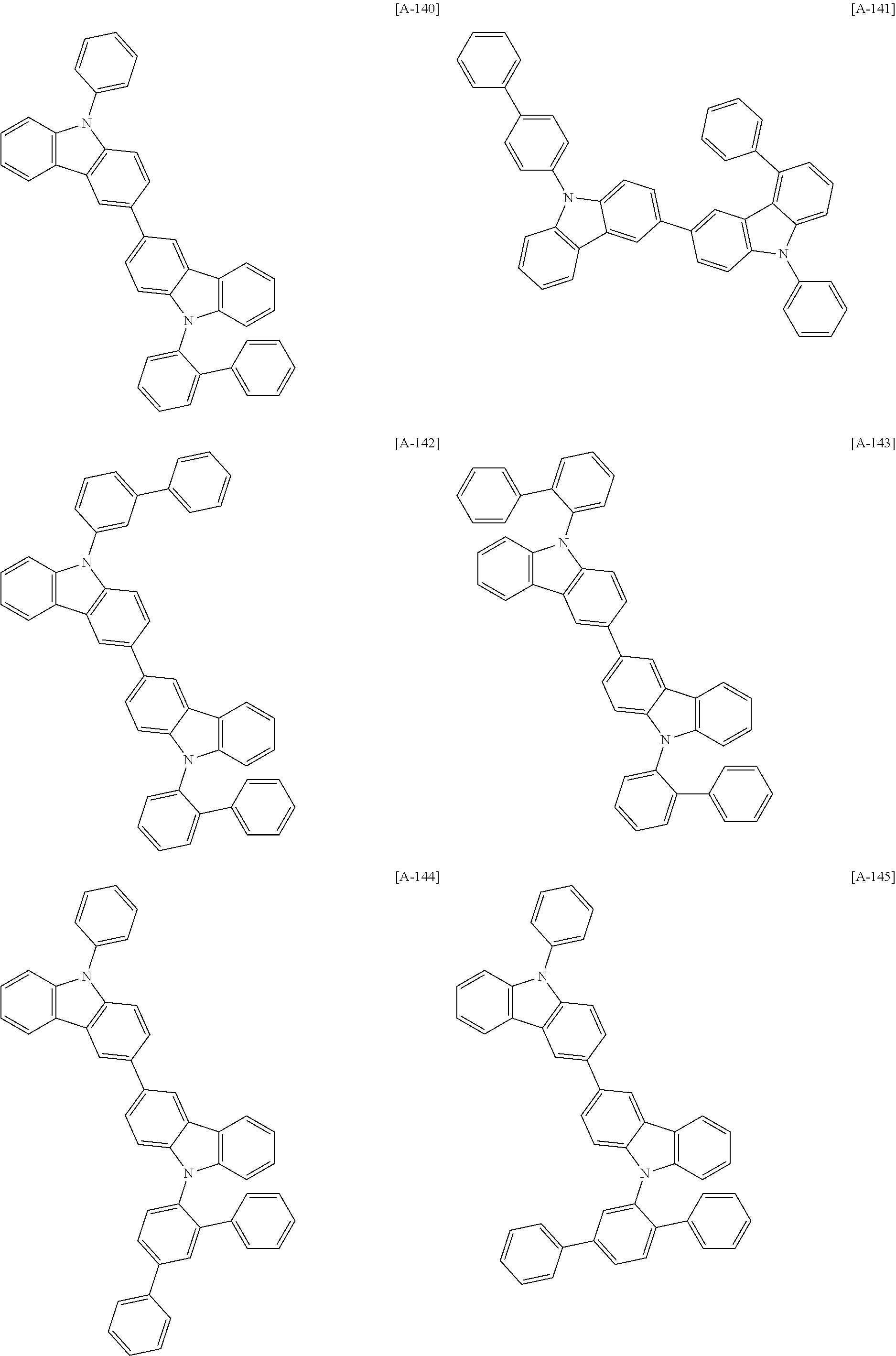

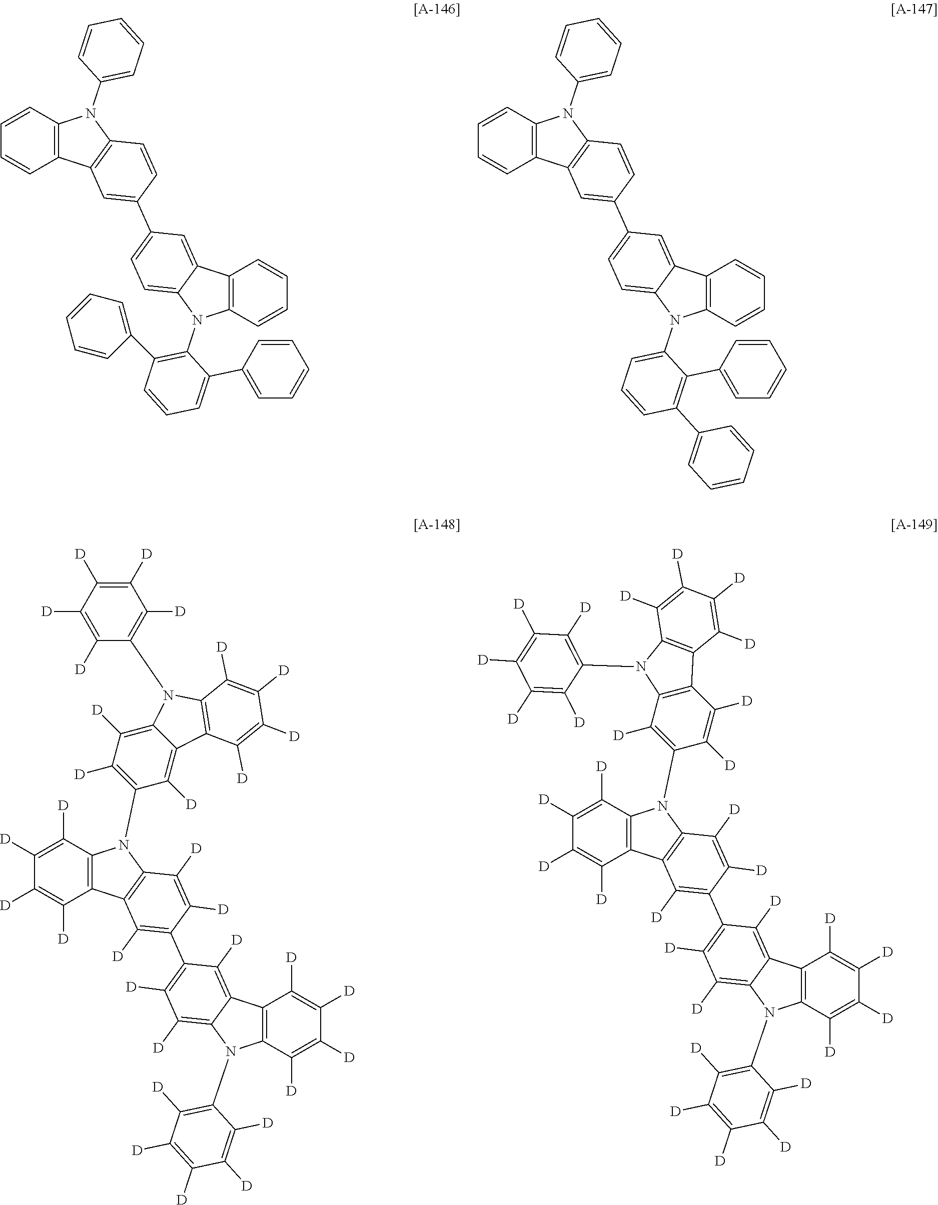

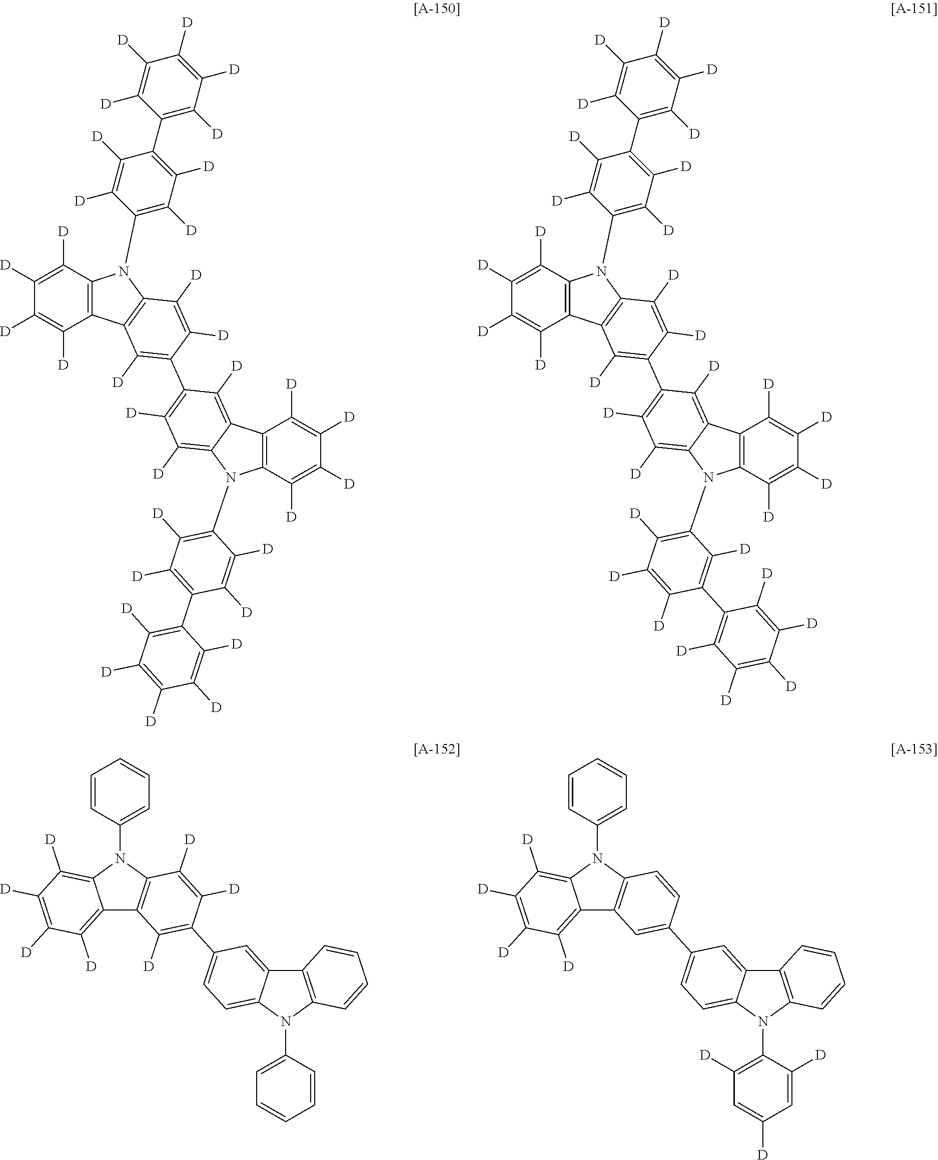

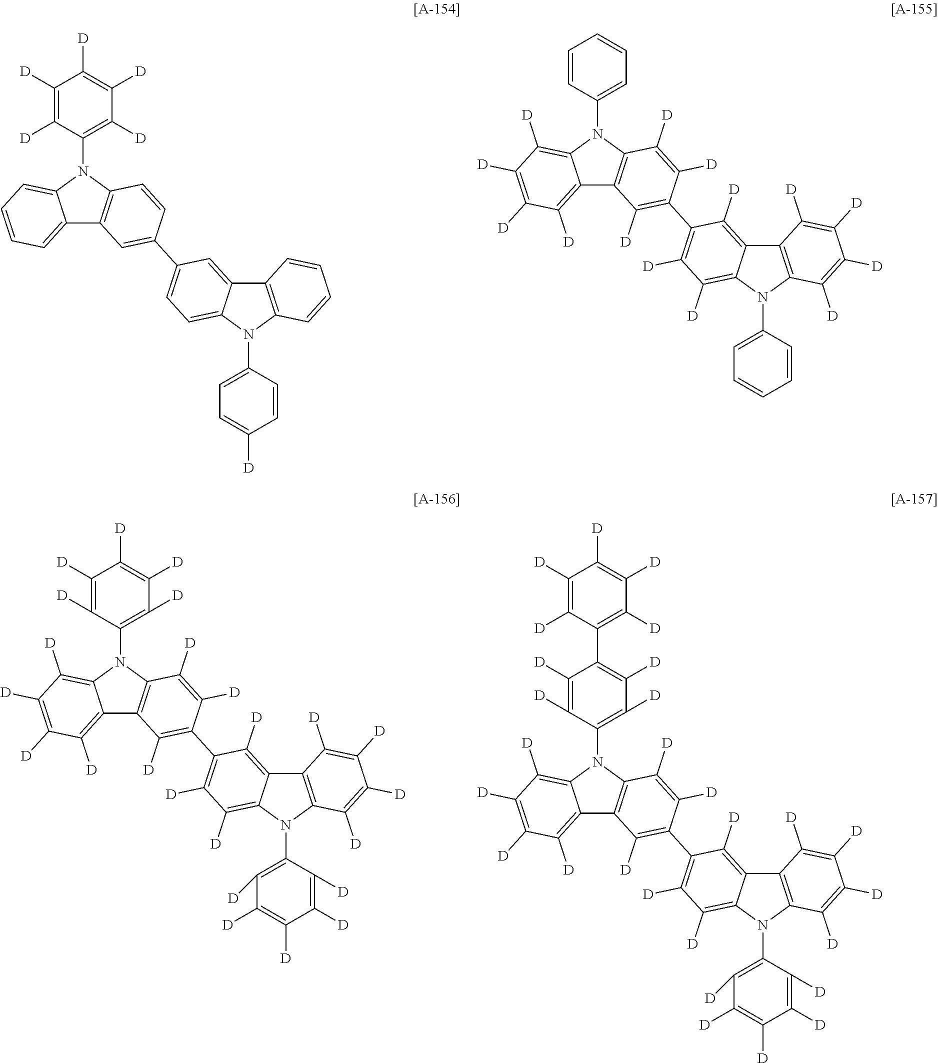

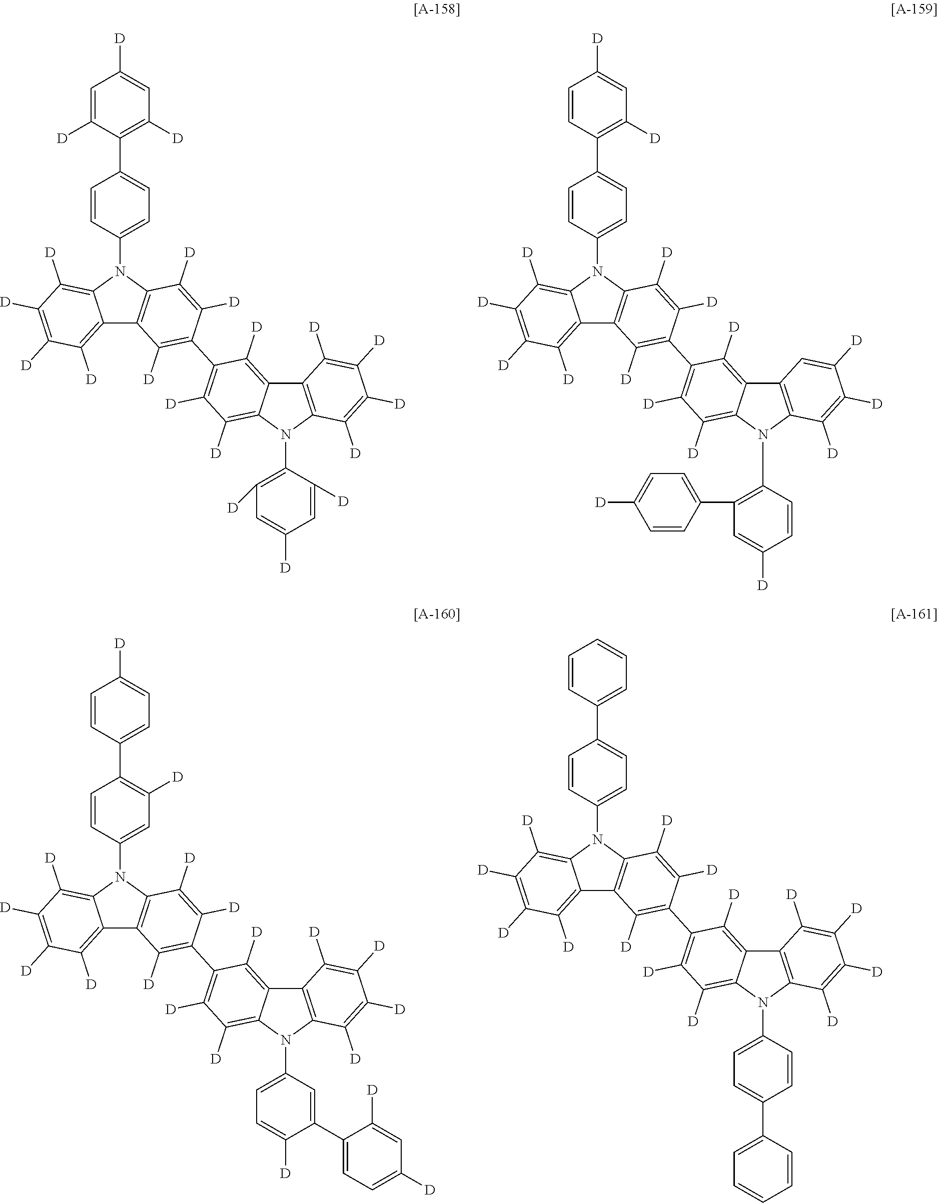

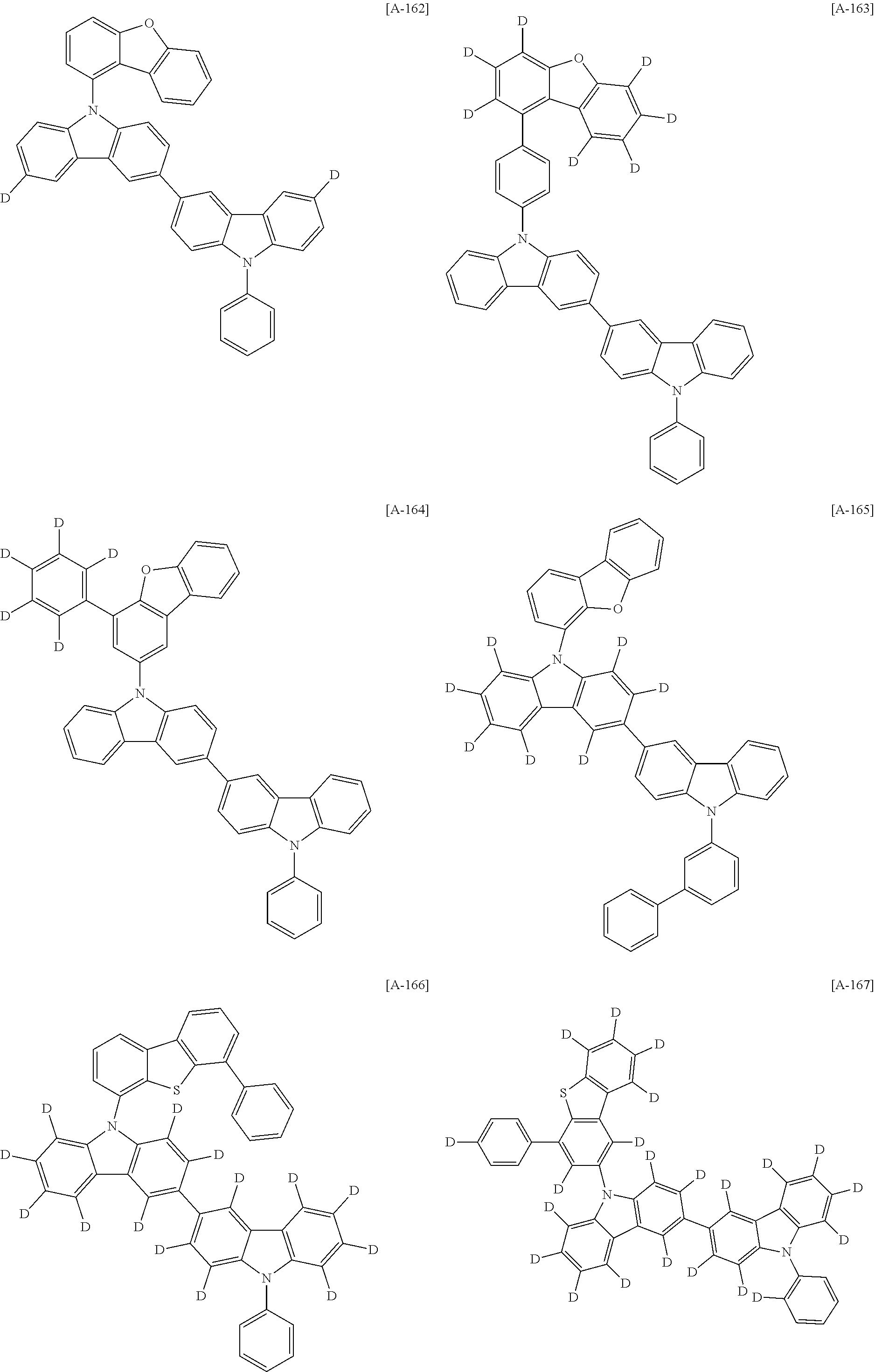

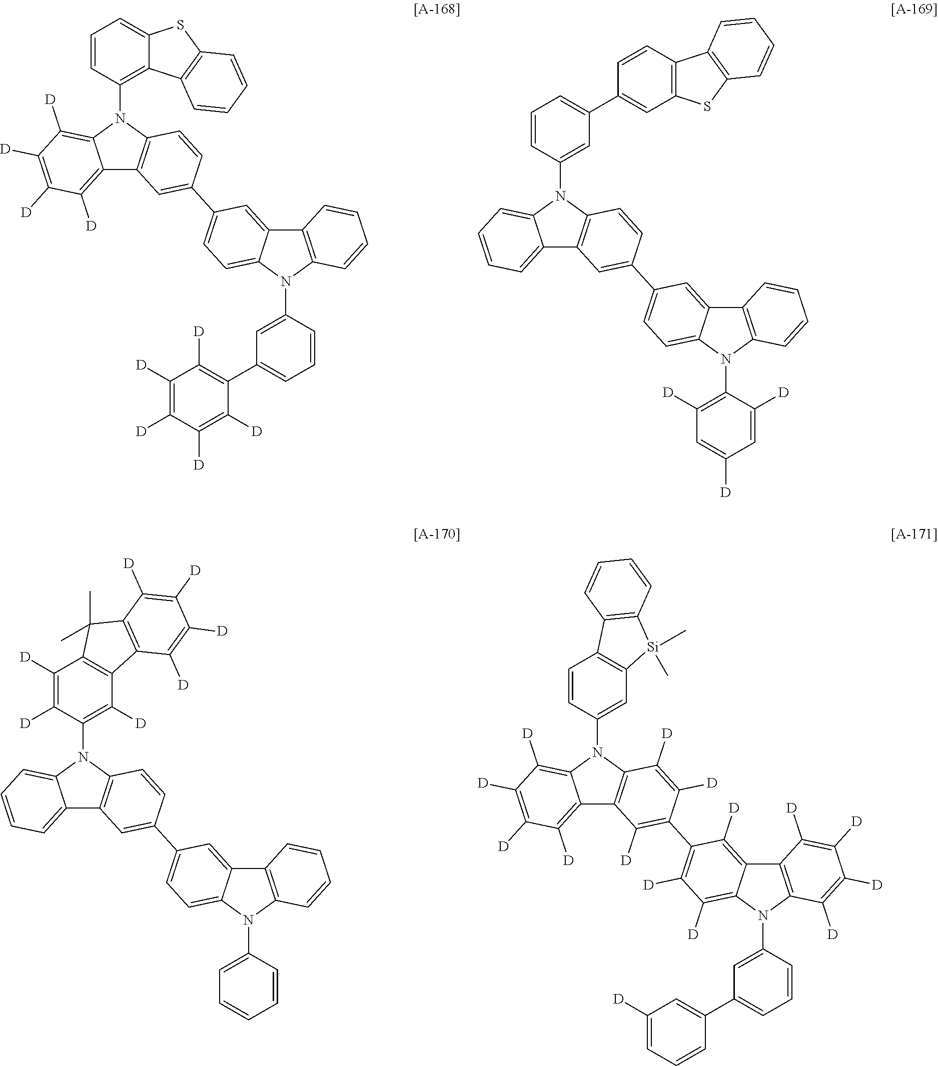

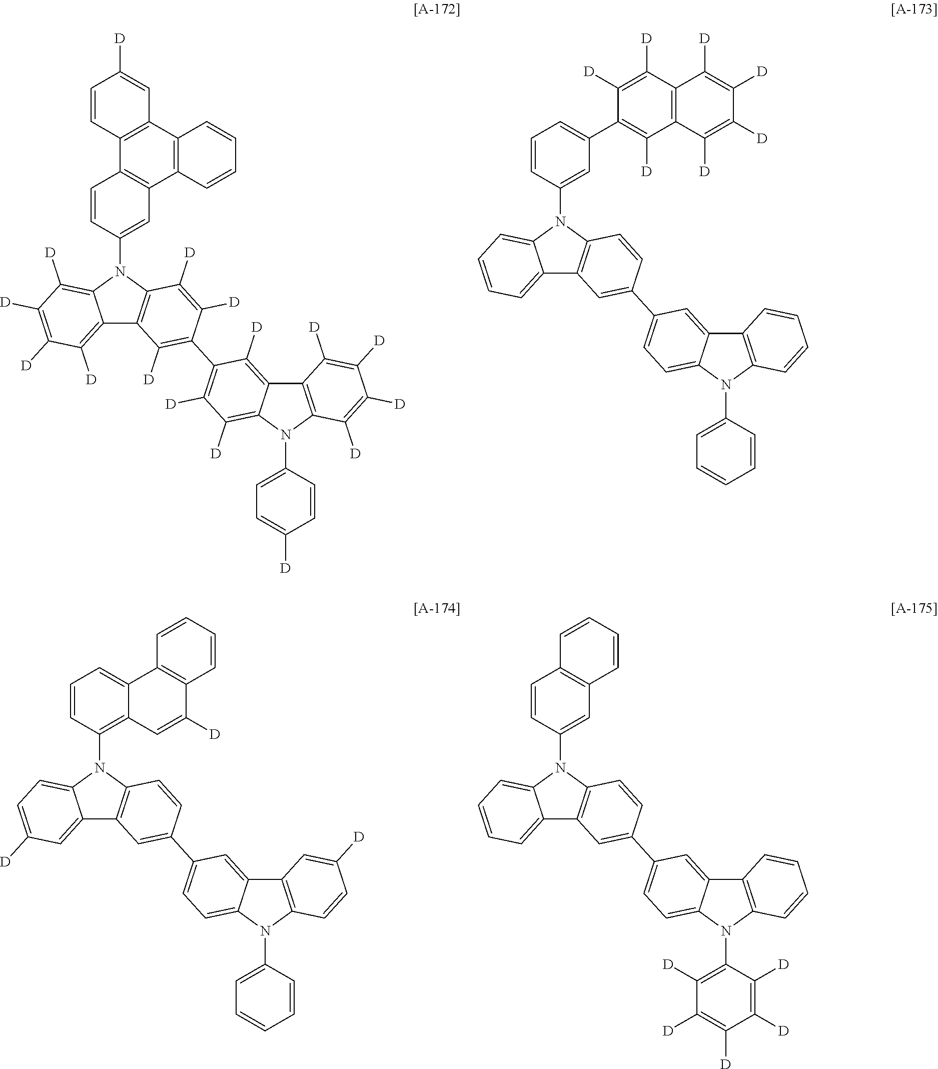 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

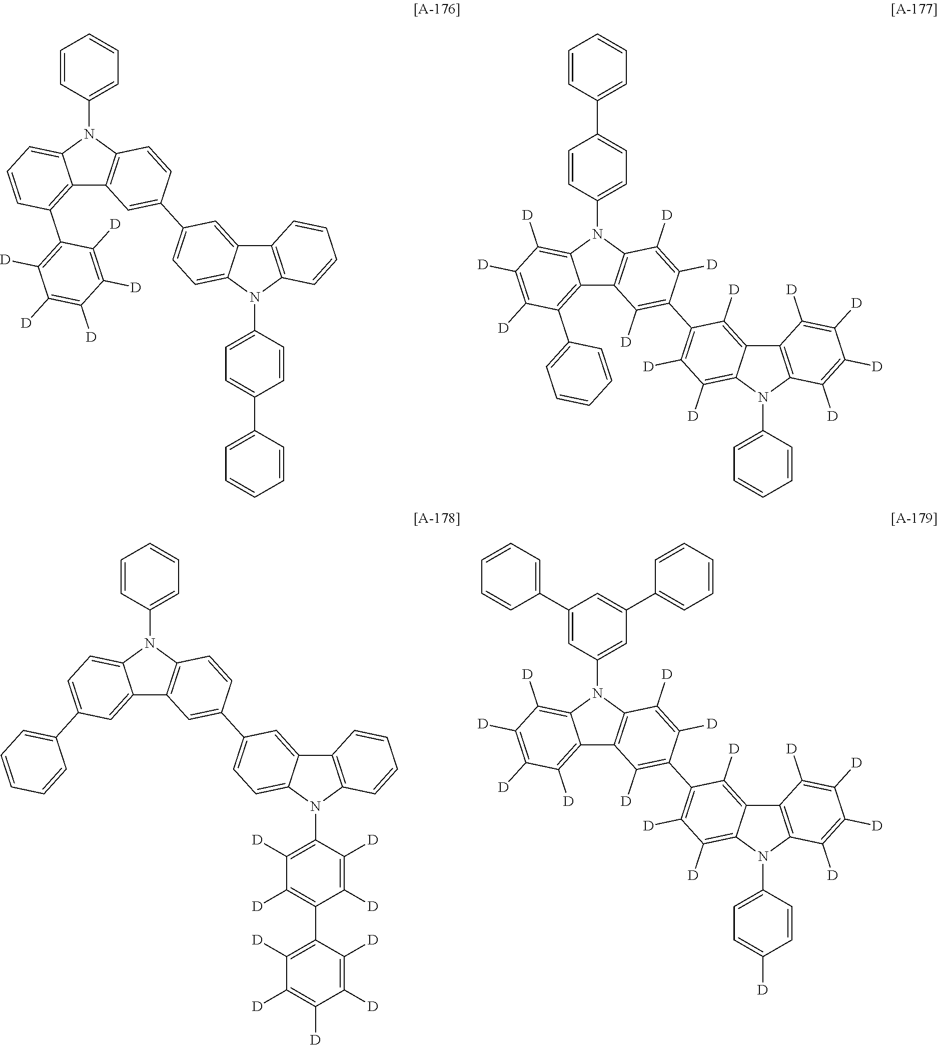

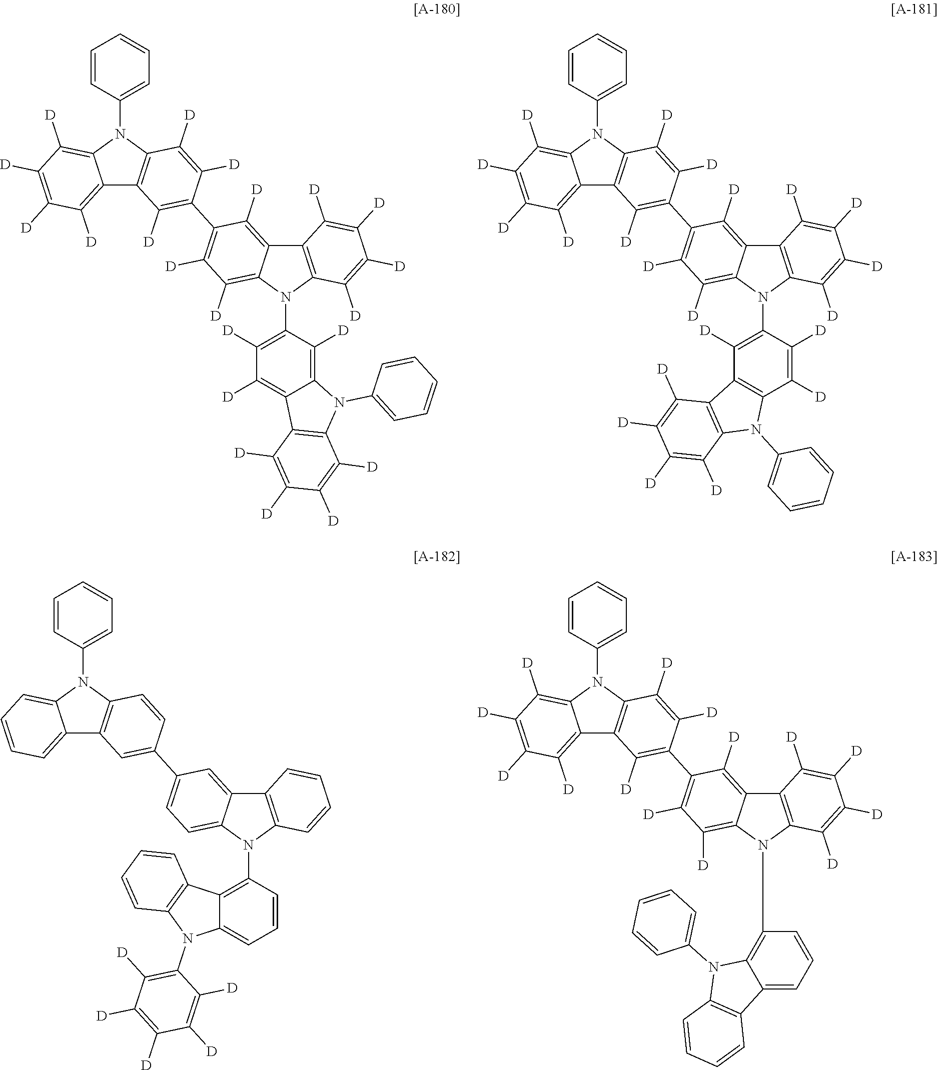

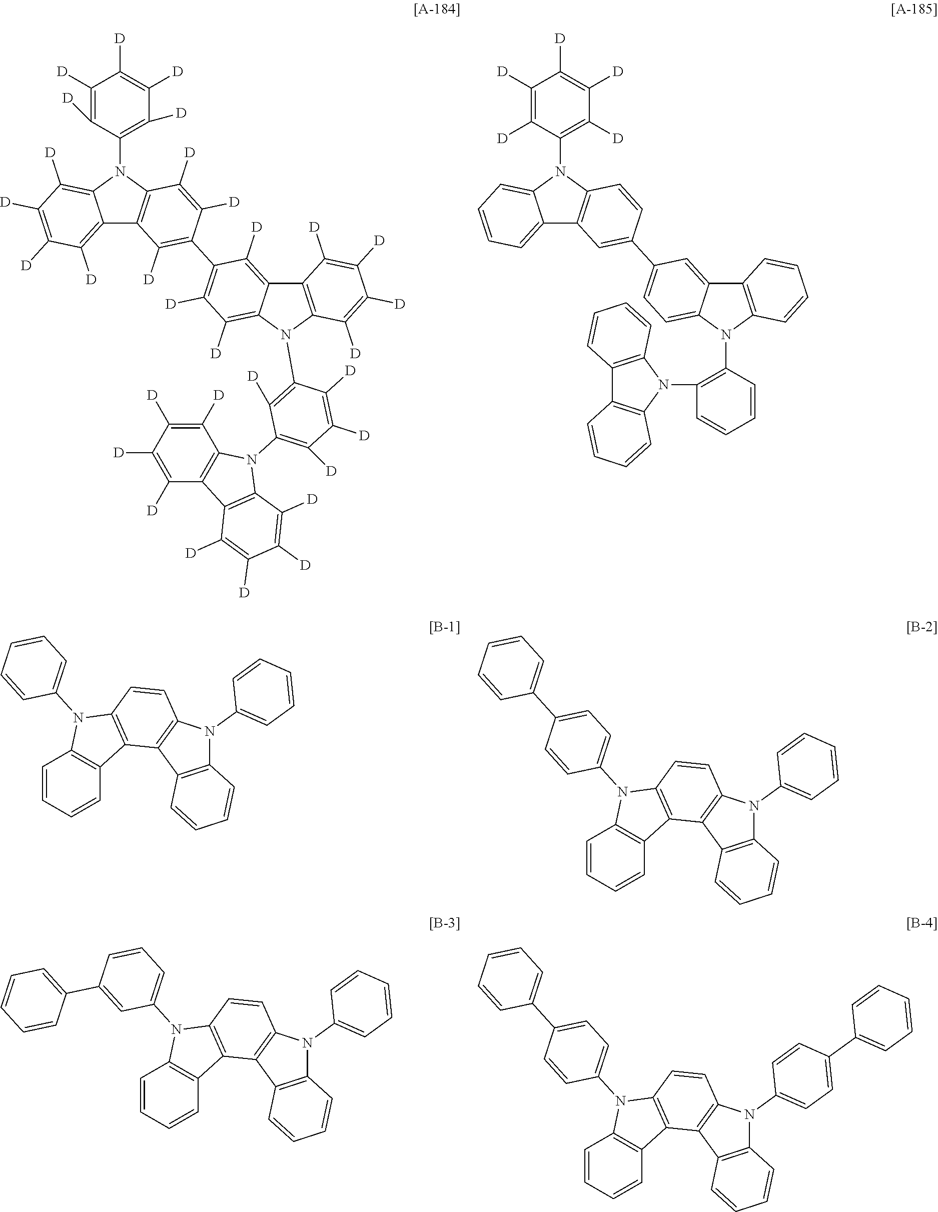

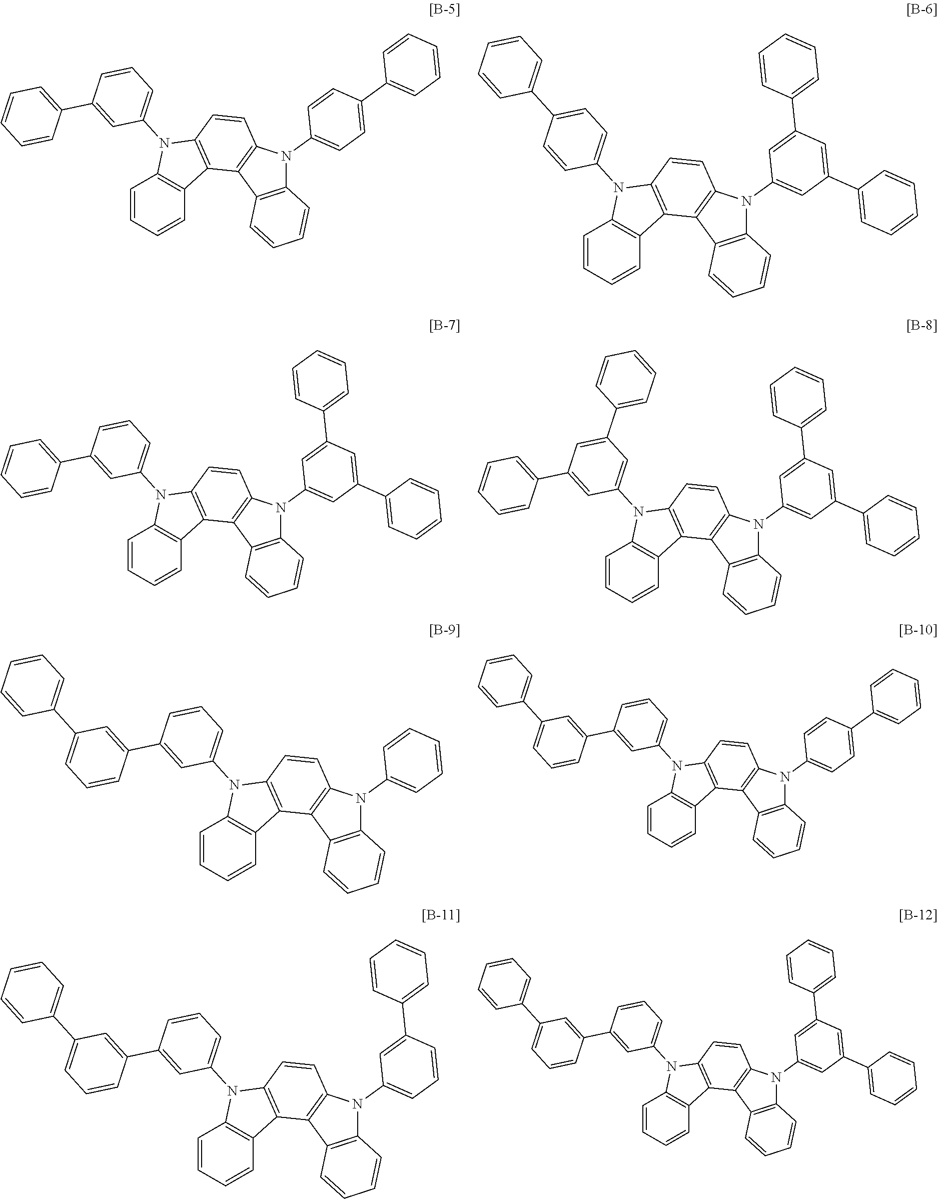

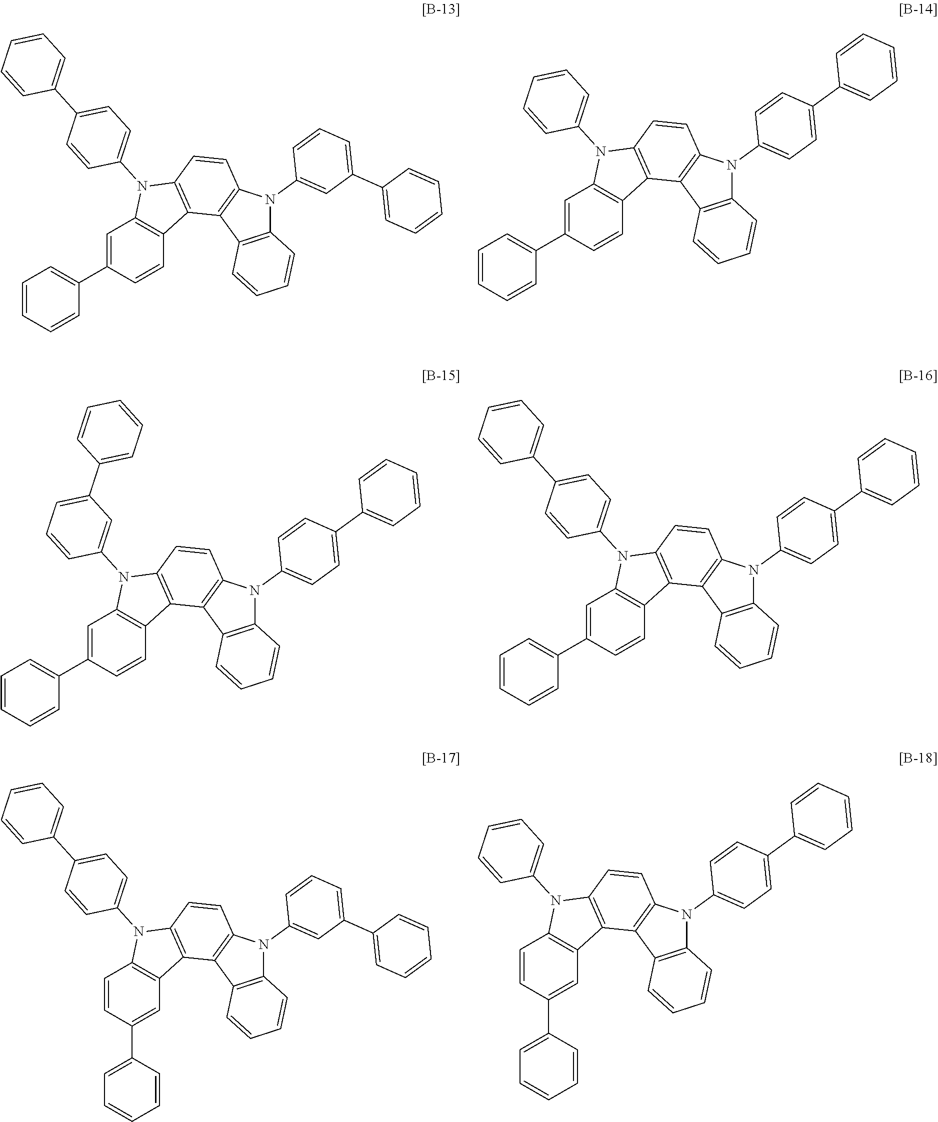

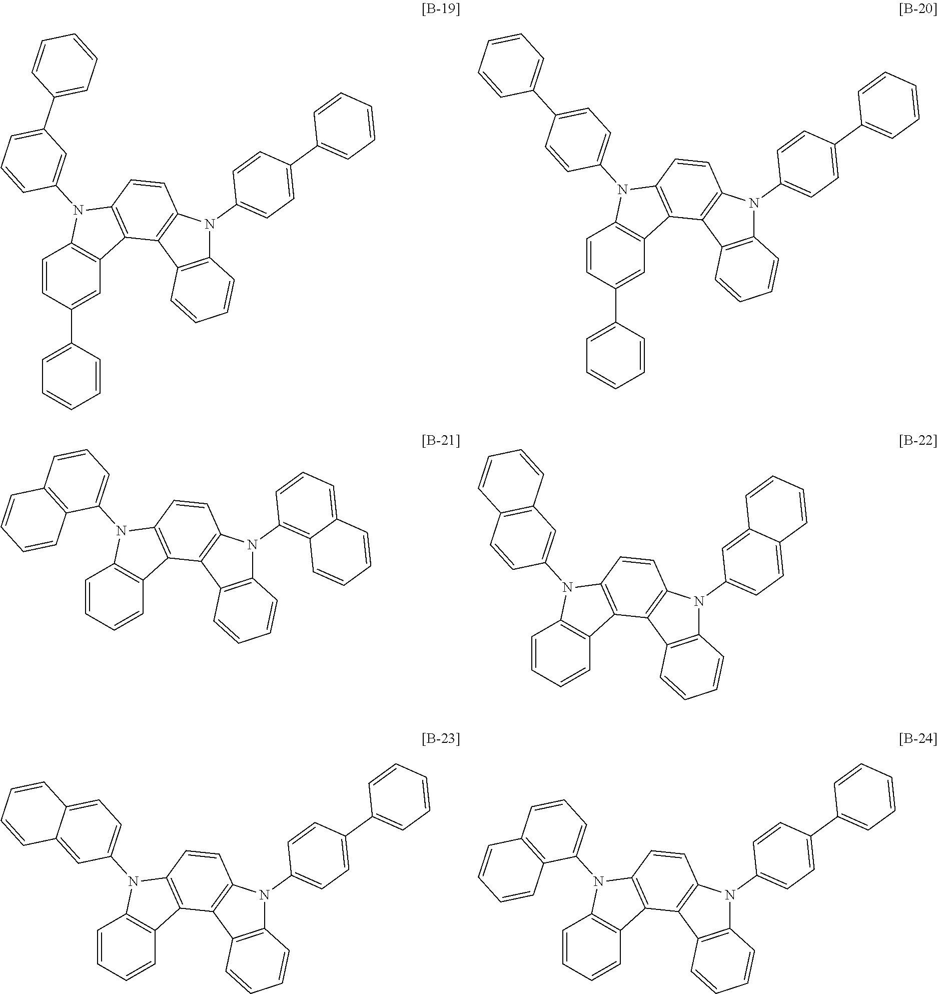

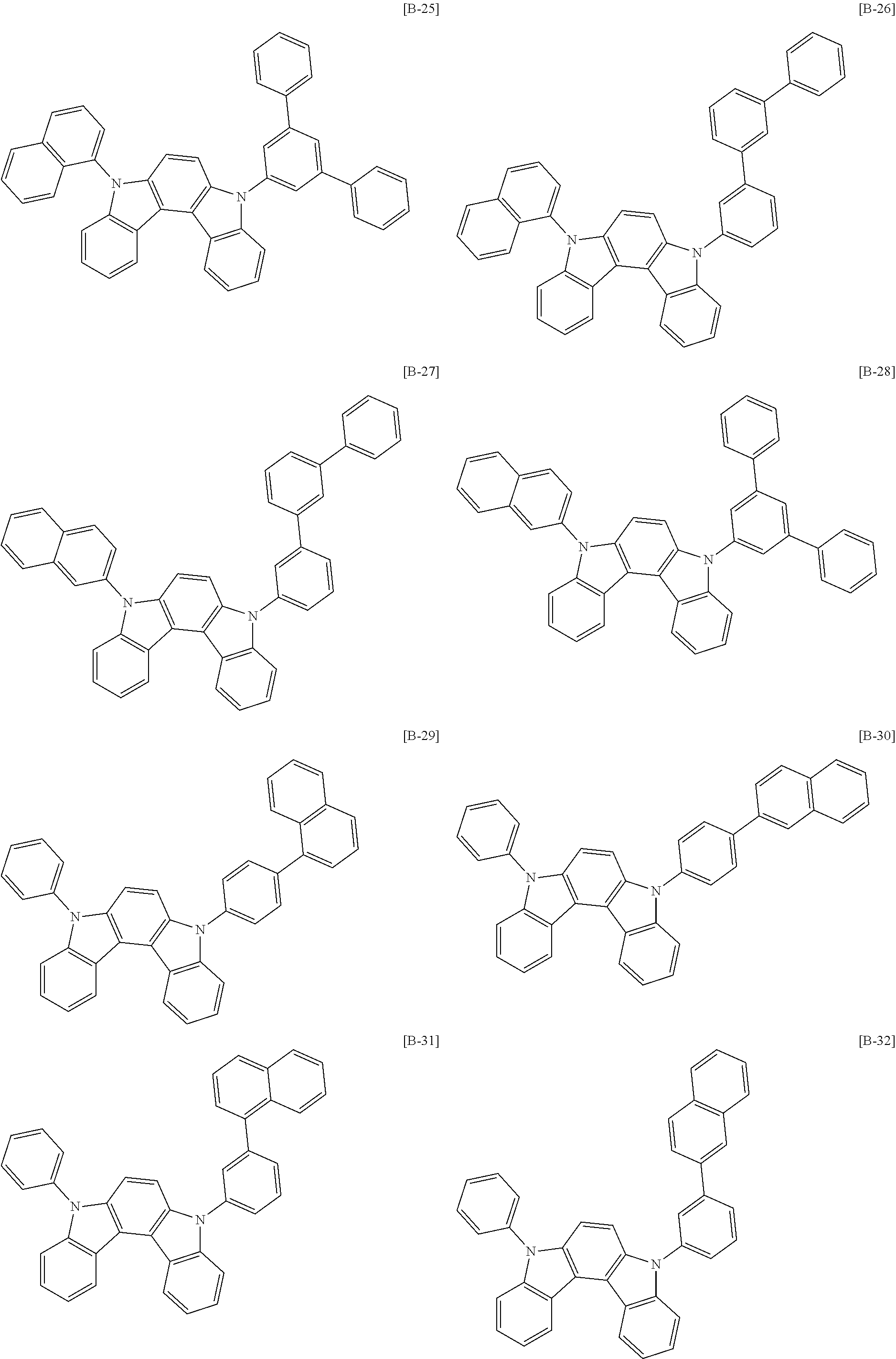

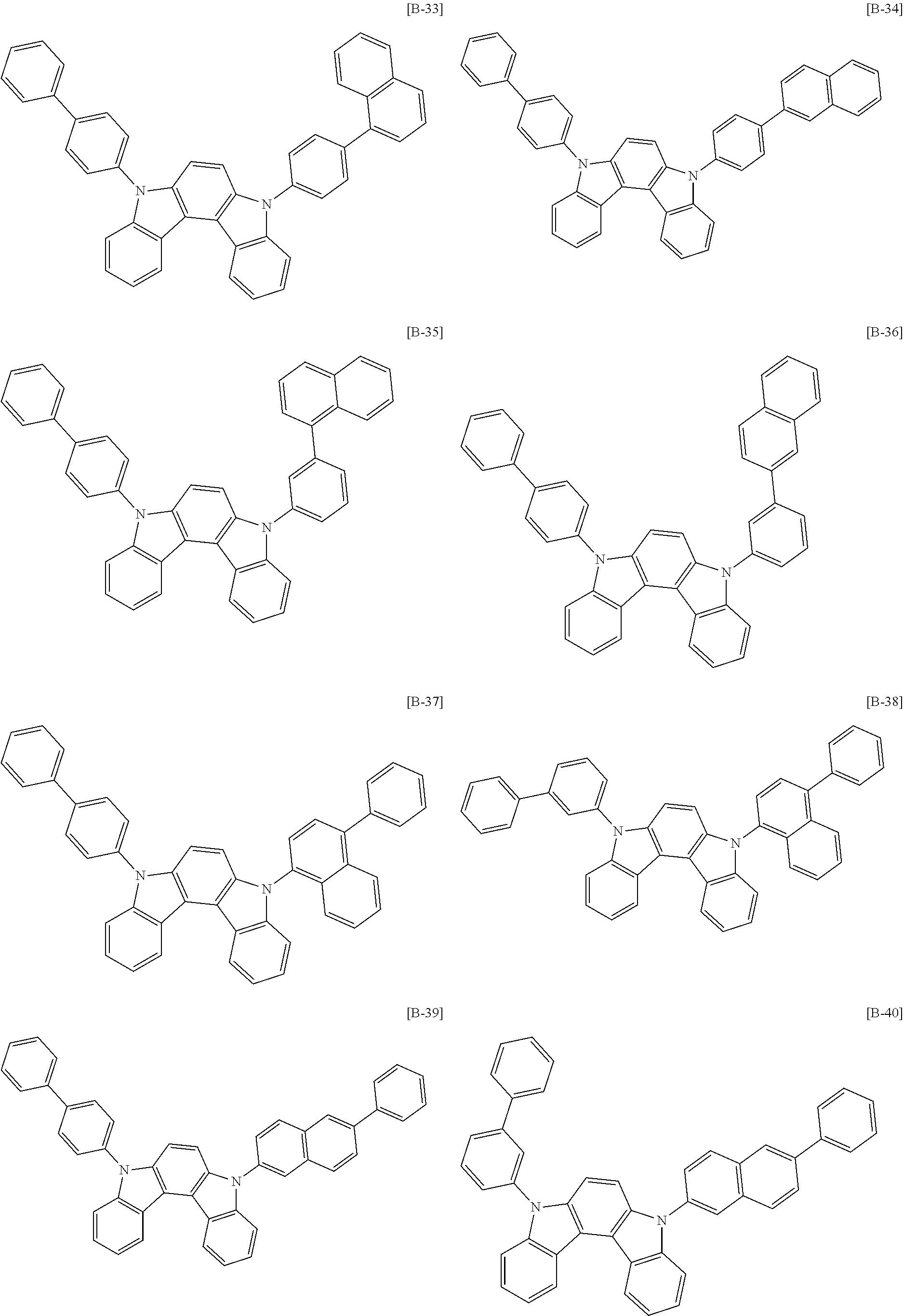

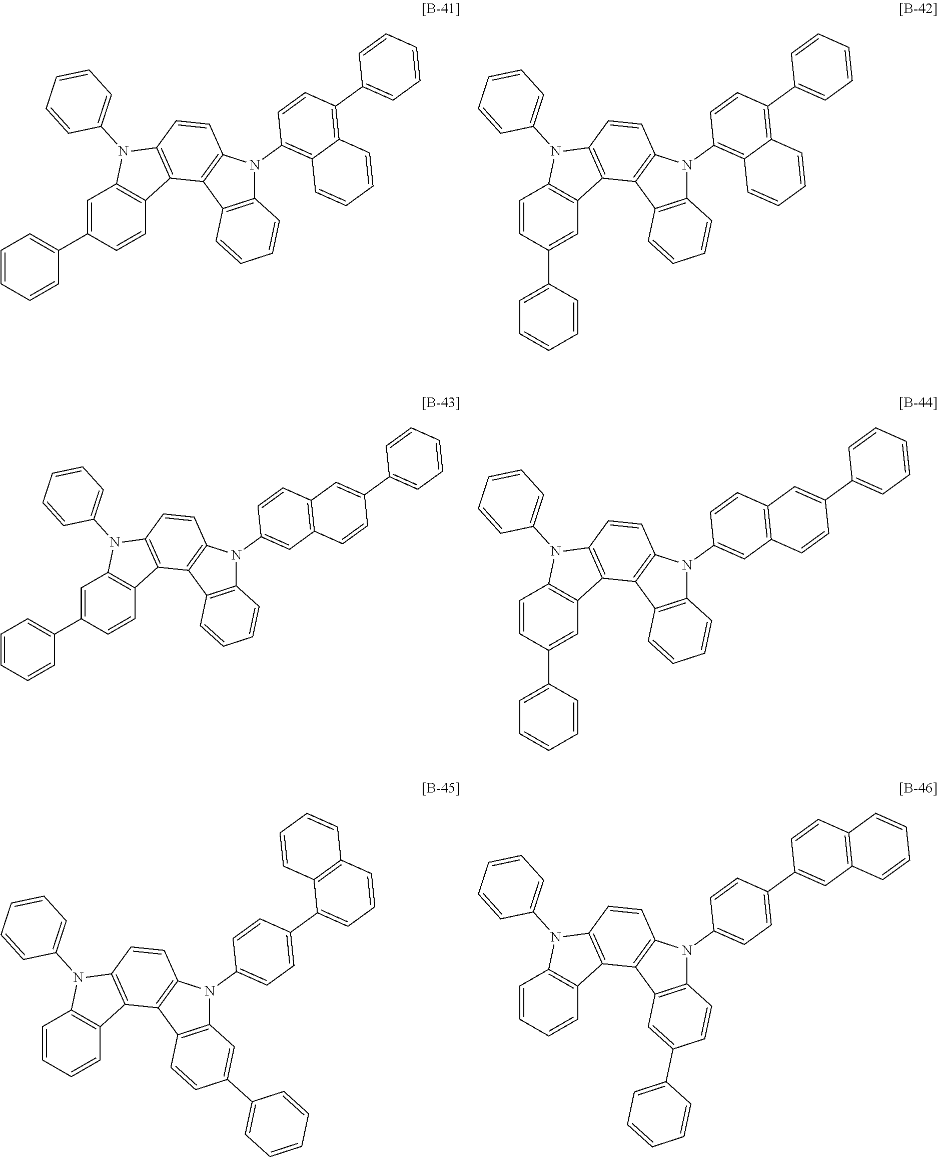

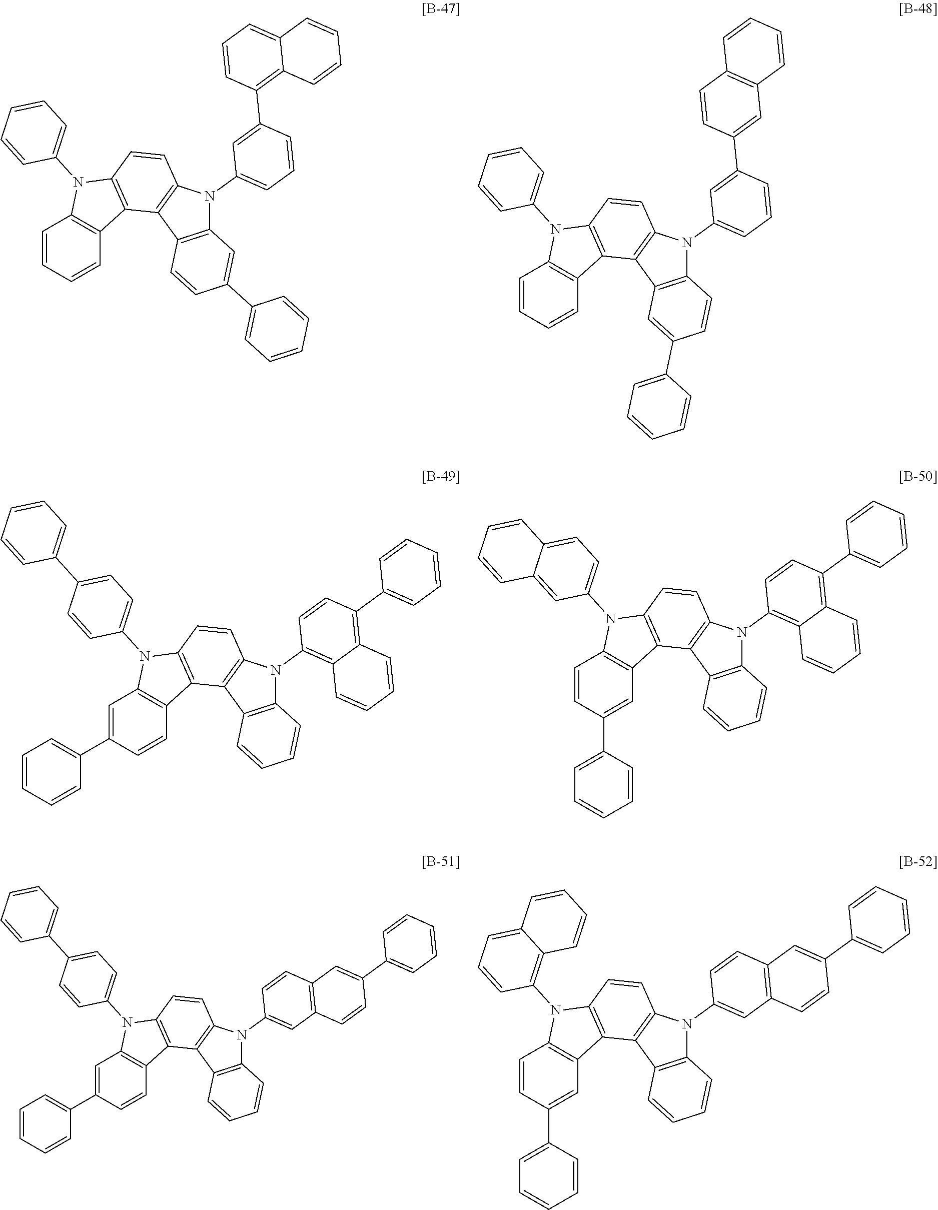

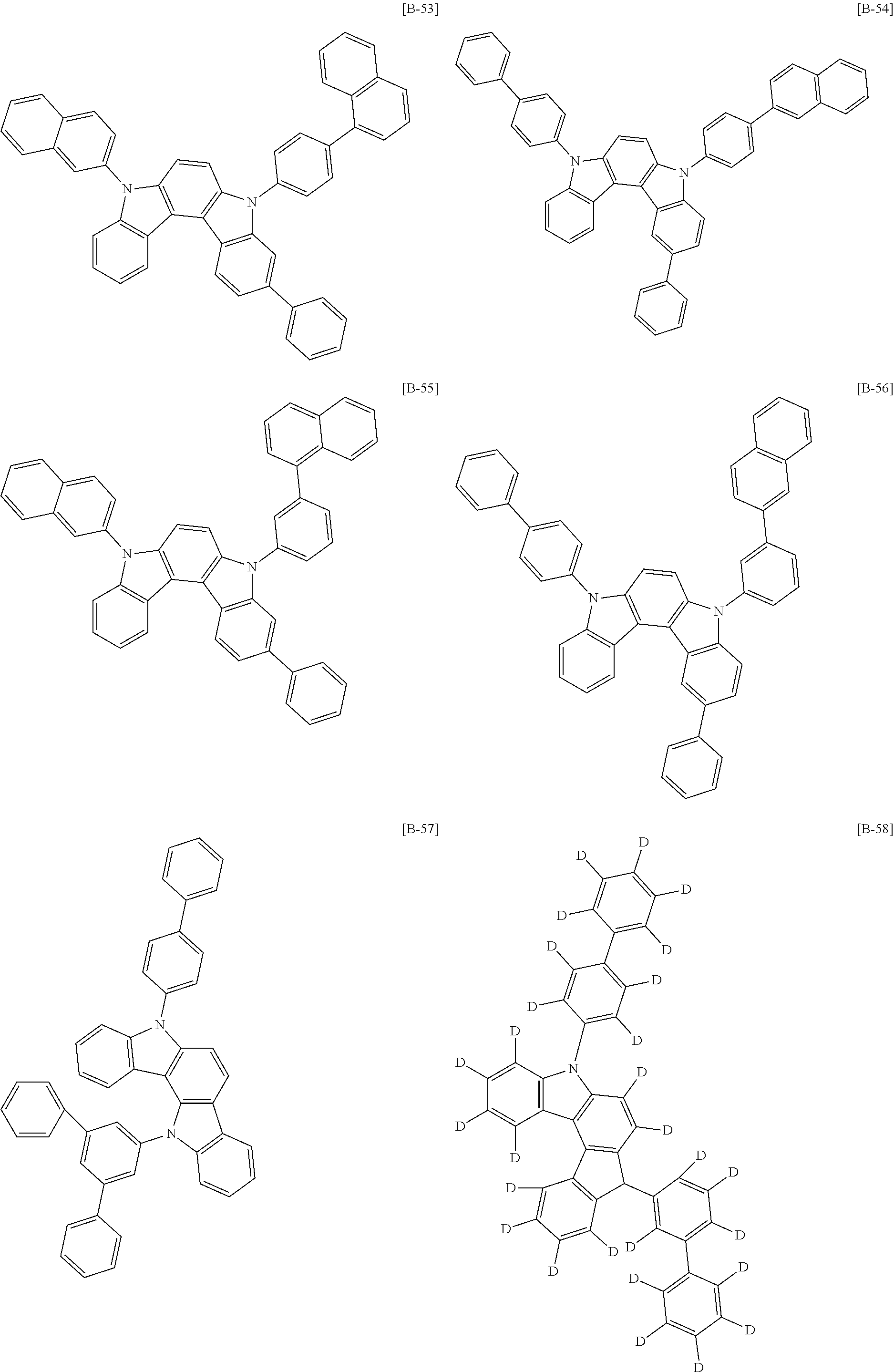

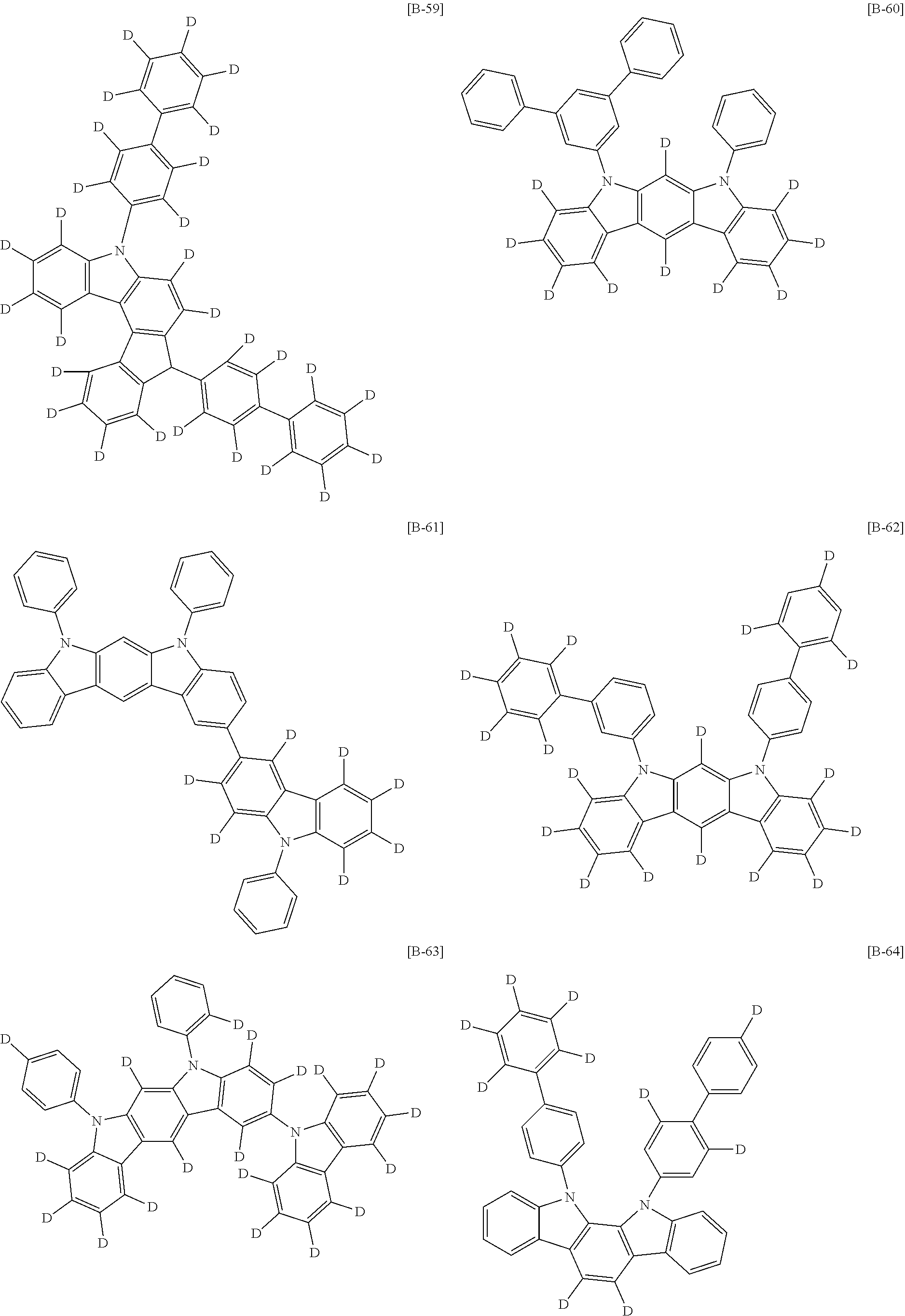

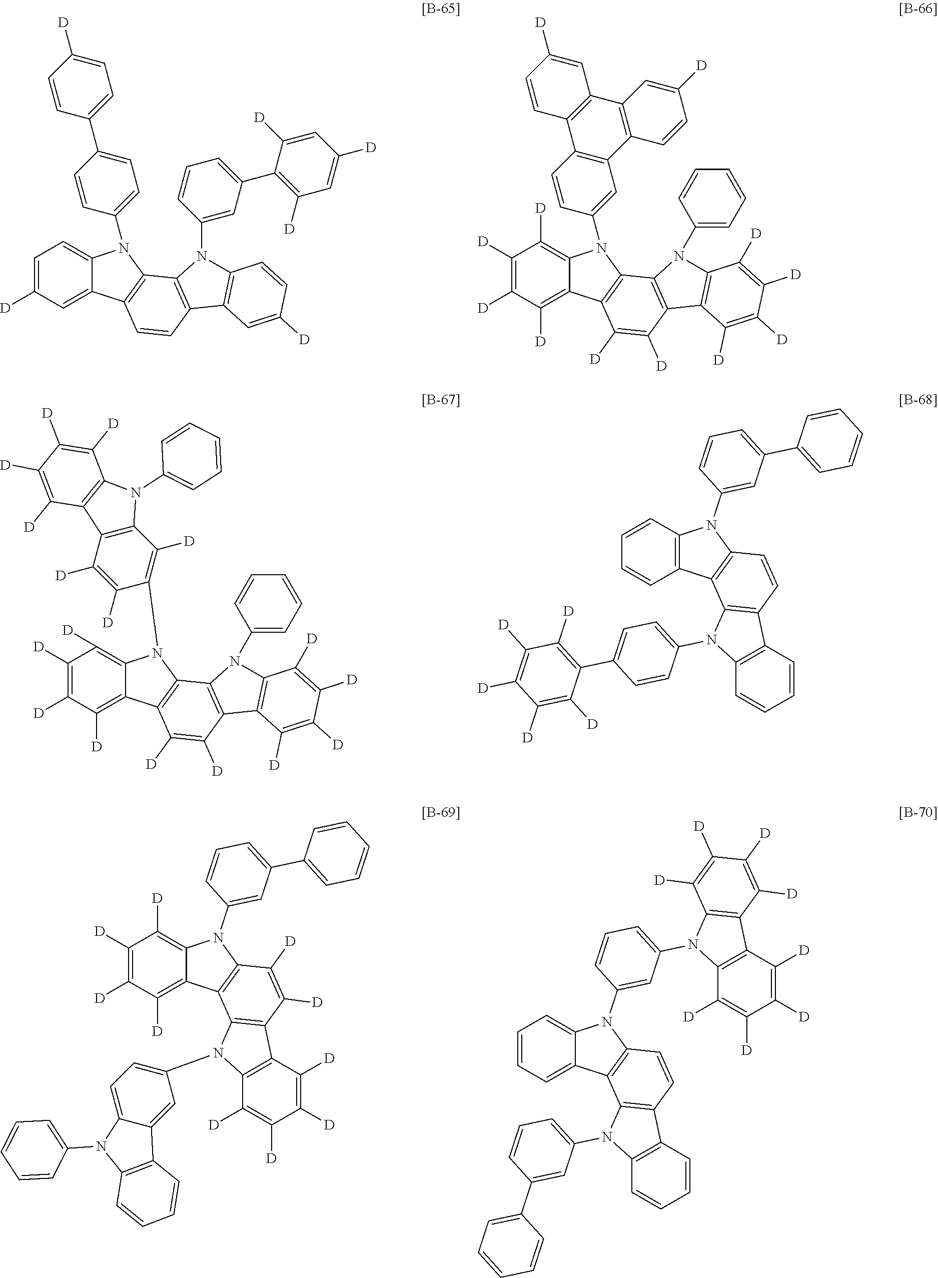

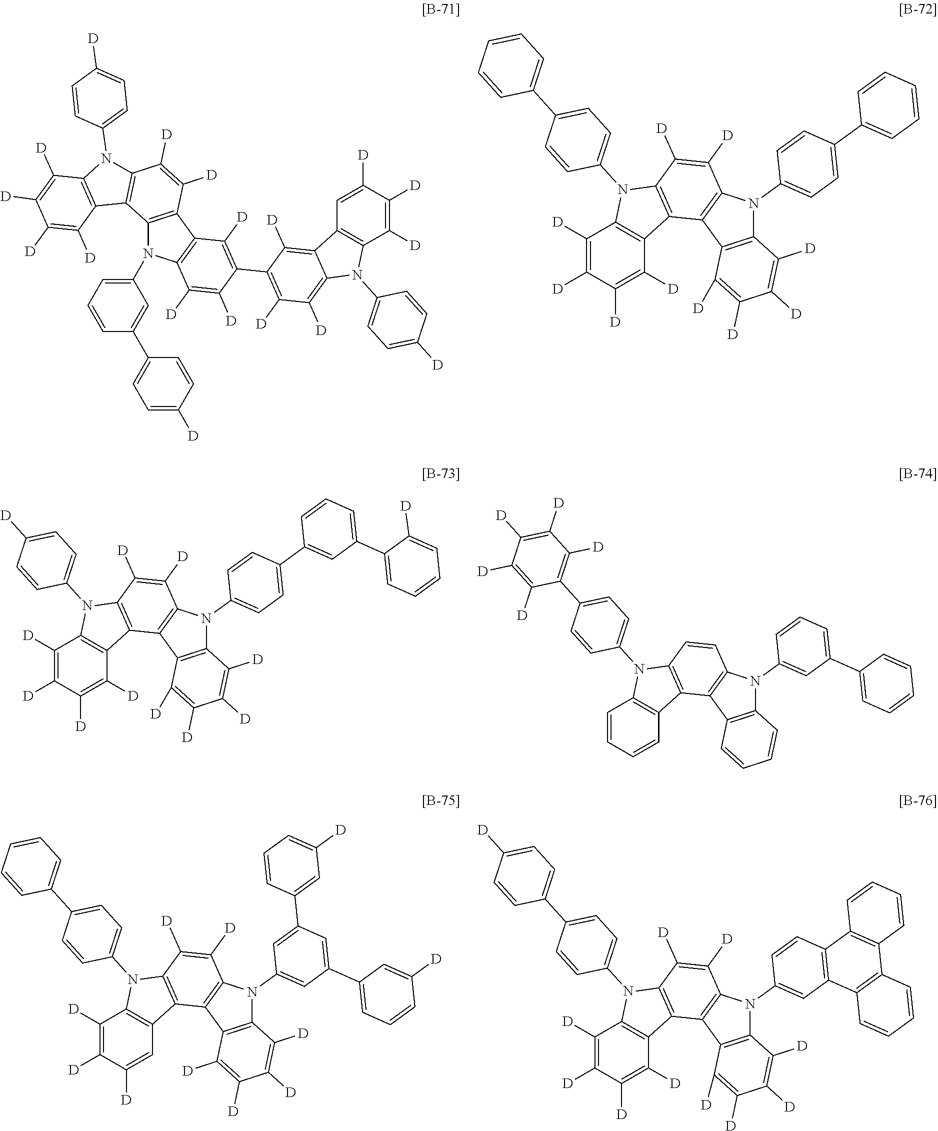

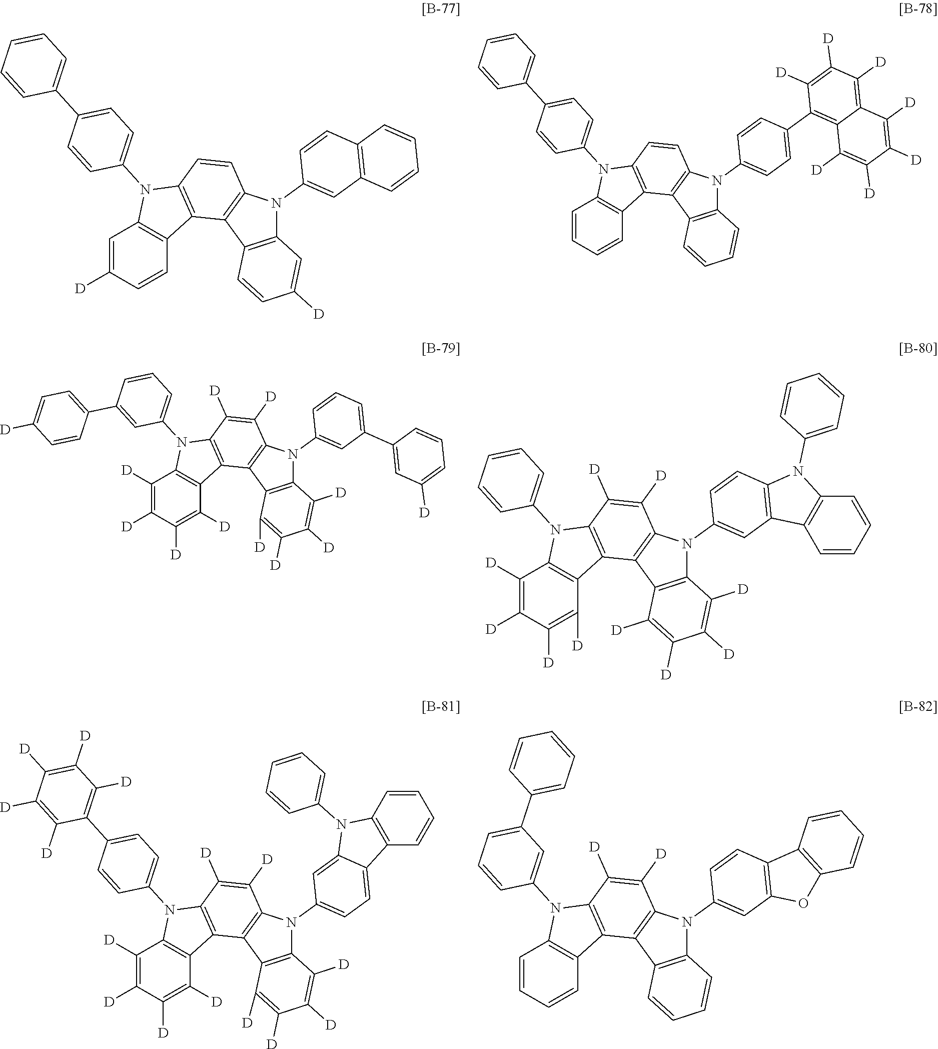

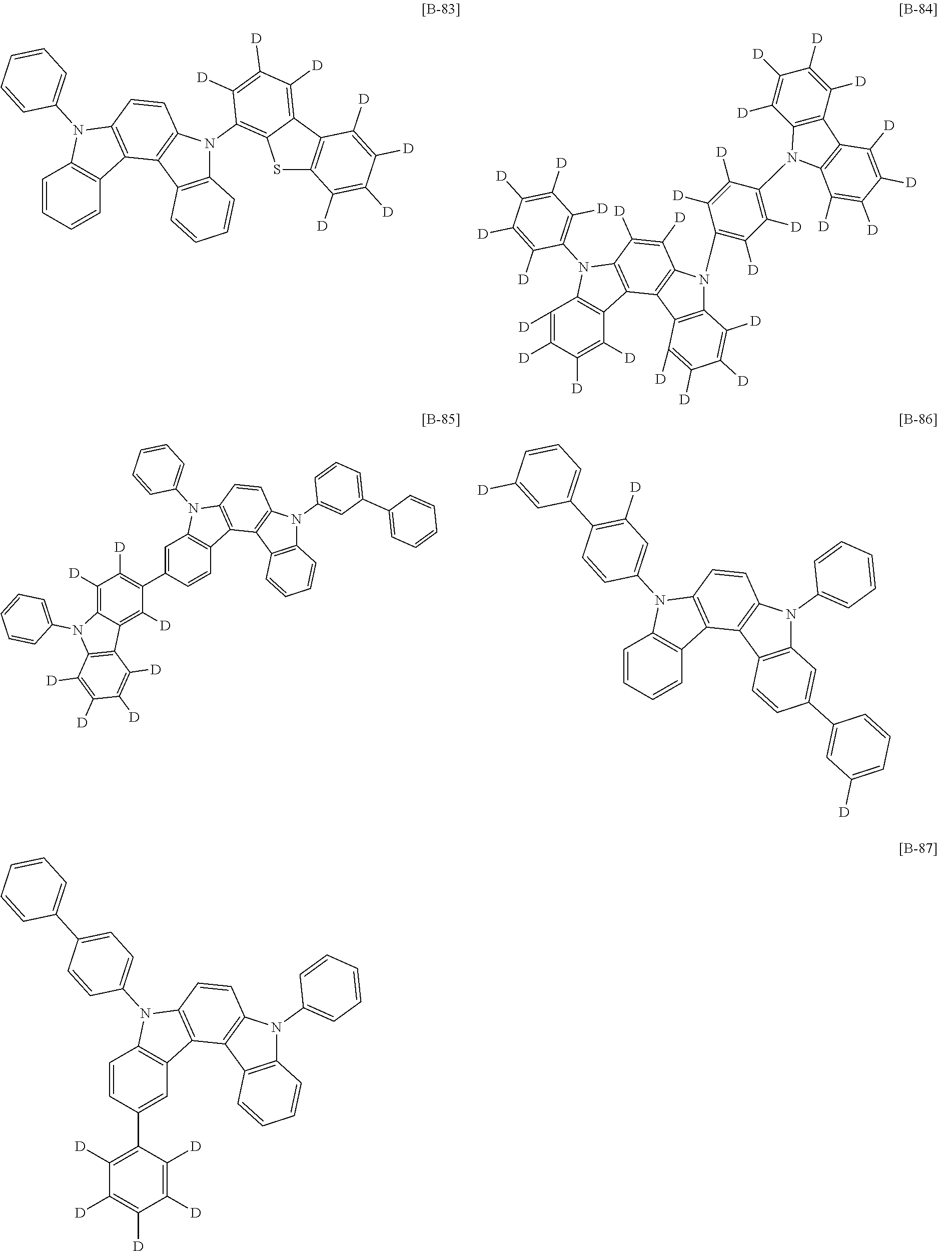

In the composition, the first compound and the second compound may be included, for example, in a weight ratio of about 1:99 to about 99:1.

Within the above range, a desirable weight ratio may be adjusted using an electron transport capability of the first compound and a hole transport capability of the second compound to realize bipolar characteristics and thus to improve efficiency and life-span. Within the range, they may be for example included in a weight ratio of about 10:90 to about 90:10, or about 20:80 to about 80:20, for example a weight ratio of about 20:80 to about 70:30, about 20:80 to about 60:40, and about 20:80 to about 50:50. As a specific example, they may be included in a weight ratio of about 20:80, about 30:70, or about 40:60.

The composition may further include one or more compounds in addition to the aforementioned first and second compounds.

The aforementioned compound for the organic optoelectronic device may be applied in the form of a composition that further includes another, general host material.

The aforementioned compound for the organic optoelectronic device or the composition for the organic optoelectronic device may be implemented as a composition that further includes a dopant. The dopant may be, for example, a phosphorescent dopant, such as a red, green, or blue phosphorescent dopant, and may be, for example, a red or green phosphorescent dopant.

A dopant is a material mixed with the compound or composition for the organic optoelectronic device in a small amount to cause light emission and may be generally a material such as a metal complex that emits light by multiple excitation into a triplet or more. The dopant may be, for example an inorganic, organic, or organic-inorganic compound, and one or more types thereof may be used.

An example of the dopant may be a phosphorescent dopant, and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z.

L⁶MX [Chemical Formula Z]

In an example embodiment of Chemical Formula Z,

M is a metal, and

L⁶ and X are the same as or different from each other, and are ligands forming a complex compound with M.

In Chemical Formula Z, M may be, for example, Ir, Pt, Os, Ti, Zr, Hf, Eu, Tb, Tm, Fe, Co, Ni, Ru, Rh, Pd, or a combination thereof.

In Chemical Formula Z, L⁶ and X may be, for example, a bidentate ligand. Examples of the ligands represented by L⁶ and X may be selected from the chemical formulas of Group A.

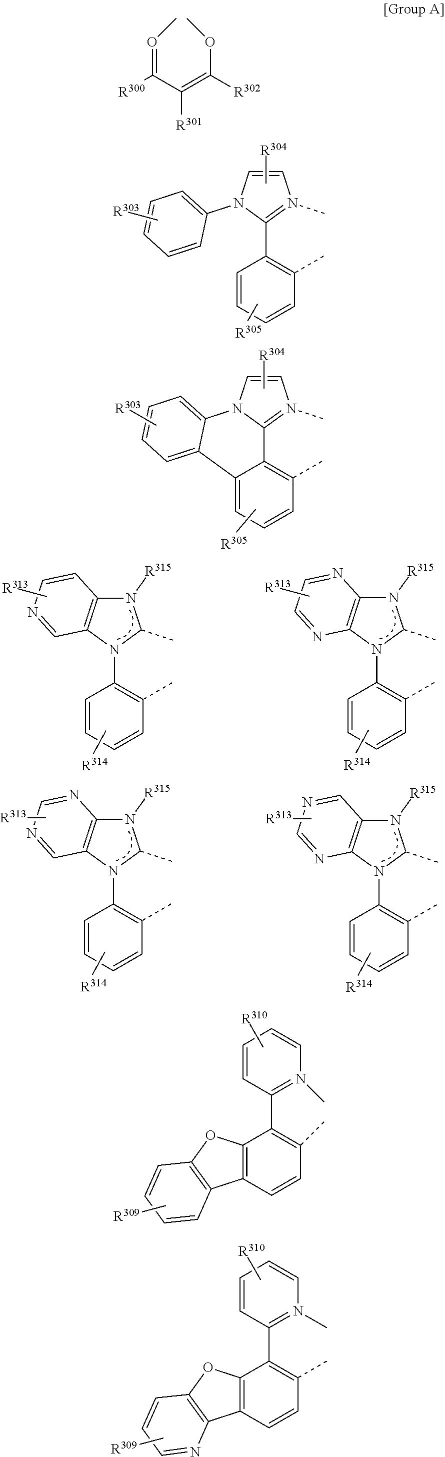

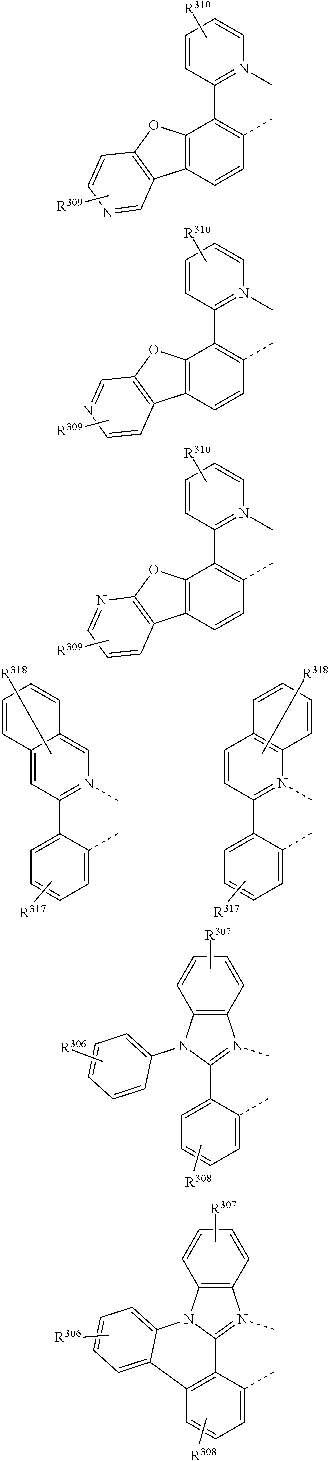

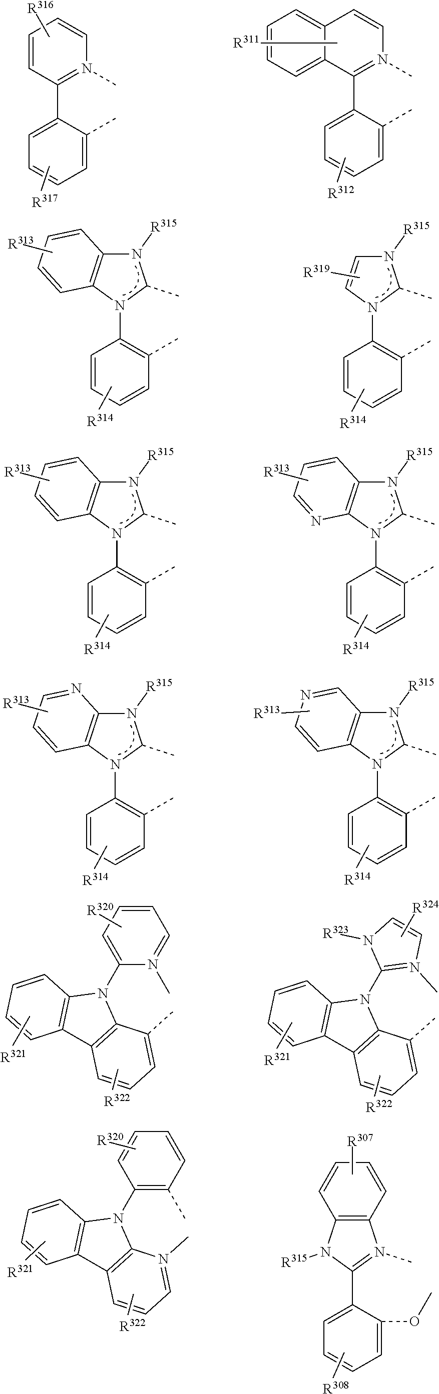

In an example embodiment of Group A,

R³⁰⁰ to R³⁰² are each independently hydrogen, deuterium, a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ are each independently hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

As an example of a composition including a dopant, a dopant represented by Chemical Formula V may be included.

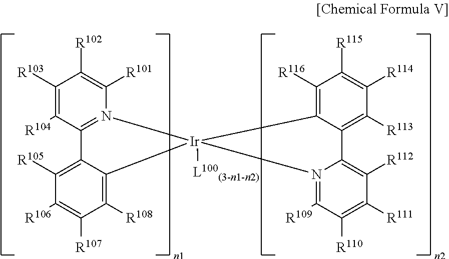

In an example embodiment of Chemical Formula V,

R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ are each independently a C1 to C6 alkyl group,

at least one of R¹⁰¹ to R¹¹⁶ is a functional group represented by Chemical Formula V-1,

L¹⁰⁰ is a bidentate ligand of a monovalent anion, and is a ligand that coordinates to iridium through a lone pair of electrons of carbon or heteroatom,

n1 and n2 are each independently any one of integers of 0 to 3, and

n1+n2 is any one of integers of 1 to 3,

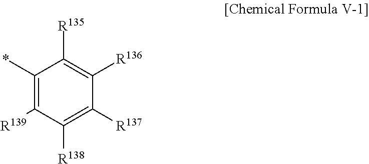

wherein, in Chemical Formula V-1,

R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴ and

* means a portion linked to a carbon atom.

As an example of the composition including a dopant, a dopant represented by Chemical Formula Z-1 may be included.

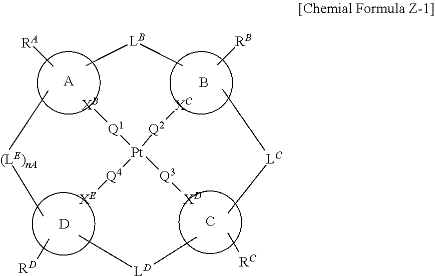

In an example embodiment of Chemical Formula Z-1,

rings A, B, C, and D may each independently be a 5-membered or 6-membered carbocyclic or heterocyclic ring;

R^{A}, R^{B}, R^{C}, and R^{D} may each independently be mono-, di-, tri-, or tetra-substituted, or unsubstituted;

L^{B}, L^{C}, and L^{D} may each independently be selected from a direct bond, BR, NR, PR, O, S, Se, C═O, S═O, SO₂, CRR′, SiRR′, GeRR′, and a combination thereof,

when nA is 1, L^{E} may be selected from a direct bond, BR, NR, PR, O, S, Se, C═O, S═O, SO₂, CRR′, SiRR′, GeRR′, and a combination thereof, when nA is 0, L^{E} may not exist; and

R^{A}, R^{B}, R^{C}, R^{D}, R, and R′ may each independently be selected from hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof, any adjacent R^{A}, R^{B}, R^{C}, R^{D}, R, and R′ are optionally linked to each other to provide a ring; X^{B}, X^{C}, X^{D}, and X^{E} may each independently be selected from carbon and nitrogen; and Q¹, Q², Q³, and Q⁴ may each independently be oxygen or a direct bond.

The dopant according to an example embodiment may be a platinum complex, and may be, for example, represented by Chemical Formula VI.

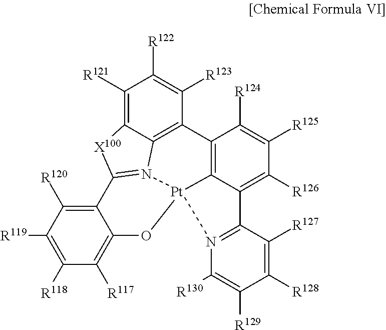

In an example embodiment of Chemical Formula VI,

X¹⁰⁰ is selected from O, S, and NR¹³¹,

R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ are each independently a C1 to C6 alkyl group, and at least one of R¹¹⁷ to R¹³¹ is —SiR¹³²R¹³³R¹³⁴ or a tert-butyl group.

Hereinafter, an organic optoelectronic device including the aforementioned compound for the organic optoelectronic device or the aforementioned composition for the organic optoelectronic device is described.

The organic optoelectronic device may be any device to convert electrical energy into photoenergy and vice versa without particular limitation, and may be, for example an organic photoelectric device, an organic light emitting diode, an organic solar cell, and an organic photo-conductor drum.

Herein, an organic light emitting diode as one example of an organic optoelectronic device is described referring to drawing.

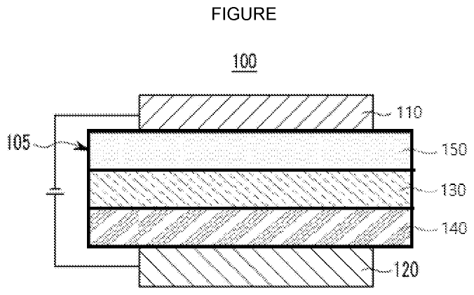

The FIG. 1s a cross-sectional view illustrating an organic light emitting diode according to an example embodiment.

Referring to the FIGURE, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, and polyaniline.

The cathode 110 may be made of a conductor having a small work function to help electron injection, and may be for example a metal, a metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof, or a multi-layer structure material such as LiF/Al, LiO₂/Al, LiF/Ca, and BaF₂/Ca.

The organic layer 105 may include the aforementioned compound for the organic optoelectronic device or composition for the organic optoelectronic device.

The organic layer 105 may include a light emitting layer 130, and the light emitting layer 130 may include the aforementioned compound for the organic optoelectronic device or composition for the organic optoelectronic device.

The composition for the organic optoelectronic device further including the dopant may be, for example, a green light emitting composition.

The light emitting layer 130 may include, for example, the aforementioned compound for the organic optoelectronic device or composition for the organic optoelectronic device as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons. Specifically, the hole transport region 140 may include a hole transport layer between the anode 120 and the light emitting layer 130, and a hole transport auxiliary layer between the light emitting layer 130 and the hole transport layer and at least one of the compounds of Group B may be included in at least one of the hole transport layer and the hole transport auxiliary layer.

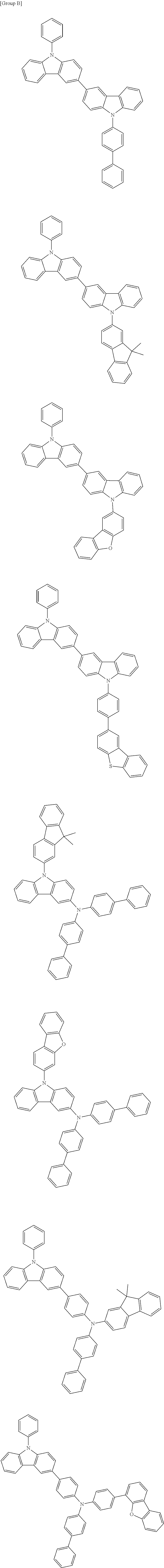

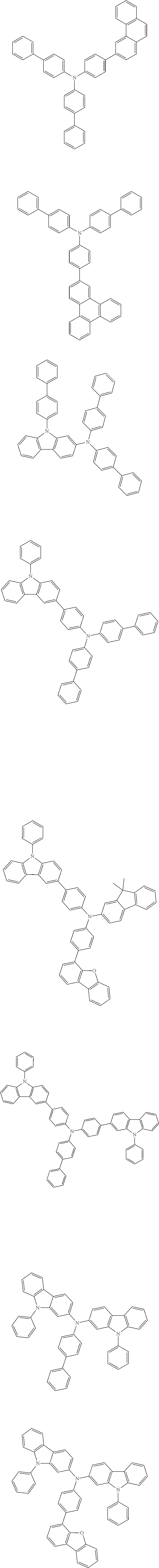

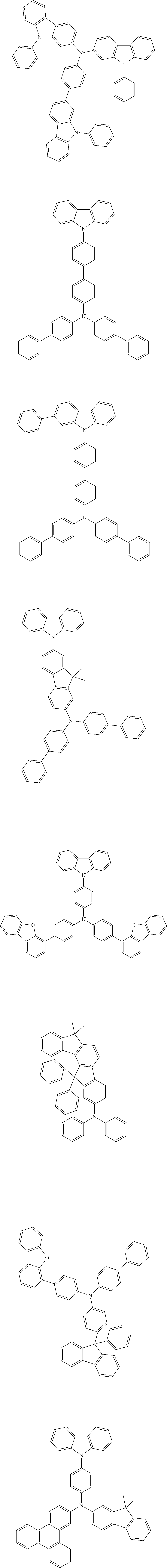

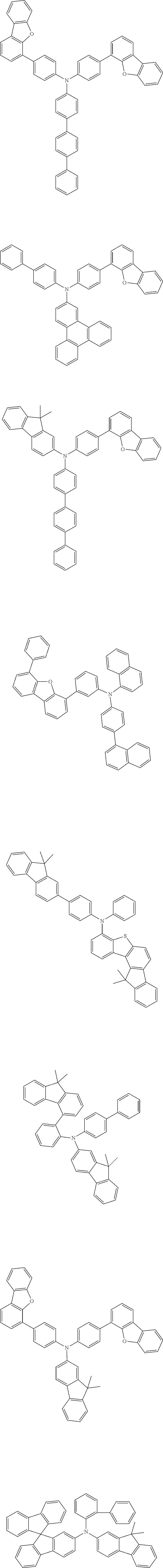

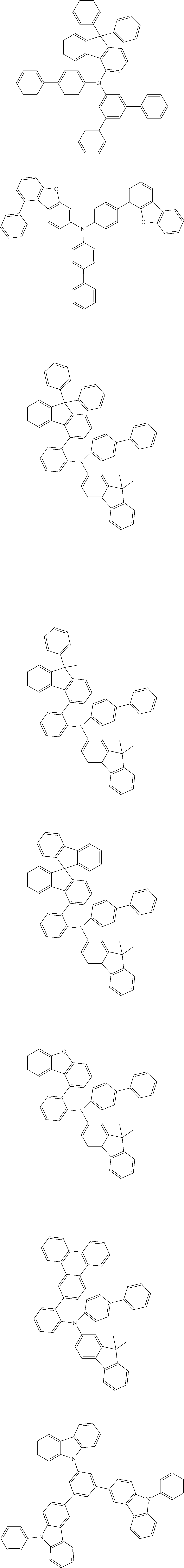

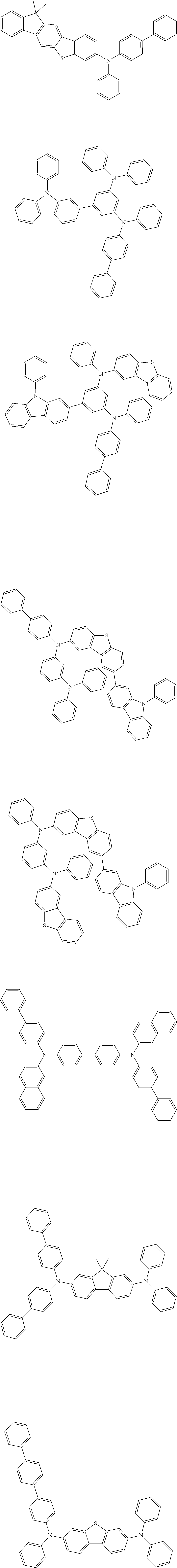

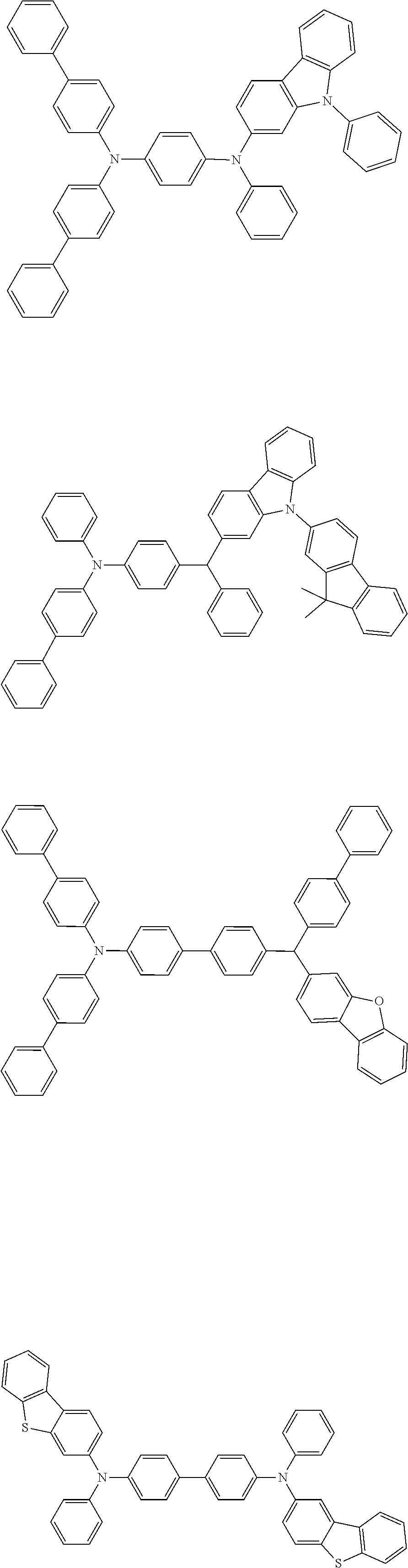 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(C(c5ccccc5)c5ccc(N(c6ccccc6)c6ccc(-c7ccccc7)cc6)cc5)cc43)cc21 ; c1ccc(-c2ccc(C(c3ccc(-c4ccc(N(c5ccc(-c6ccccc6)cc5)c5ccc(-c6ccccc6)cc5)cc4)cc3)c3ccc4c(c3)oc3ccccc34)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

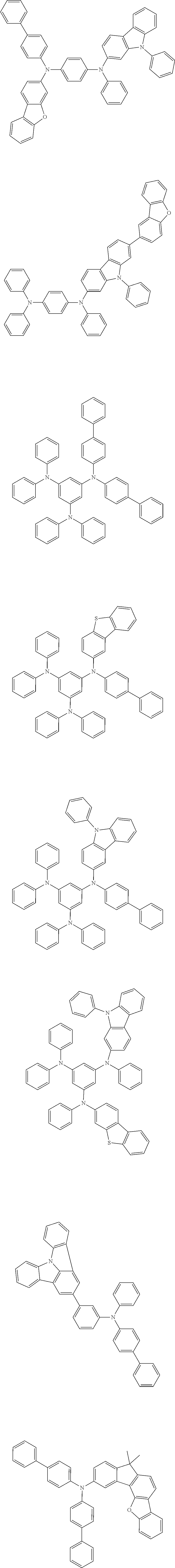

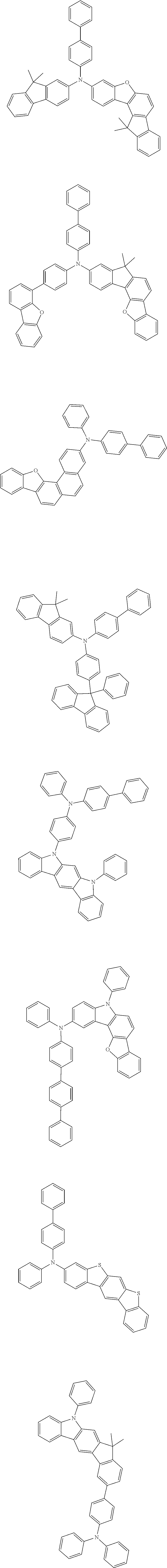

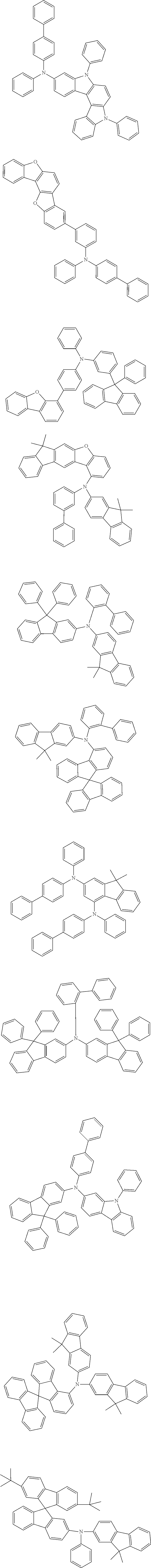

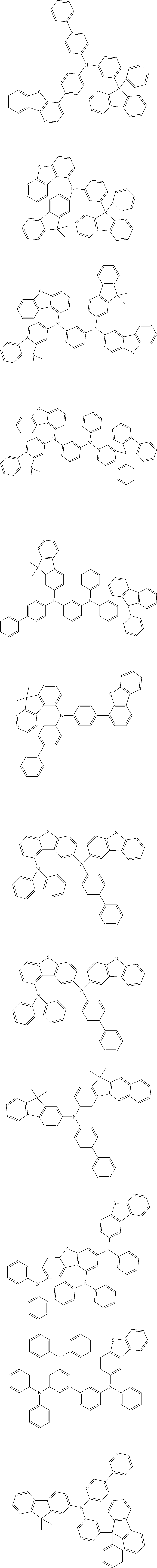

In the hole transport region 140, compounds disclosed in U.S. Pat. No. 5,061,569, JP 1993-009471 A, WO 1995-009147 A1, JP 1995-126615 A, JP 1998-095973 A, and the like, and compounds similar thereto, may be used in addition to the compound.

In addition, the charge transport region may be, for example, an electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

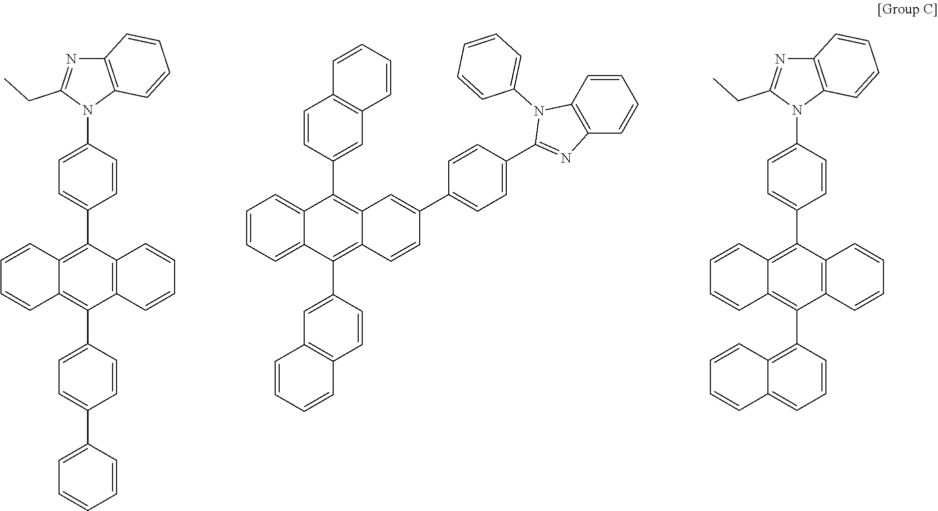 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

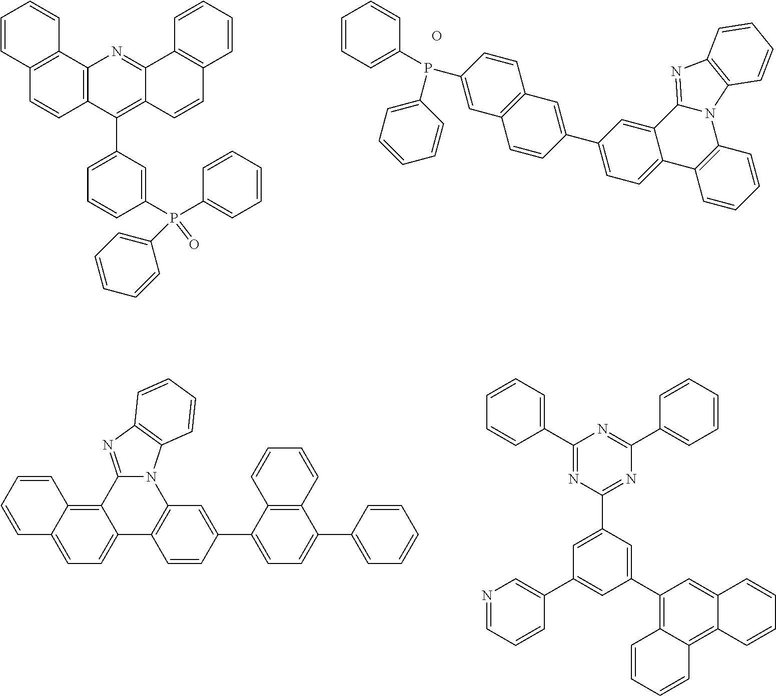

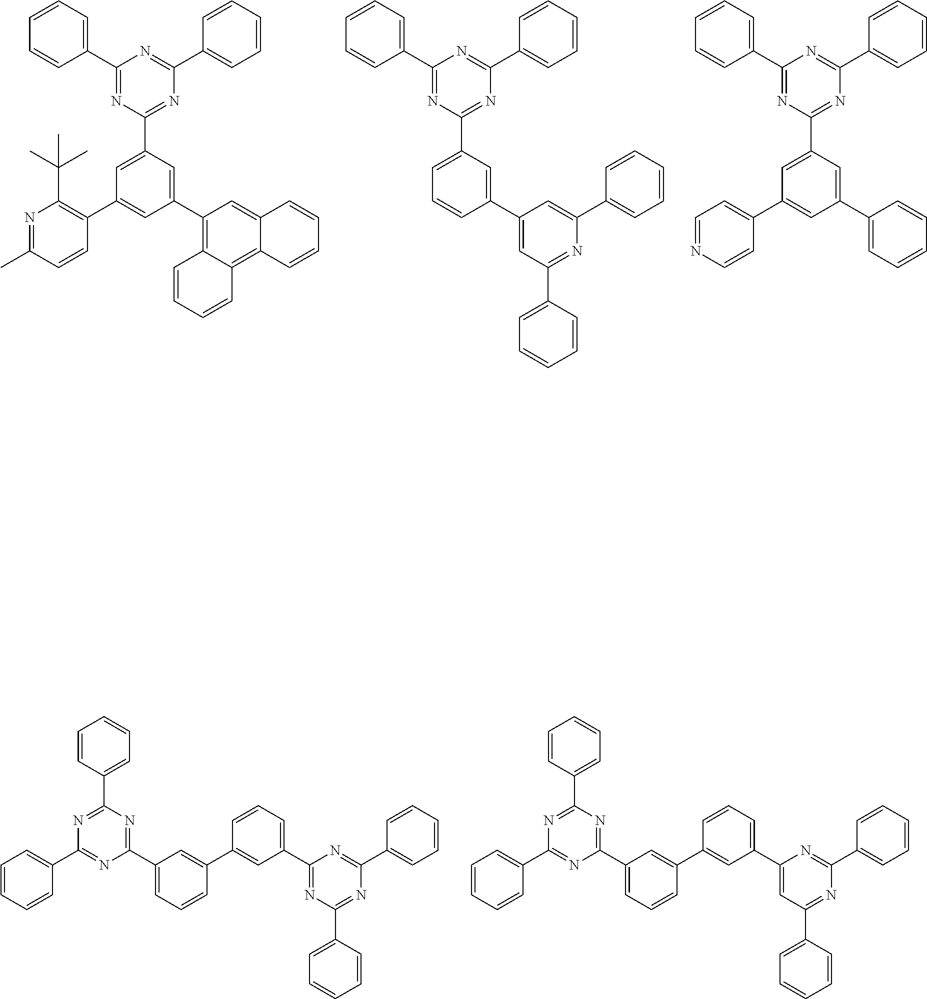

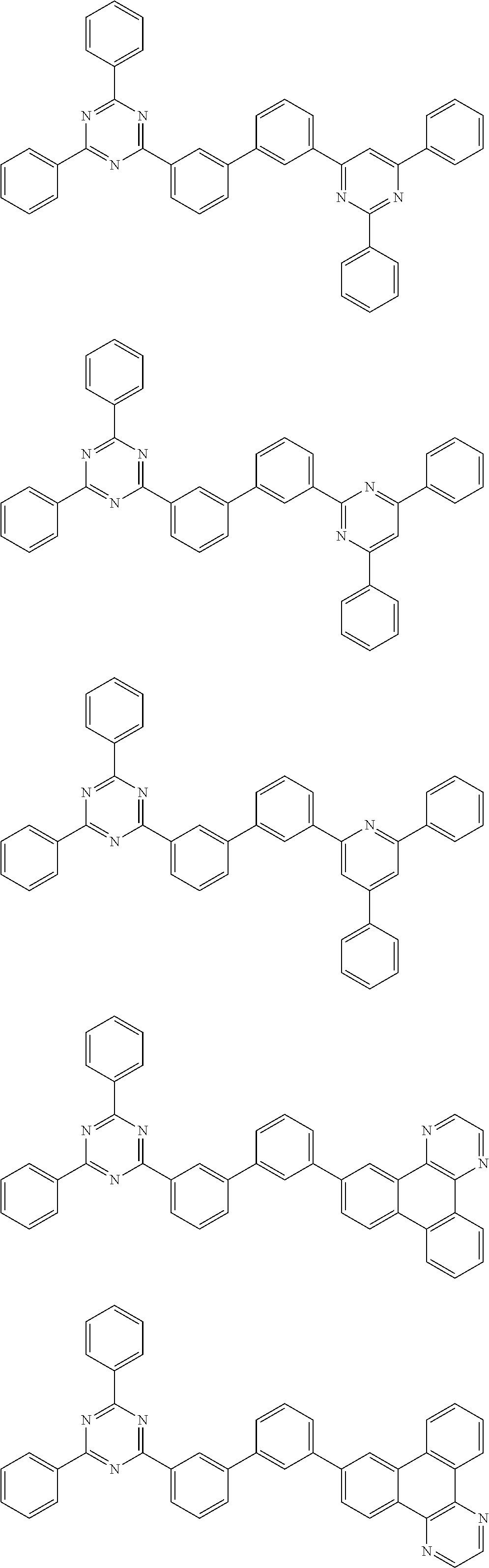 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

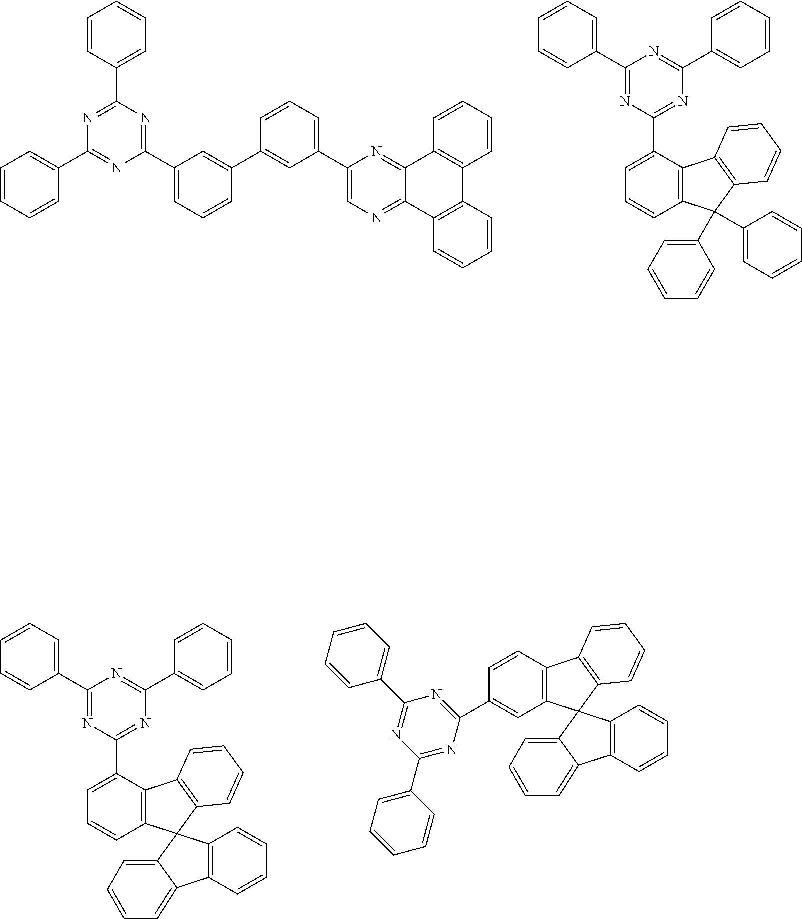 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1]

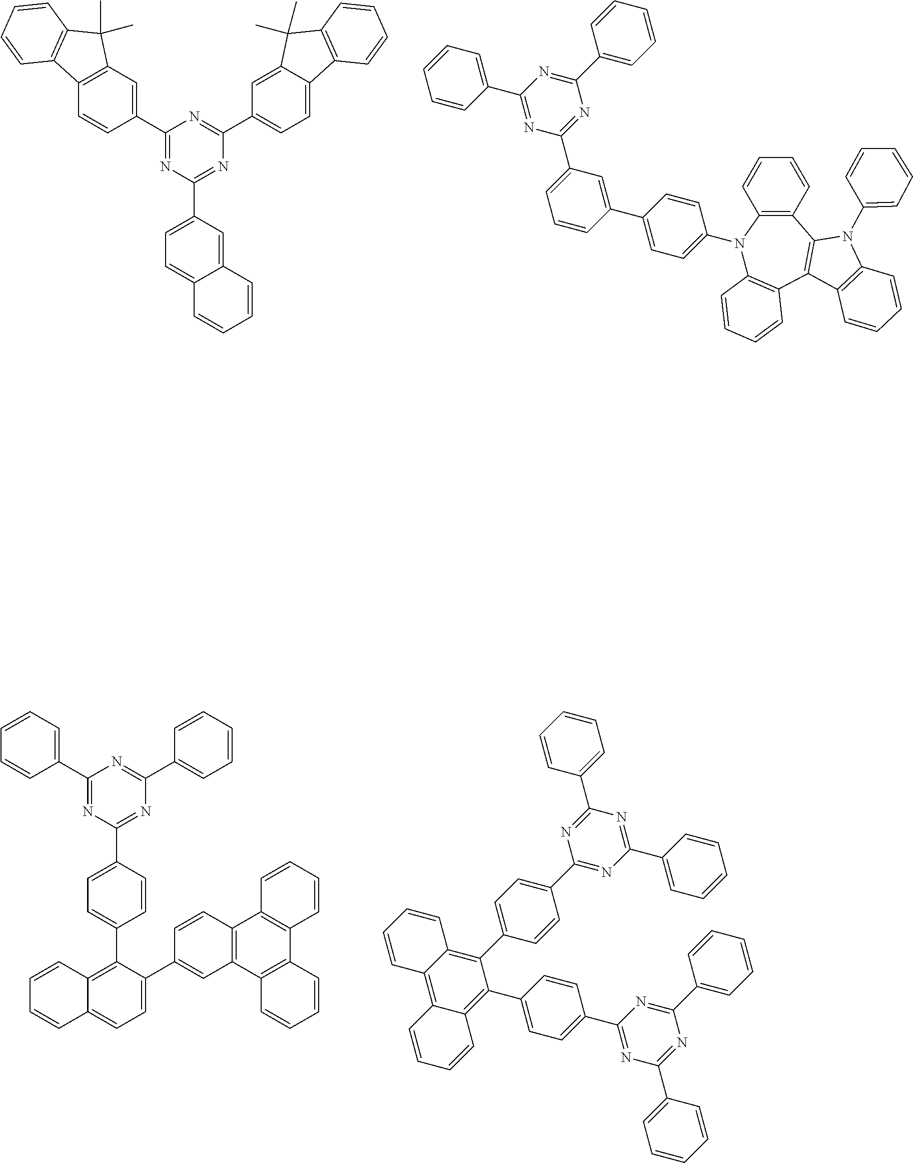 [SMILES: CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1 ; c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1]

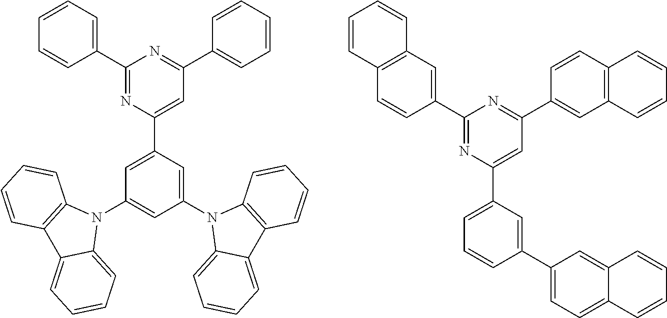 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1cc(-c2ccc3ccccc3c2)cc(-c2cc(-c3ccc4ccccc4c3)nc(-c3ccc4ccccc4c3)n2)c1]

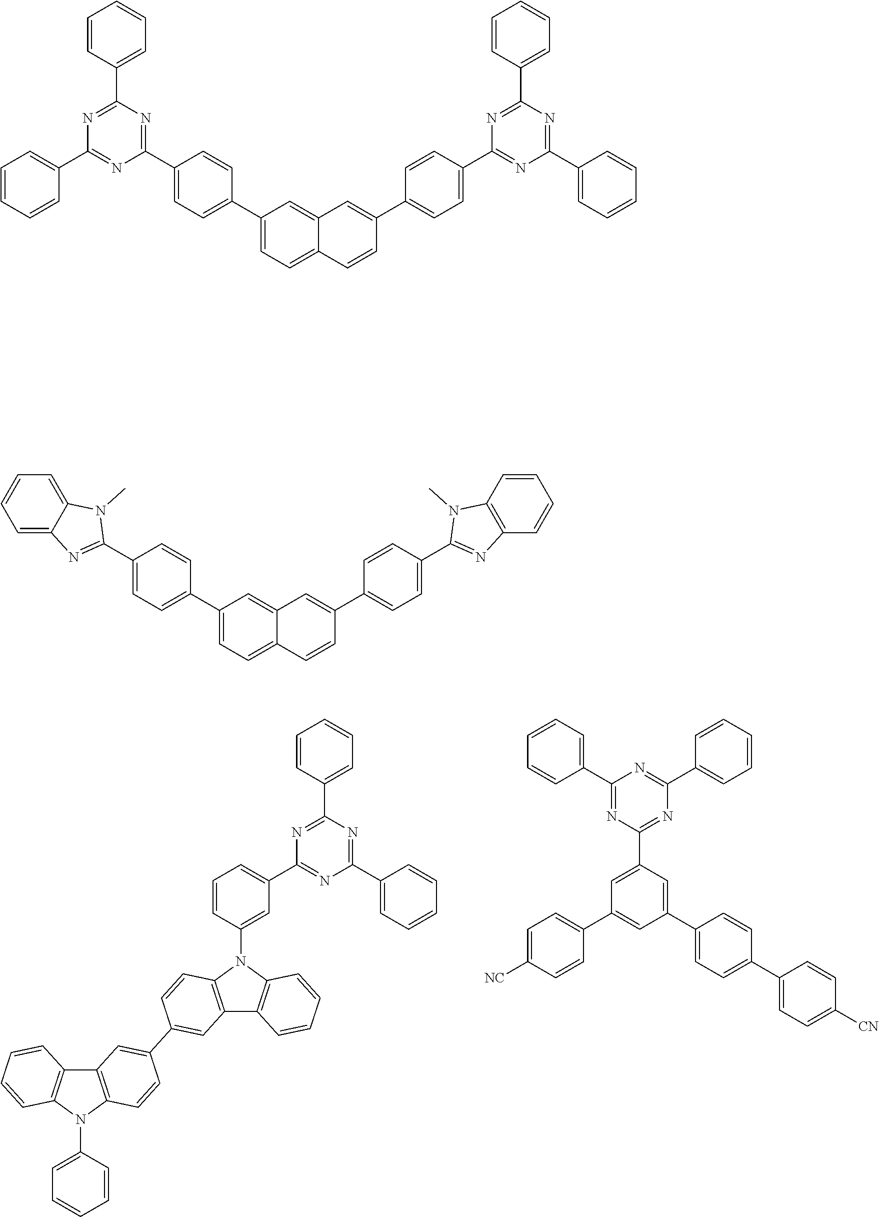 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1]

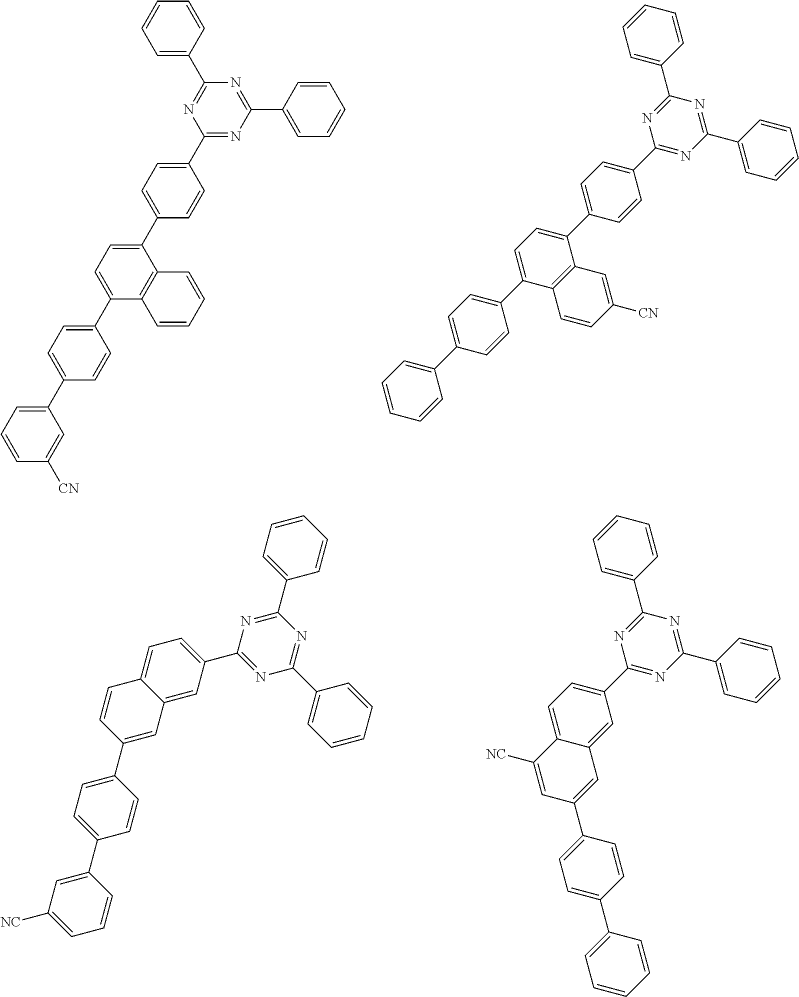 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

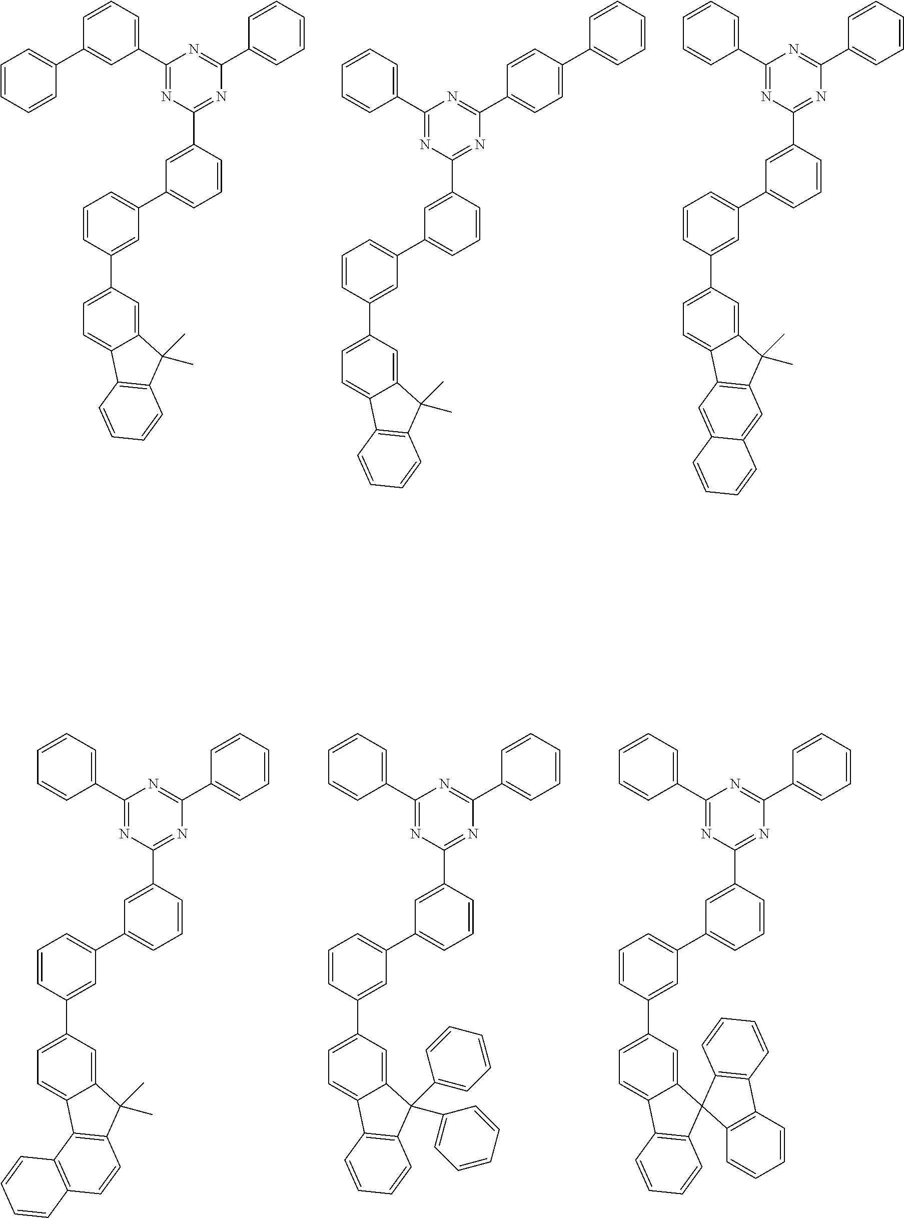

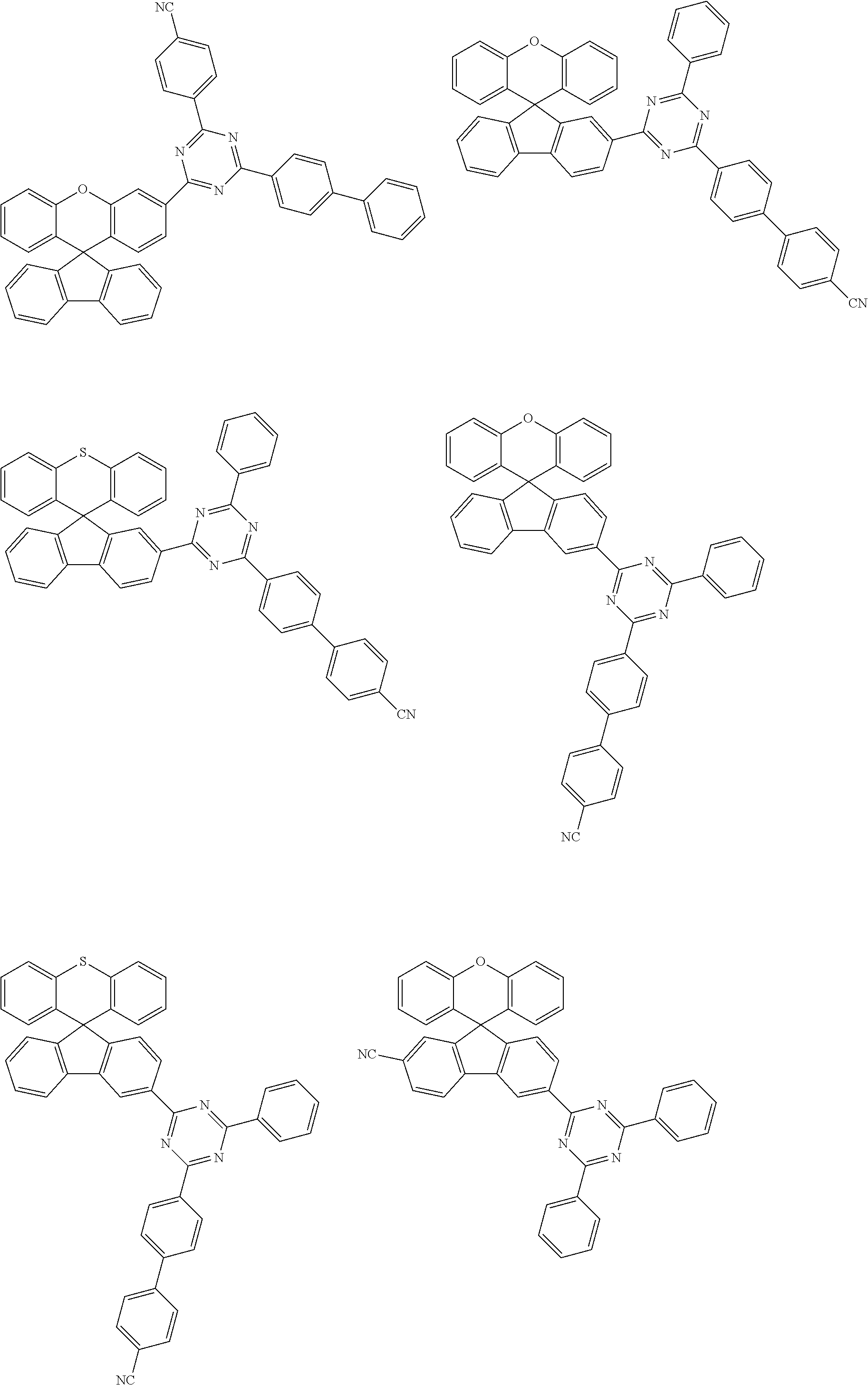

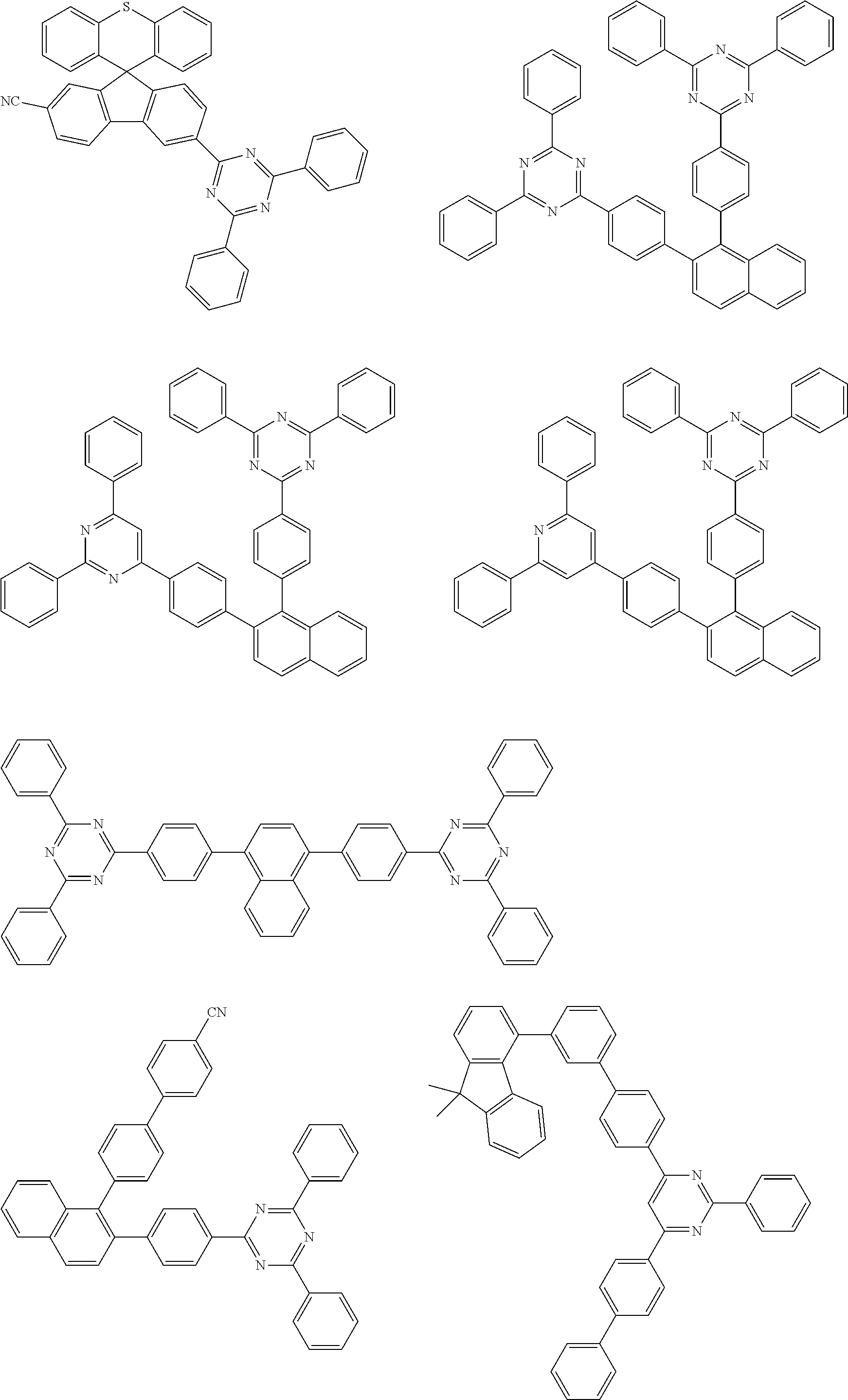

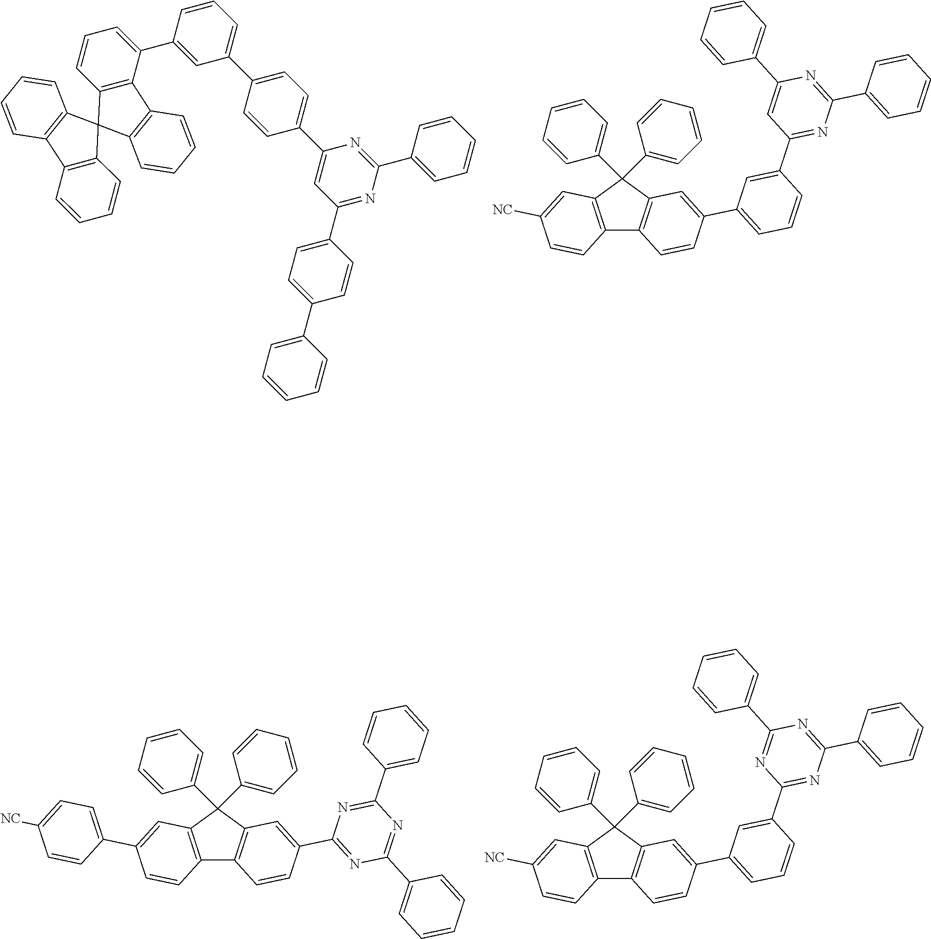

An example embodiment may provide an organic light emitting diode including a light emitting layer as an organic layer.

Another example embodiment may provide an organic light emitting diode including a light emitting layer and a hole transport region as an organic layer.

Another example embodiment may provide an organic light emitting diode including a light emitting layer and an electron transport region as an organic layer.

Referring to FIG. 1, the organic light emitting diode according to an example embodiment may include a hole transport region 140 and an electron transport region 150 in addition to the light emitting layer 130 as the organic layer 105.

The organic light emitting diode according to an example embodiment may further include an electron injection layer (not shown), a hole injection layer (not shown), etc., in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo Chemical Industry, or P&H tech, or were synthesized by a general method, unless stated otherwise.

(Preparation of Compound for Organic Optoelectronic Device)

### Synthesis Example 1: Synthesis of Compound 1-25

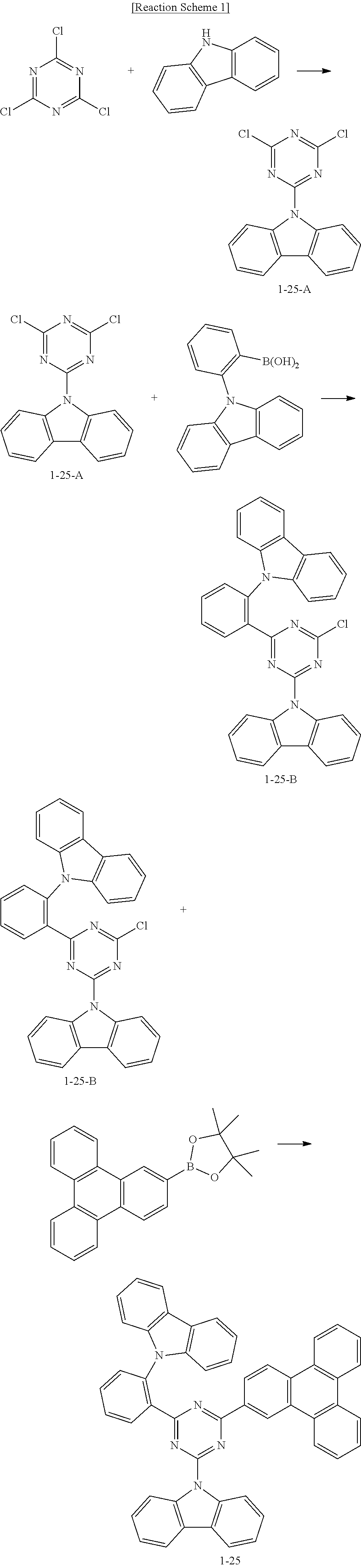

### 1st Step: Synthesis of Intermediate 1-25-A

28.6 g (170.8 mmol) of Carbazole and 180 ml of THF were put in a round flask and then, stirred and cooled to 0° C. in an ice bath, and 68.3 ml (170.8 mmol) of n-BuLi (2.5 M in hexane) was slowly added dropwise thereto. Subsequently, the mixture was additionally stirred for 30 minutes at room temperature. Under a nitrogen flow, 30 g (162.7 mmol) of 2,4,6-trichloro-1,3,5-triazine and 180 ml of THE were put in the flask, and Li reagent prepared in advance was slowly dropwise thereto. The obtained mixture was stirred at room temperature for 1 hour. 300 ml of Distilled water was added thereto, and solids precipitated therefrom were collected through filtration. The solids were dried, obtaining 40.1 g (Yield: 78%) of Intermediate 1-25-A.

### 2nd Step: Synthesis of Intermediate 1-25-B

50 g (158.7 mmol) of Intermediate 1-25-A, 41 g (142.8 mmol) of 2-(9H-carbazol-9-yl)phenylboronic acid (CAS No. 1189047-28-6), 7.8 g (9.5 mmol) of Pd(dppf)Cl₂, 65.8 g (476 mmol) of K₂CO₃, 400 ml of toluene, and 200 ml of distilled water were put in a flask and then, stirred under reflux for 12 hours. When a reaction was completed, the resultant was cooled to room temperature, and solids produced therein were filtered, washed with acetone, and dried, obtaining 61.7 g (Yield: 83%) of Intermediate 1-25-B.

### 3rd Step: Synthesis of Compound 1-25

27.7 g (53.0 mmol) of Intermediate 1-25-B, 19.7 g (55.7 mmol) of (triphenylen-2-yl)boronic acid pinacol ester (CAS No. 890042-13-4), 1.8 g (1.6 mmol) of Pd(PPh₃)₄, 22 g (159.1 mmol) of K₂CO₃, 350 ml of THF, and 170 ml of distilled water were put in a flask and stirred under reflux for 12. hours. After removing a distilled water layer therefrom, methanol was added thereto, producing solids. The solids were filtered, washed with water, methanol, and acetone, and dried, obtaining 32 g (Yield: 85%) of Compound 1-25.

(LC/MS theoretical value 713.83, measured value 714.50)

### Synthesis Example 2: Synthesis of Compound 1-9

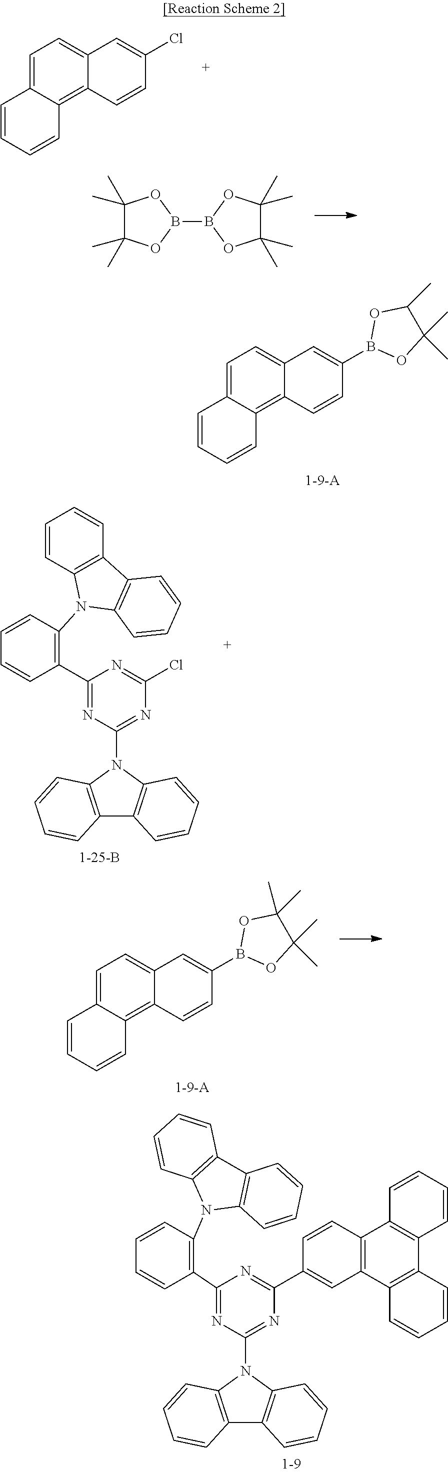

### 1st Step: Synthesis of Intermediate 1-9-A

27.8 g (130.7 mmol) of 2-Chlorophenanthrene, 43.2 g (170 mmol) of bis(pinacolato)diboron, 7.2 g (7.8 mmol) of Pd₂(dba)₃, 8.8 g (31.4 mmol) of tricyclophosphine, 38.5 g (392.1 mmol) of potassium acetate, and 330 ml of xylene were put in a flask and then, stirred under reflux for 12 hours. When a reaction was completed, the resultant was silica gel-filtered and treated to remove a solvent, obtaining Intermediate 1-9-A, which was used in the following reaction.

### 2nd Step: Synthesis of Compound 1-9

12 g (23 mmol) of Intermediate 1-25-B, 11.2 g (27.6 mmol) of Intermediate 1-9-A, 0.8 g (0.7 mmol) of Pd(PPh₃)₄, 9.5 g (69 mmol) of K₂CO₃, 150 ml of THF, and 80 ml of distilled water were put in a flask and stirred under reflux for 12 hours. After removing a distilled water layer therefrom, methanol was added thereto to produce solids. The solids were filtered, washed with water, methanol, and acetone, and dried, obtaining 14 g (Yield: 92%) of Compound 1-9.

(LC/MS theoretical value 663.77, measured value 664.40)

### Synthesis Example 3: Synthesis of Compound B-1

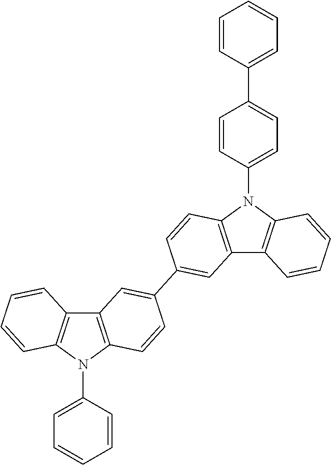 [SMILES: c1ccc(-c2ccc(-n3c4ccccc4c4cc(-c5ccc6c(c5)c5ccccc5n6-c5ccccc5)ccc43)cc2)cc1]

The above Compound B-1 was synthesized with reference to a synthesis method known in U.S. Pat. No. 10,476,008 B2.

### Comparative Synthesis Example 1: Synthesis of Compound Y1

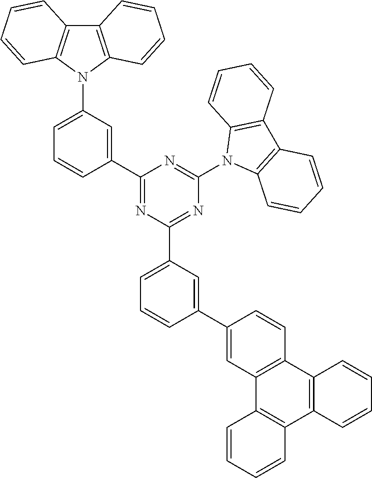 [SMILES: c1cc(-c2ccc3c4ccccc4c4ccccc4c3c2)cc(-c2nc(-c3cccc(-n4c5ccccc5c5ccccc54)c3)nc(-n3c4ccccc4c4ccccc43)n2)c1]

Compound Y1 was synthesized with reference to the synthesis method known in Publication KR 10-2020-0087020.

### Comparative Synthesis Example 2: Synthesis of Compound Y2

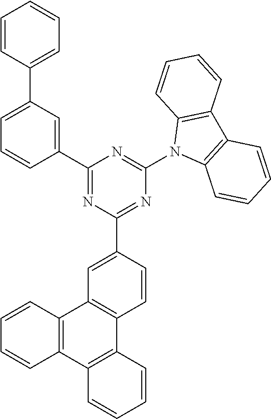 [SMILES: c1ccc(-c2cccc(-c3nc(-c4ccc5c6ccccc6c6ccccc6c5c4)nc(-n4c5ccccc5c5ccccc54)n3)c2)cc1]

Compound Y2 was synthesized with reference to the synthesis method known in Publication KR 10-2019-0090204.

### Comparative Synthesis Example 3: Synthesis of Compound Y3

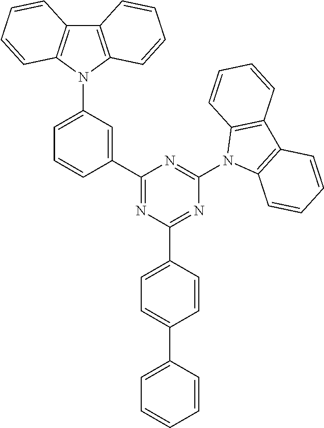 [SMILES: c1ccc(-c2ccc(-c3nc(-c4cccc(-n5c6ccccc6c6ccccc65)c4)nc(-n4c5ccccc5c5ccccc54)n3)cc2)cc1]

Compound Y3 was synthesized with reference to the synthesis method known in Publication KR 10-2020-0087020.

(Manufacture of Organic Light Emitting Diode)

### Example 1

Referring to the compounds below, a glass substrate coated with a thin film of indium tin oxide (ITO) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. The thus obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1350 Å-thick hole transport layer. On the hole transport layer, Compound B was deposited at a thickness of 350 Å to form a hole transport auxiliary layer. On the hole transport auxiliary layer, a 400 Å-thick light emitting layer was formed by using Compound 1-25 obtained in Synthesis Example 1 and doping 7 wt % of PhGD as a dopant by vacuum-deposition. Subsequently, on the light emitting layer, Compound C was deposited at a thickness of 50 Å to form an electron transport auxiliary layer and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1200 Å-thick, manufacturing an organic light emitting diode.

The organic light emitting diode has a structure of ITO/Compound A (3% NDP-9 doping, 100 Å)/Compound A (1350 Å)/Compound B (350 Å)/EML [93 wt % of host (Compound 1-25): 7 wt % of PhGD] (400 Å)/Compound C (50 Å)/Compound D: LiQ (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-Dibenzofuranyl)phenyl]-N-[4-(9-phenyl-9H-fluoren-9-yl)phenyl][1,1′-biphenyl]-4-amine

Compound C: 2,4-Diphenyl-6-(4′,5′,6′-triphenyl[1,1′:2′,1″:3″,1′″:3′″,1″″-quinquephenyl]-3″″-yl)-1,3,5-triazine

Compound D: 2-(1,1′-Biphenyl-4-yl)-4-(9,9-diphenylfluoren-4-yl)-6-phenyl-1,3,5-triazine

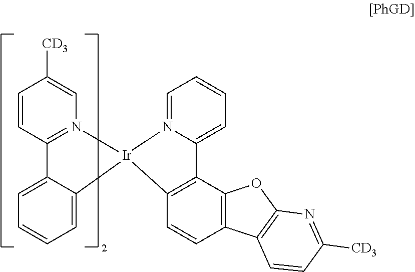

### Example 2

Referring to the compounds below, a glass substrate coated with a thin film of indium tin oxide (ITO) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. The thus obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1350 Å-thick hole transport layer. On the hole transport layer, Compound E was deposited at a thickness of 350 Å to form a hole transport auxiliary layer. On the hole transport auxiliary layer, a 400 Å-thick light emitting layer was formed by using Compound 1-25 obtained in Synthesis Example 1 and Compound B-1 obtained in Synthesis Example 3 simultaneously as a host and doping 10 wt % of PhGD as a dopant by vacuum-deposition. Herein, Compound 1-25 and Compound B-1 were used in a weight ratio of 3:7. Subsequently, Compound F was deposited on the light emitting layer at a thickness of 50 Å to form an electron transport auxiliary layer, and Compound G and LiQ were simultaneously vacuum-deposited at a weight ratio of 1:1 to form a 300 Å-thick electron transport layer. On the electron transport layer, LiQ and Al were sequentially vacuum-deposited to be 15 Å-thick and 1200 Å-thick, manufacturing an organic light emitting diode.

The organic light emitting diode has a structure of ITO/Compound A (3% NDP-9 doping, 100 Å)/Compound A (1350 Å)/Compound E (350 Å)/EML [Compound 1-25: Compound B-1: PhGD=27:63:10 wt %] (400 Å)/Compound F (50 Å)/Compound G: LiQ (300 Å)/LiQ (15 Å)/Al (1200 Å).

Compound E: N-[1,1′-Biphenyl]-4-yl-N-(9,9-dimethyl-9H-fluoren-2-yl)-7,7-dimethyl-7H-benzo[b]fluoreno[3,2-d]furan-1-amine

Compound F: 2-[3′-(9,9-Dimethyl-9H-fluoren-2-yl)[1,1′-biphenyl]-3-yl]-4,6-diphenyl-1,3,5-triazine

Compound G: 2-[4-[4-(4′-Cyano-1,1′-biphenyl-4-yl)-1-naphthyl]phenyl]-4,6-diphenyl-1,3,5-triazine

### Comparative Example 1 and 2

Diodes according to Comparative Examples 1 and 2 were manufactured in the same method as in Example 1 except that the host was changed as shown in Table 1.

### Comparative Example 3

A diode according to Comparative Example 3 was manufactured in the same method as in Example 1 except that the host was changed as shown in Table 2.

Evaluation

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminous efficiency (cd/A) of the same current density (10 mA/cm²) was calculated using the luminance and current density measured from the (1) and (2).

The relative values based on luminous efficiency of Comparative Example 3 are shown in Table 2.

(4) Measurement of Life-Span

T95 life-spans of the manufactured diodes were measured as a time when their luminance decreased down to 95% relative to the initial luminance (cd/m²) after emitting light with 24,000 cd/m² as the initial luminance (cd/m²) and measuring their luminance decreases depending on a time with a Polanonix life-span measurement system.

The relative values based on the T95 life-span of Comparative Example 2 are shown in Table 1, and the relative values based on the T95 life-span of Comparative Example 3 are shown in Table 2.

(5) Measurement of Driving Voltage

The driving voltage of each diode was measured at 15 mA/cm² using a current-voltmeter (Keithley 2400), and the results were obtained.

The relative values based on the driving voltages of Comparative Example 2 were calculated and shown in Table 1.

TABLE 1

| | | Driving | Life-span |
|---|---|---|---|
| | Host | voltage (%) | T95 (%) |
| Example 1 | Compound 1-25 | 86 | 108 |
| Comparative | Comparative Compound Y1 | 93 | 25 |
| Example 1 | | | |
| Comparative | Comparative Compound Y2 | 100 | 100 |
| Example 2 | | | |

TABLE 2

| | | | Luminous | Life- |
|---|---|---|---|---|
| | | | efficiency | span |
| | First host | Second host | (%) | T95 (%) |
| Example 2 | Compound 1-25 | Compound B-1 | 109 | 157 |
| Comparative | Comparative | Compound B-1 | 100 | 100 |
| Example 3 | Compound Y3 | | | |

Referring to Tables 1 and 2, the organic light emitting diodes according to Examples exhibited significantly improved driving, efficiency, and life-span characteristics, compared with the organic light emitting diodes according to the Comparative Examples.

As described above, an example embodiment may provide a compound for an organic optoelectronic device capable of implementing a low-driving, high-efficiency, and long life-span organic optoelectronic device.

An example embodiment provides a composition for an organic optoelectronic device including the compound for the organic optoelectronic device. Another example embodiment provides an organic optoelectronic device including the compound. Another example embodiment provides a display device including the organic optoelectronic device.

According to an example embodiment, a low-driving, high-efficiency, and long life-span organic optoelectronic device may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

### DESCRIPTION OF SYMBOLS

100: organic light emitting diode
105: organic layer
110: cathode
120: anode
130: light emitting layer
140: hole transport region
150: electron transport region

What is claimed is:

1. A compound for an organic optoelectronic device, the compound being represented by Chemical Formula 1: 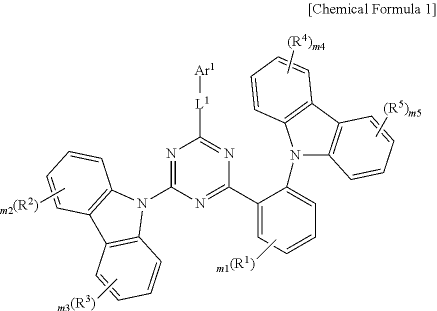
wherein, in Chemical Formula 1,
Ar¹ is a substituted or unsubstituted C10 to C30 condensed aryl group,
L¹ is a single bond or a substituted or unsubstituted C6 to C12 arylene group,
R¹ is hydrogen, deuterium, or an unsubstituted C6 to C12 aryl group,
R² to R⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
m1 to m5 are each independently an integer of 1 to 4.

2. The compound as claimed in claim 1, wherein Ar¹ is a substituted or unsubstituted naphthyl group, a substituted or unsubstituted anthracenyl group, a substituted or unsubstituted phenanthrenyl group, a substituted or unsubstituted triphenylene group, a substituted or unsubstituted pyrenyl group, a substituted or unsubstituted chrysenyl group, a substituted or unsubstituted perylenyl group, a substituted or unsubstituted tetracene, a substituted or unsubstituted benzanthracene, or a combination thereof.

3. The compound as claimed in claim 1, wherein Ar¹ is a substituted or unsubstituted naphthyl group, a substituted or unsubstituted phenanthrenyl group, a substituted or unsubstituted triphenylene group, or a combination thereof.

4. The compound as claimed in claim 1, wherein Ar¹ is one selected from the substituted or unsubstituted groups of Group I: 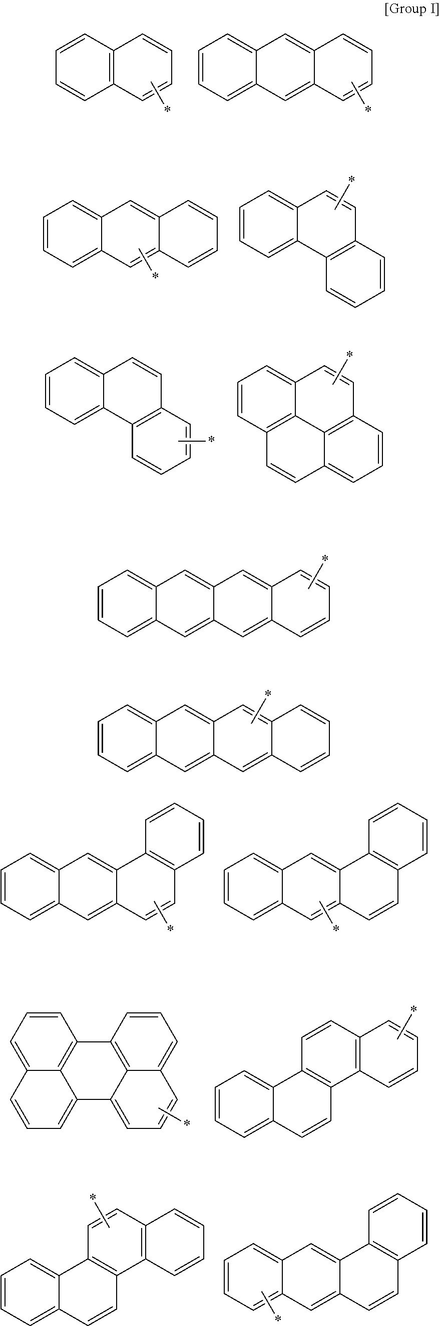 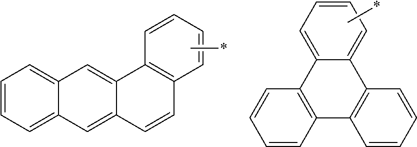
wherein, in Group I, * is a linking point.

5. The compound as claimed in claim 1, wherein the compound is one selected from compounds of Group 1:

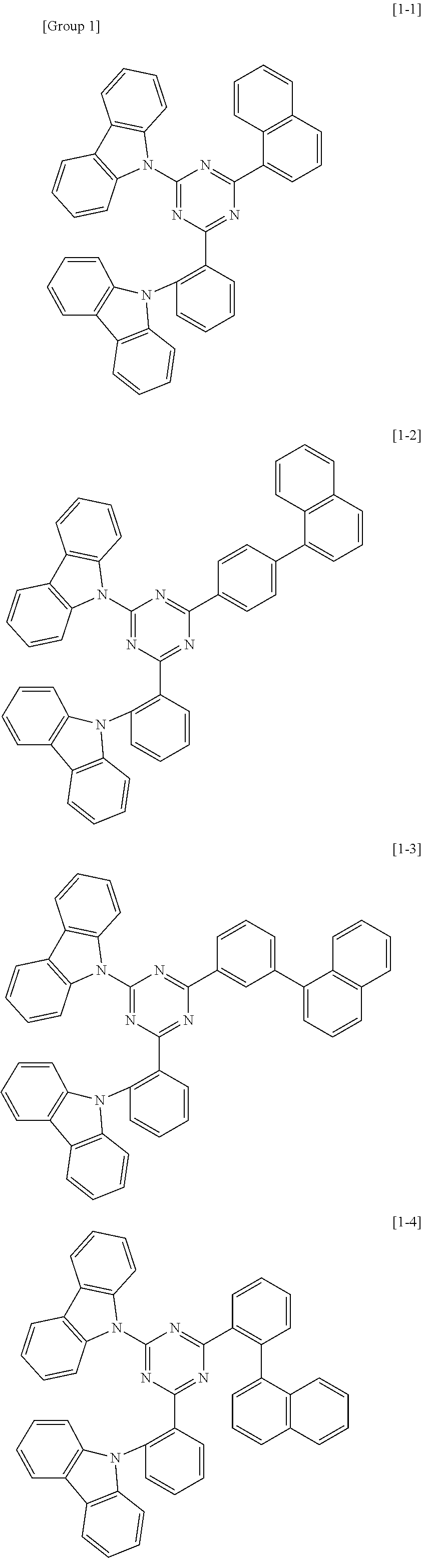

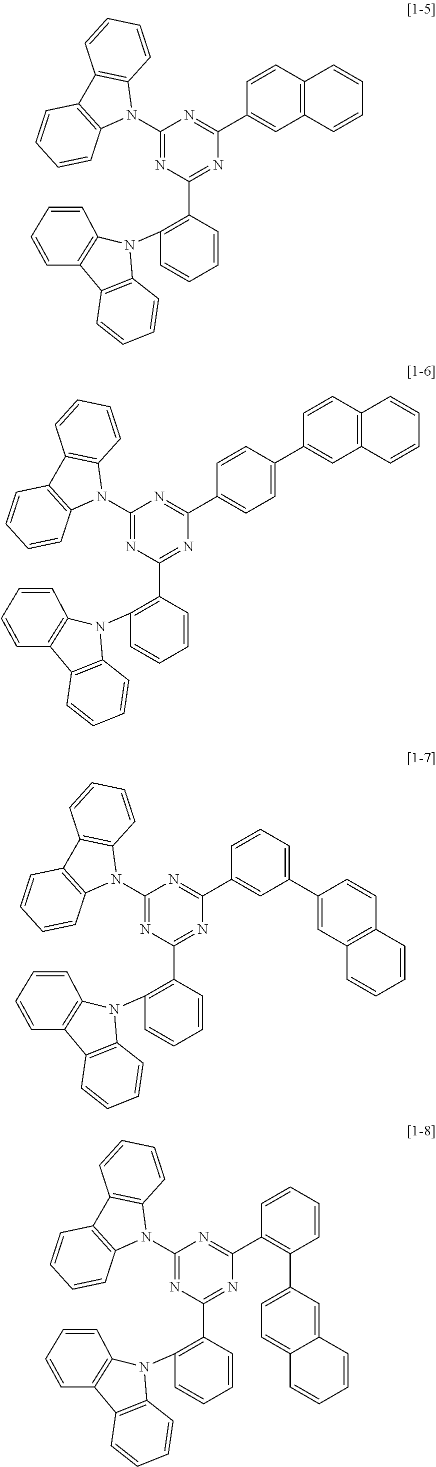

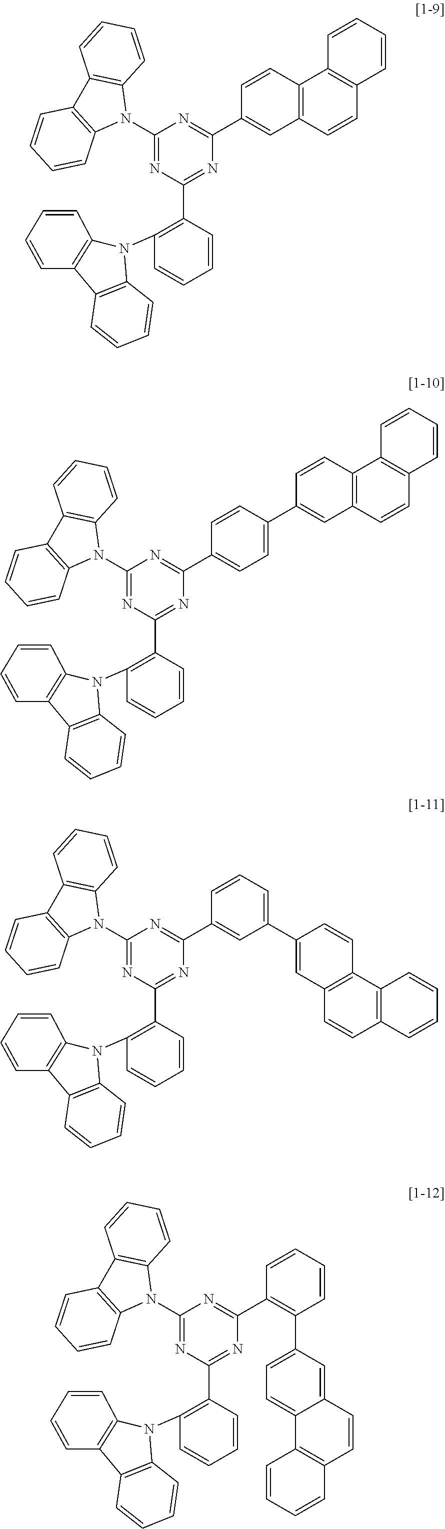 [SMILES: c1ccc(-n2c3ccccc3c3ccccc32)c(-c2nc(-c3ccc4c(ccc5ccccc54)c3)nc(-n3c4ccccc4c4ccccc43)n2)c1 ; c1ccc(-n2c3ccccc3c3ccccc32)c(-c2nc(-c3ccc(-c4ccc5c(ccc6ccccc65)c4)cc3)nc(-n3c4ccccc4c4ccccc43)n2)c1 ; c1cc(-c2ccc3c(ccc4ccccc43)c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c(ccc4ccccc43)c2)c1]

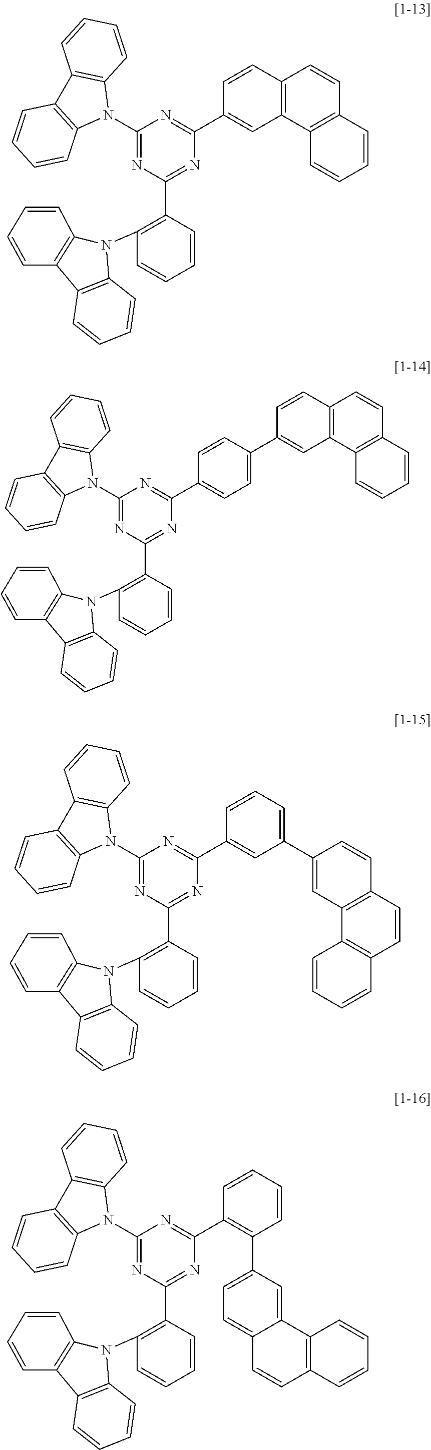 [SMILES: c1ccc(-n2c3ccccc3c3ccccc32)c(-c2nc(-c3ccc4ccc5ccccc5c4c3)nc(-n3c4ccccc4c4ccccc43)n2)c1 ; c1ccc(-n2c3ccccc3c3ccccc32)c(-c2nc(-c3ccc(-c4ccc5ccc6ccccc6c5c4)cc3)nc(-n3c4ccccc4c4ccccc43)n2)c1 ; c1cc(-c2ccc3ccc4ccccc4c3c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3ccc4ccccc4c3c2)c1]

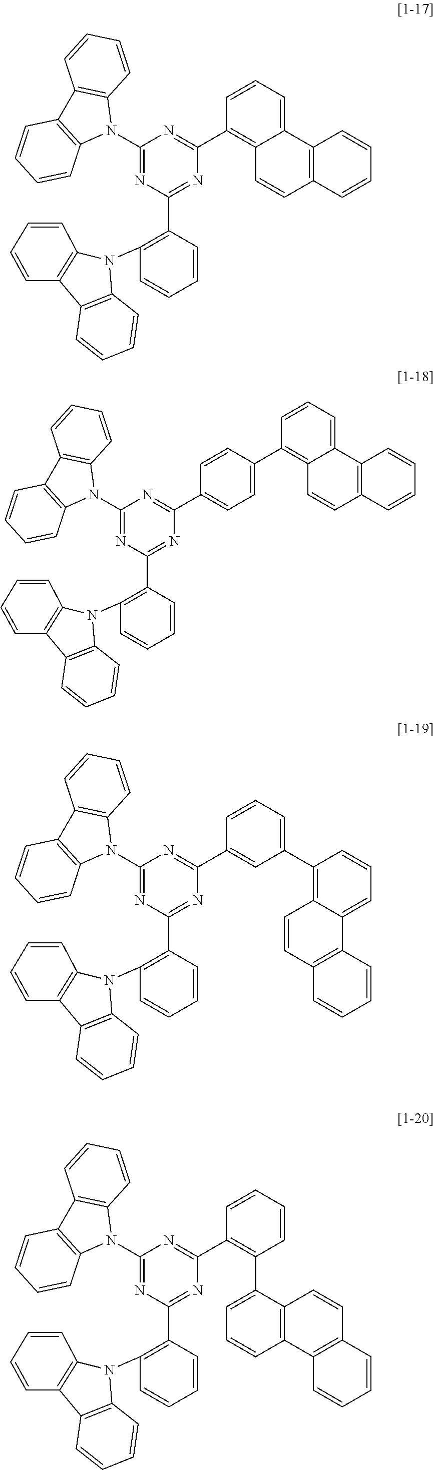 [SMILES: c1ccc(-n2c3ccccc3c3ccccc32)c(-c2nc(-c3cccc4c3ccc3ccccc34)nc(-n3c4ccccc4c4ccccc43)n2)c1 ; c1ccc(-n2c3ccccc3c3ccccc32)c(-c2nc(-c3ccc(-c4cccc5c4ccc4ccccc45)cc3)nc(-n3c4ccccc4c4ccccc43)n2)c1 ; c1cc(-c2nc(-c3ccccc3-n3c4ccccc4c4ccccc43)nc(-n3c4ccccc4c4ccccc43)n2)cc(-c2cccc3c2ccc2ccccc23)c1 ; c1ccc(-c2cccc3c2ccc2ccccc23)c(-c2nc(-c3ccccc3-n3c4ccccc4c4ccccc43)nc(-n3c4ccccc4c4ccccc43)n2)c1]

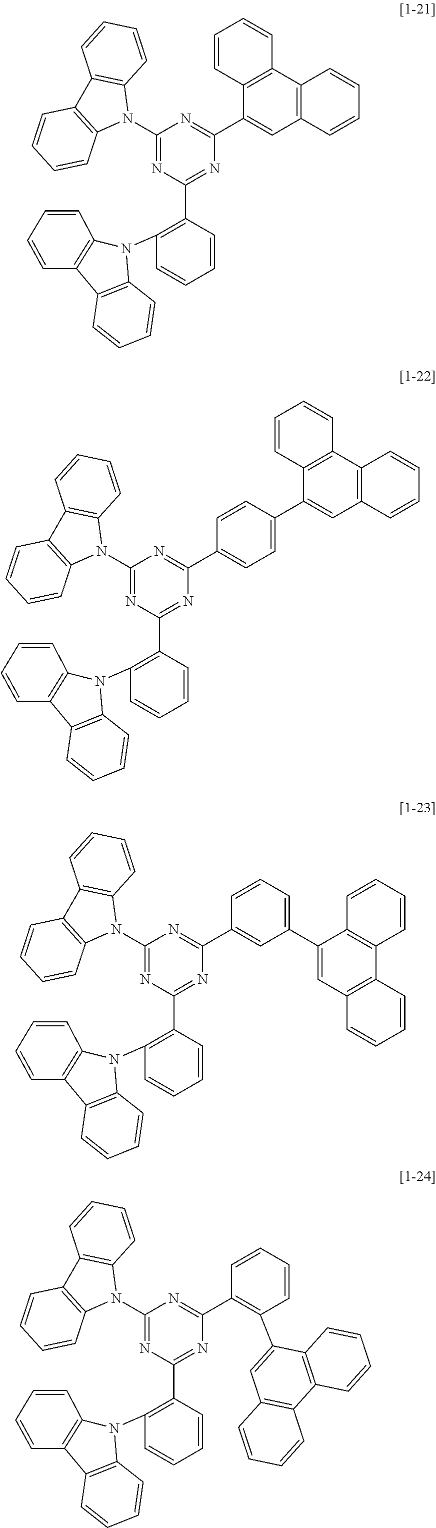

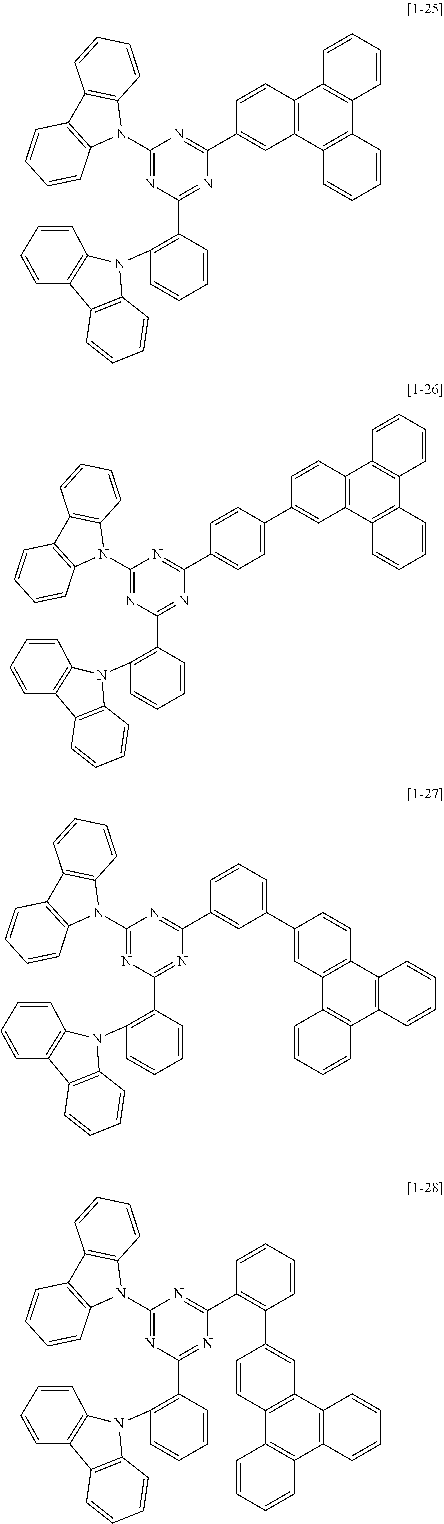 [SMILES: c1ccc(-n2c3ccccc3c3ccccc32)c(-c2nc(-c3ccc4c5ccccc5c5ccccc5c4c3)nc(-n3c4ccccc4c4ccccc43)n2)c1 ; c1ccc(-n2c3ccccc3c3ccccc32)c(-c2nc(-c3ccc(-c4ccc5c6ccccc6c6ccccc6c5c4)cc3)nc(-n3c4ccccc4c4ccccc43)n2)c1 ; c1cc(-c2ccc3c4ccccc4c4ccccc4c3c2)cc(-c2nc(-c3ccccc3-n3c4ccccc4c4ccccc43)nc(-n3c4ccccc4c4ccccc43)n2)c1 ; c1ccc(-c2nc(-c3ccccc3-n3c4ccccc4c4ccccc43)nc(-n3c4ccccc4c4ccccc43)n2)c(-c2ccc3c4ccccc4c4ccccc4c3c2)c1]

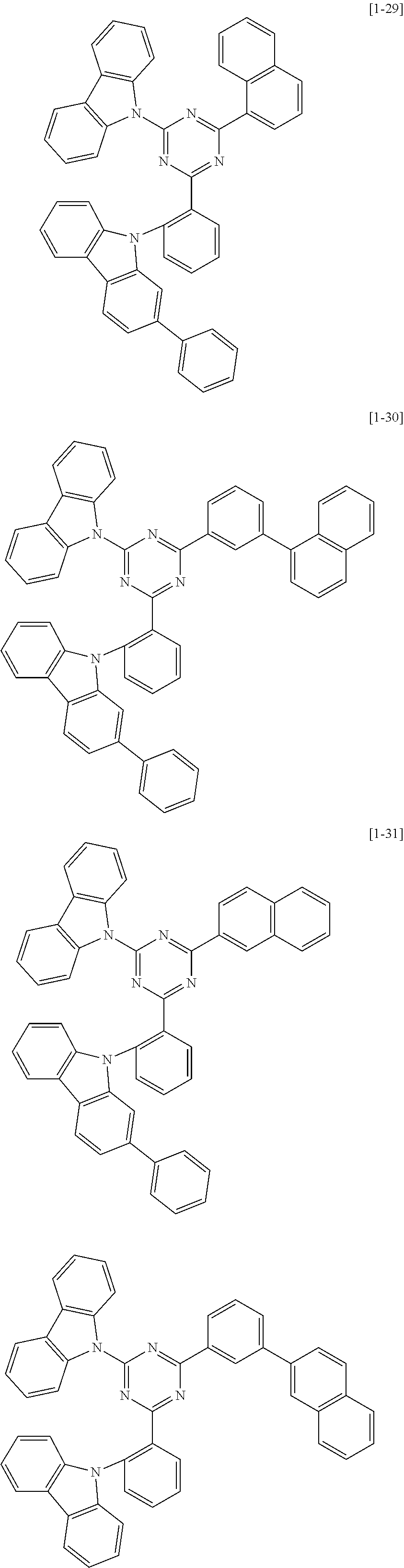 [SMILES: c1ccc(-c2ccc3c4ccccc4n(-c4ccccc4-c4nc(-c5cccc6ccccc56)nc(-n5c6ccccc6c6ccccc65)n4)c3c2)cc1 ; c1ccc(-c2ccc3c4ccccc4n(-c4ccccc4-c4nc(-c5cccc(-c6cccc7ccccc67)c5)nc(-n5c6ccccc6c6ccccc65)n4)c3c2)cc1 ; c1ccc(-c2ccc3c4ccccc4n(-c4ccccc4-c4nc(-c5ccc6ccccc6c5)nc(-n5c6ccccc6c6ccccc65)n4)c3c2)cc1 ; c1cc(-c2ccc3ccccc3c2)cc(-c2nc(-c3ccccc3-n3c4ccccc4c4ccccc43)nc(-n3c4ccccc4c4ccccc43)n2)c1]

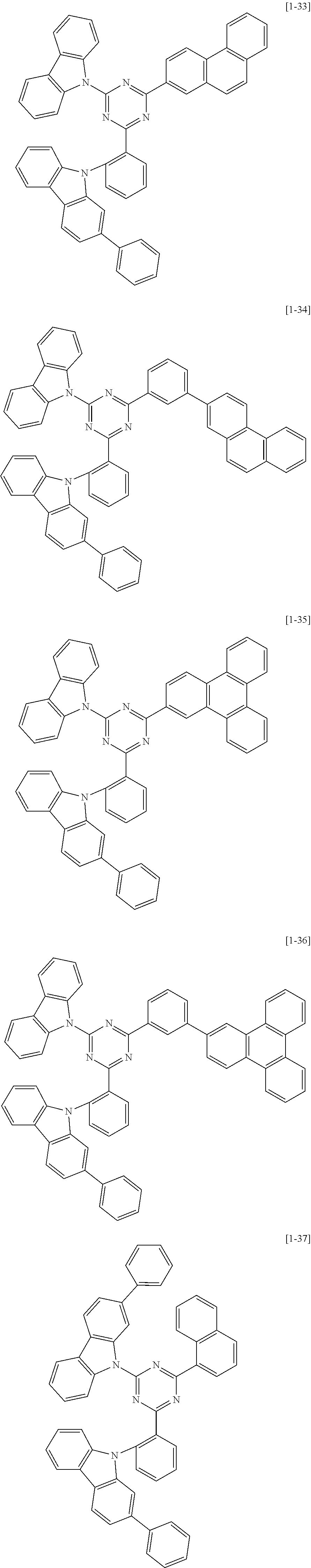

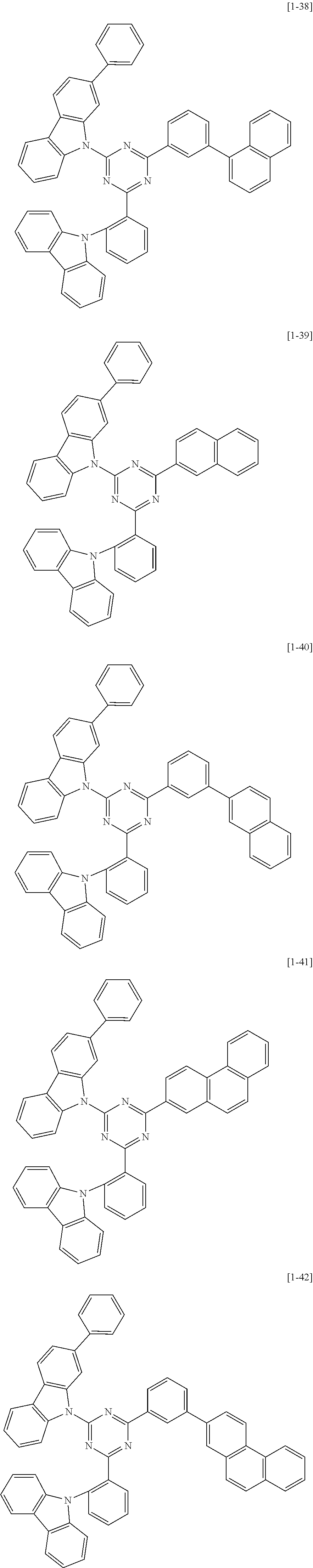

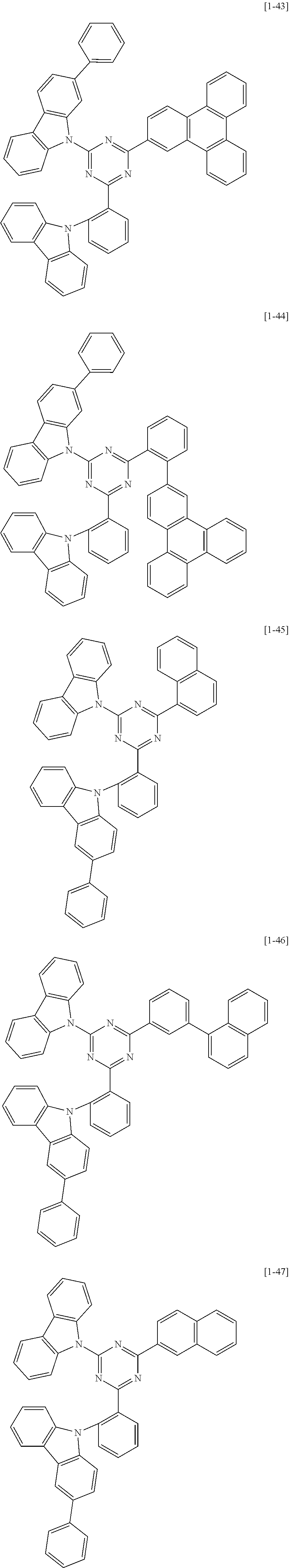

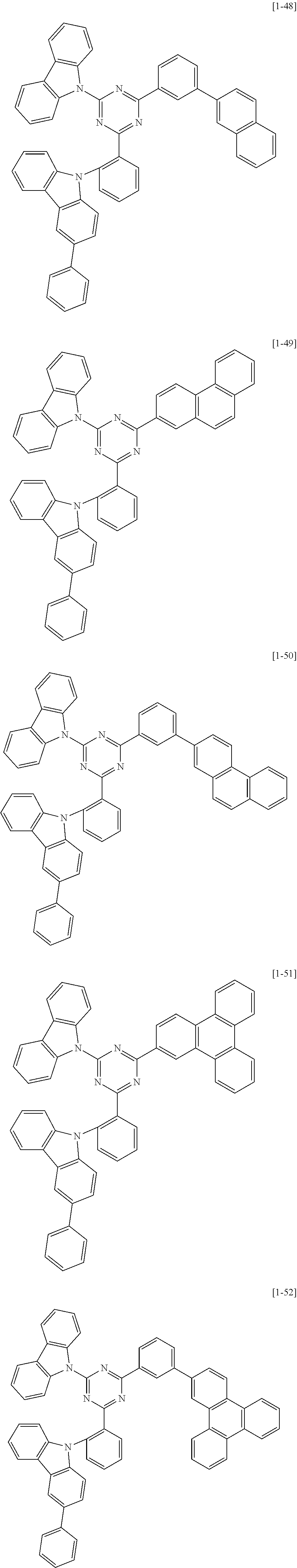

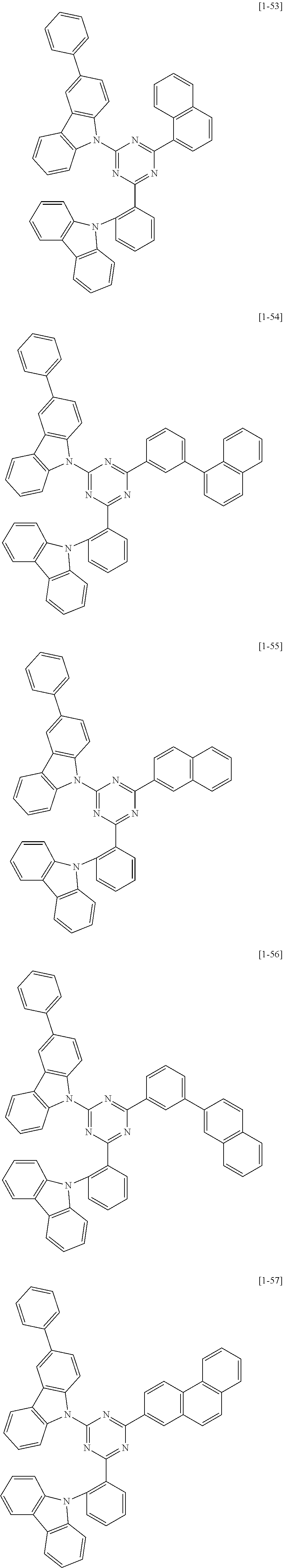 [SMILES: c1ccc(-c2ccc3c(c2)c2ccccc2n3-c2nc(-c3ccccc3-n3c4ccccc4c4ccccc43)nc(-c3cccc4ccccc34)n2)cc1 ; c1ccc(-c2ccc3c(c2)c2ccccc2n3-c2nc(-c3cccc(-c4cccc5ccccc45)c3)nc(-c3ccccc3-n3c4ccccc4c4ccccc43)n2)cc1 ; c1ccc(-c2ccc3c(c2)c2ccccc2n3-c2nc(-c3ccc4ccccc4c3)nc(-c3ccccc3-n3c4ccccc4c4ccccc43)n2)cc1 ; c1ccc(-c2ccc3c(c2)c2ccccc2n3-c2nc(-c3cccc(-c4ccc5ccccc5c4)c3)nc(-c3ccccc3-n3c4ccccc4c4ccccc43)n2)cc1 ; c1ccc(-c2ccc3c(c2)c2ccccc2n3-c2nc(-c3ccc4c(ccc5ccccc54)c3)nc(-c3ccccc3-n3c4ccccc4c4ccccc43)n2)cc1]

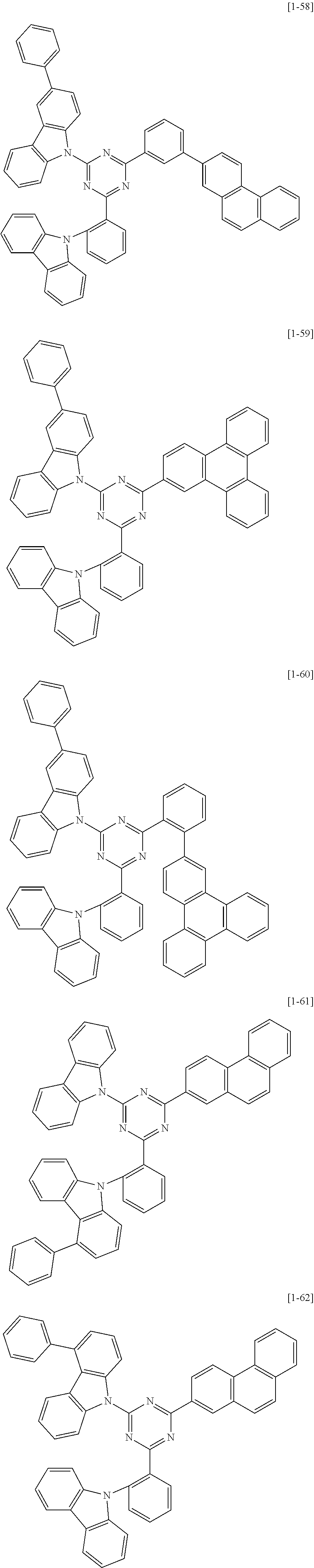

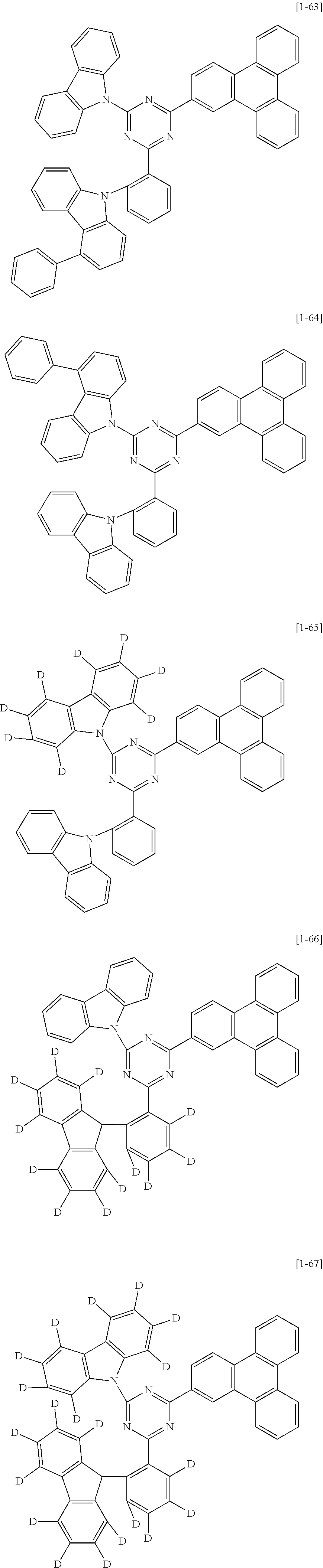

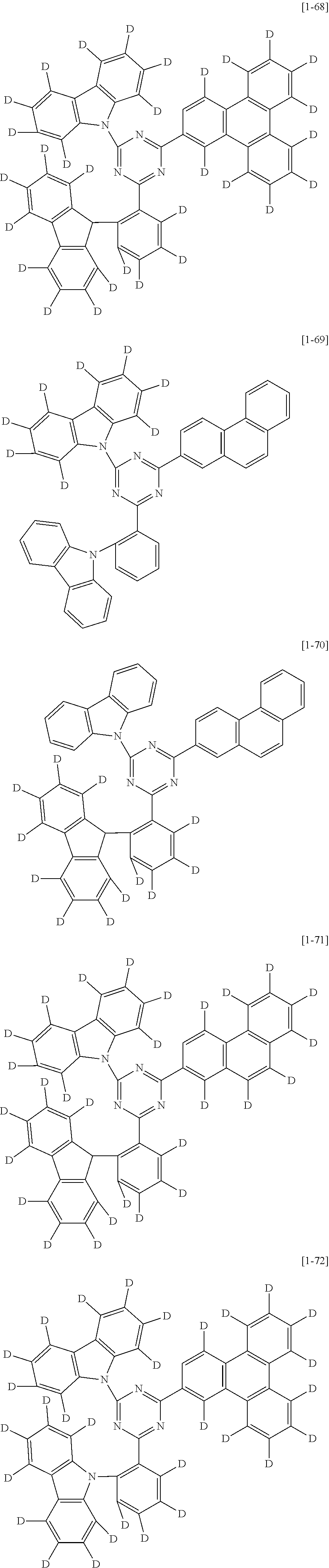

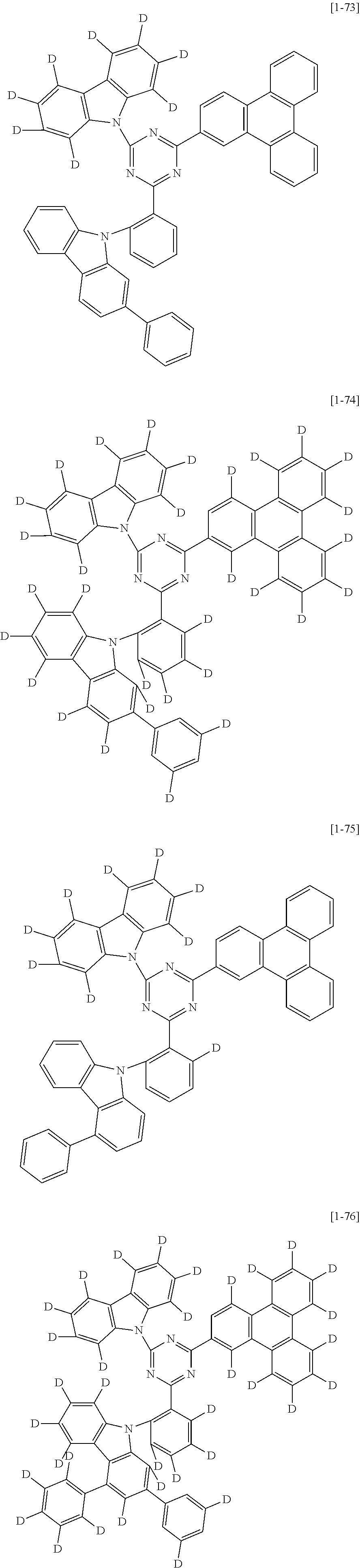

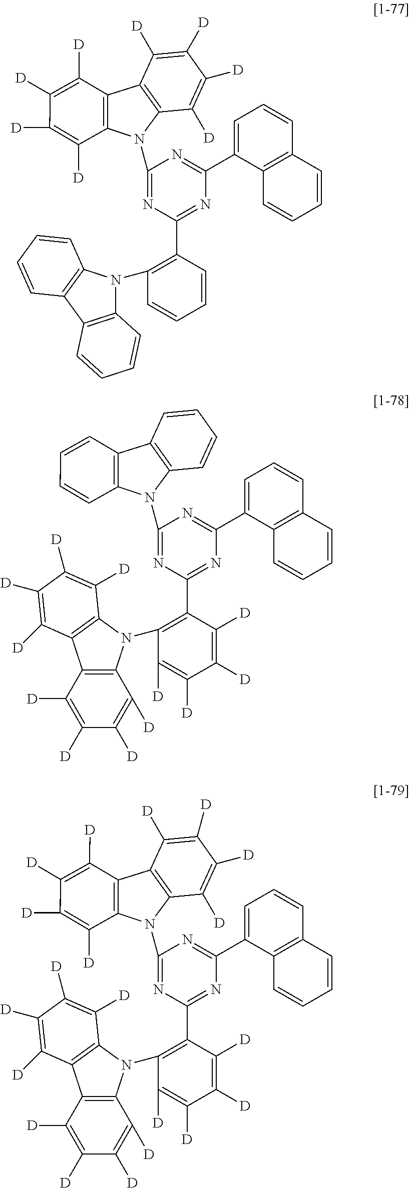 [SMILES: [2H]c1c([2H])c([2H])c2c(c1[2H])c1c([2H])c([2H])c([2H])c([2H])c1n2-c1nc(-c2ccccc2-n2c3ccccc3c3ccccc32)nc(-c2cccc3ccccc23)n1 ; [2H]c1c([2H])c([2H])c(-n2c3c([2H])c([2H])c([2H])c([2H])c3c3c([2H])c([2H])c([2H])c([2H])c32)c(-c2nc(-c3cccc4ccccc34)nc(-n3c4ccccc4c4ccccc43)n2)c1[2H] ; [2H]c1c([2H])c([2H])c(-n2c3c([2H])c([2H])c([2H])c([2H])c3c3c([2H])c([2H])c([2H])c([2H])c32)c(-c2nc(-c3cccc4ccccc34)nc(-n3c4c([2H])c([2H])c([2H])c([2H])c4c4c([2H])c([2H])c([2H])c([2H])c43)n2)c1[2H]]

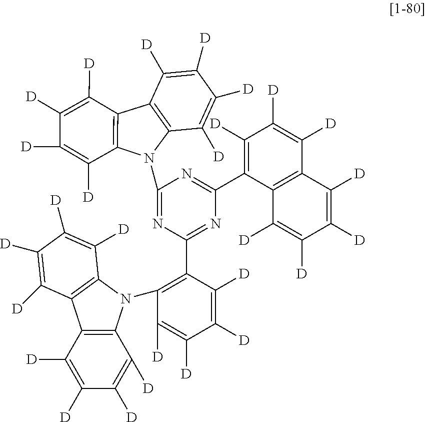 [SMILES: [2H]c1c([2H])c([2H])c(-n2c3c([2H])c([2H])c([2H])c([2H])c3c3c([2H])c([2H])c([2H])c([2H])c32)c(-c2nc(-c3c([2H])c([2H])c([2H])c4c([2H])c([2H])c([2H])c([2H])c34)nc(-n3c4c([2H])c([2H])c([2H])c([2H])c4c4c([2H])c([2H])c([2H])c([2H])c43)n2)c1[2H]]

6. A composition for an organic optoelectronic device, comprising:
a first compound and a second compound,
wherein the first compound is the compound as claimed in claim 1, and
the second compound is a compound represented by Chemical Formula 2; or a compound represented by a combination of Chemical Formulas 3 and 4: 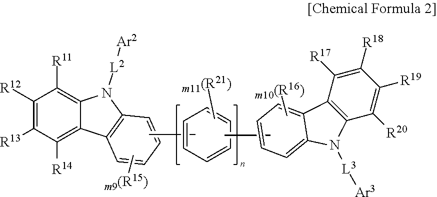
wherein, in Chemical Formula 2,
Ar² and Ar³ are each independently a substituted or unsubstituted C6 to C20 aryl group or a substituted or unsubstituted C2 to C30 heterocyclic group,
L² and L³ are each independently a single bond or a substituted or unsubstituted C6 to C20 arylene group,
R¹¹ to R²¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m9 and m10 are each independently an integer of 1 to 3,
m11 is an integer of 1 to 4, and
n is an integer of 0 to 2; 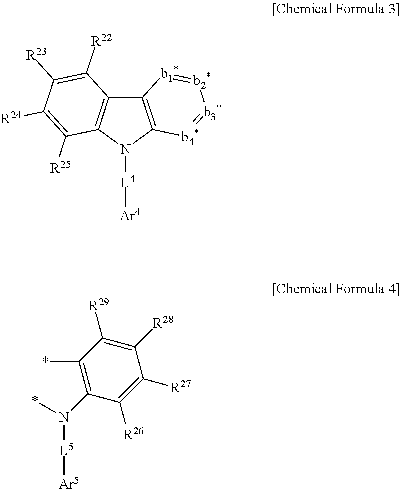
wherein, in Chemical Formulas 3 and 4,
Ar⁴ and Ar⁵ are each independently a substituted or unsubstituted C6 to C20 aryl group or a substituted or unsubstituted C2 to C30 heterocyclic group,
adjacent two of b₁* to b₄* of Chemical Formula 3 are each a carbon (C) linked to * of Chemical Formula 4,
of b₁* to b₄* in Chemical Formula 3, the other two not linked to * in Chemical Formula 4 are each independently C-L^{a}-R^{a},
L^{a}, L⁴, and L⁵ are each independently a single bond or a substituted or unsubstituted C6 to C20 arylene group, and
R^{a} and R²² to R²⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

7. The composition as claimed in claim 6, wherein the second compound is represented by Chemical Formula 2, and Chemical Formula 2 is represented by Chemical Formula 2-8: 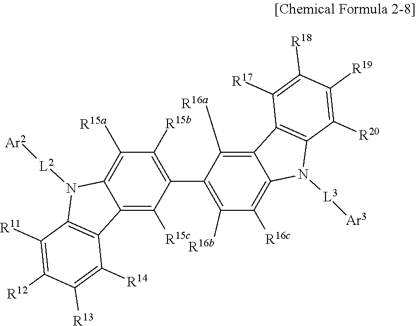
wherein, in Chemical Formula 2-8,
R¹¹ to R¹⁴, R^{15a}, R^{15b}, R^{15c}, R^{16a}, R^{16b}, R^{16c}, and R¹⁷ to R²⁰ are each independently hydrogen, deuterium, or a substituted or unsubstituted C6 to C12 aryl group, and
*-L²-Ar² and *-L³-Ar³ are each independently one of substituents of Group II, 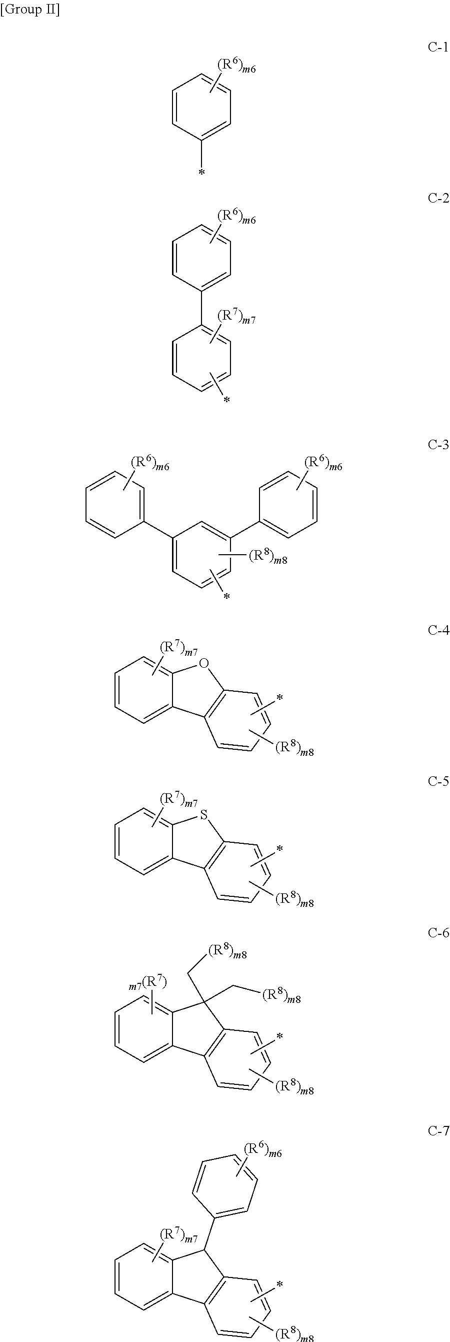 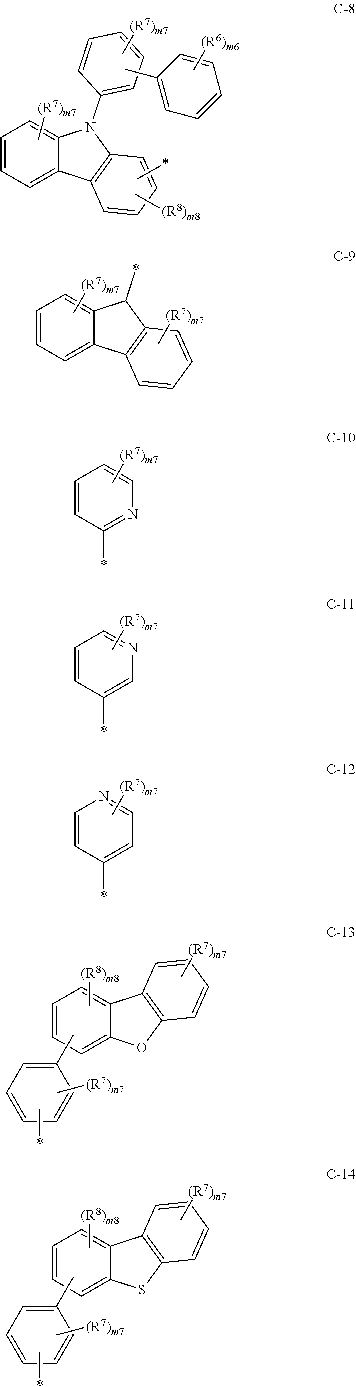 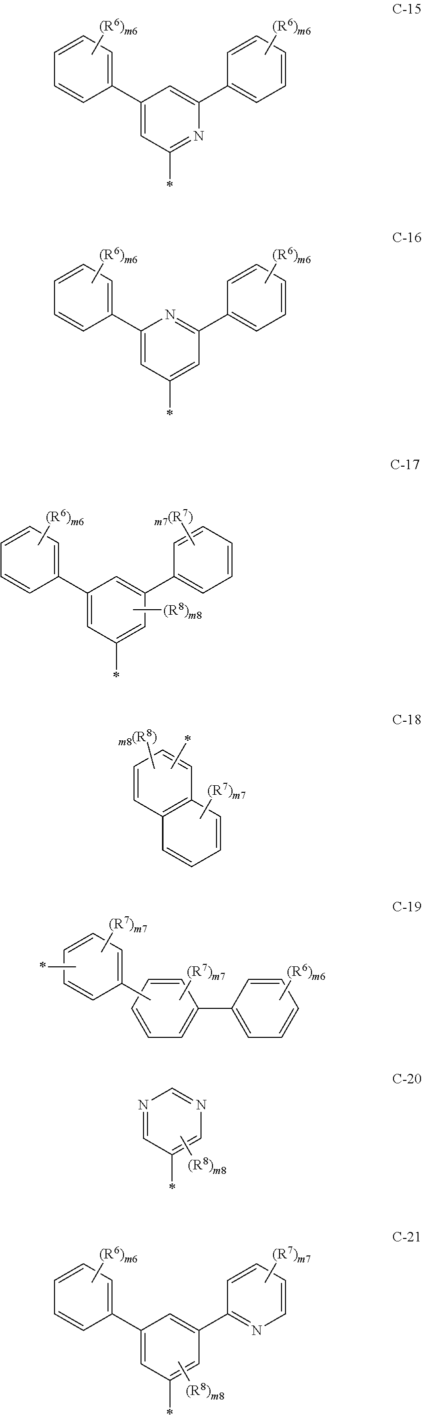 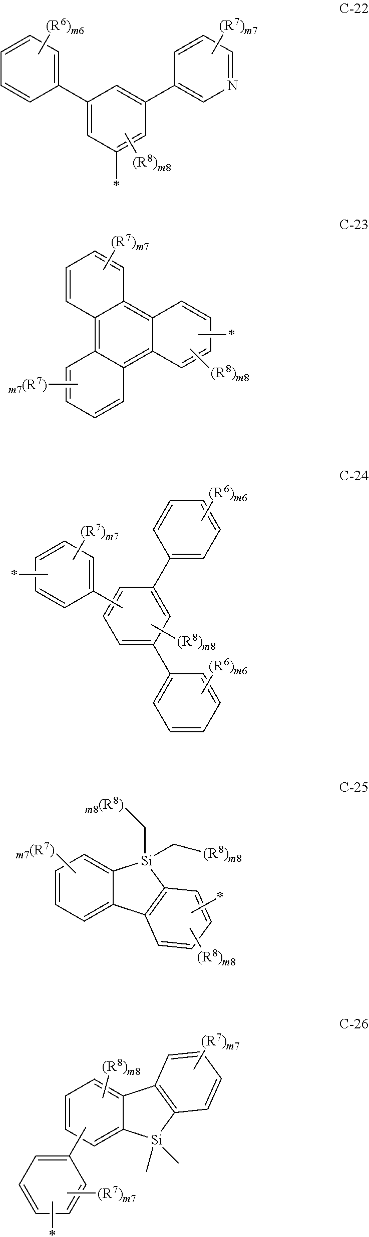
wherein, in Group II,
R⁶ to R⁸ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m6 is an integer of 1 to 5,
m7 is an integer of 1 to 4,
m8 is an integer of 1 to 3, and
* is a linking point.

8. The composition as claimed in claim 6, wherein the second compound is represented by the combination of Chemical Formula 3 and Chemical Formula 4, and the combination of Chemical Formula 3 and Chemical Formula 4 is represented by Chemical Formula 3C: 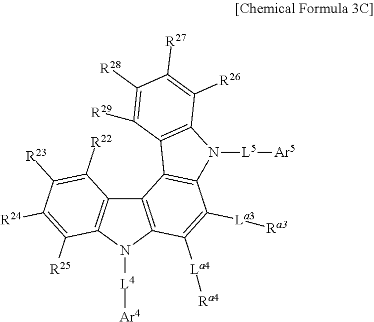
wherein, in Chemical Formula 3C,
L^{a3} and L^{a4} are each a single bond,
R²² to R²⁹, R^{a3}, and R^{a4} are each independently hydrogen, deuterium, or a substituted or unsubstituted C6 to C12 aryl group, and
*-L⁴-Ar⁴ and *-L⁵-Ar⁵ are each independently one of substituents of Group II, 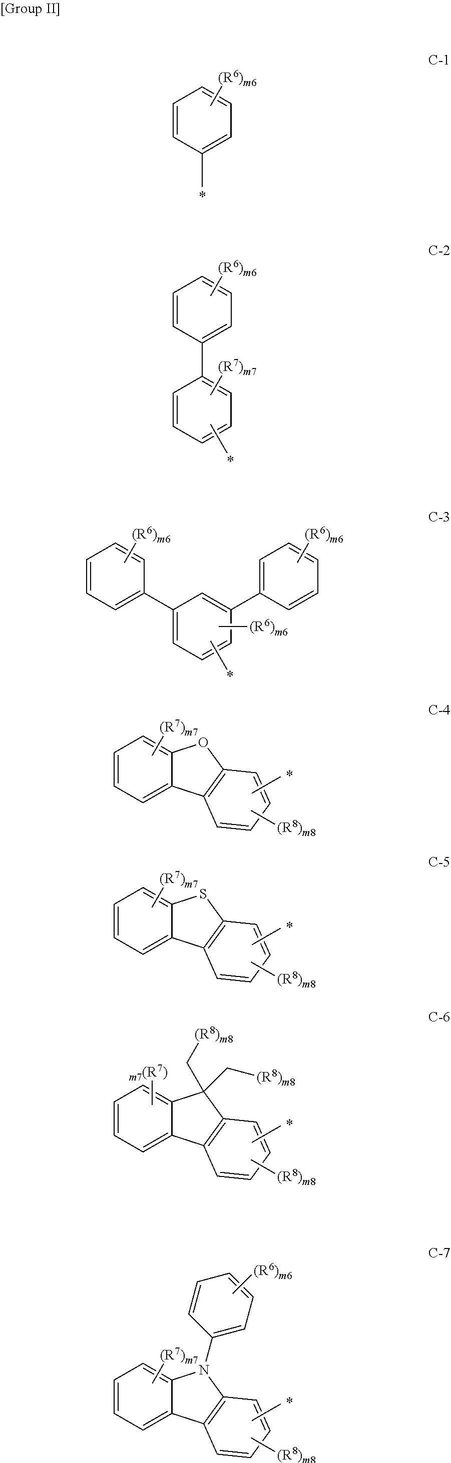 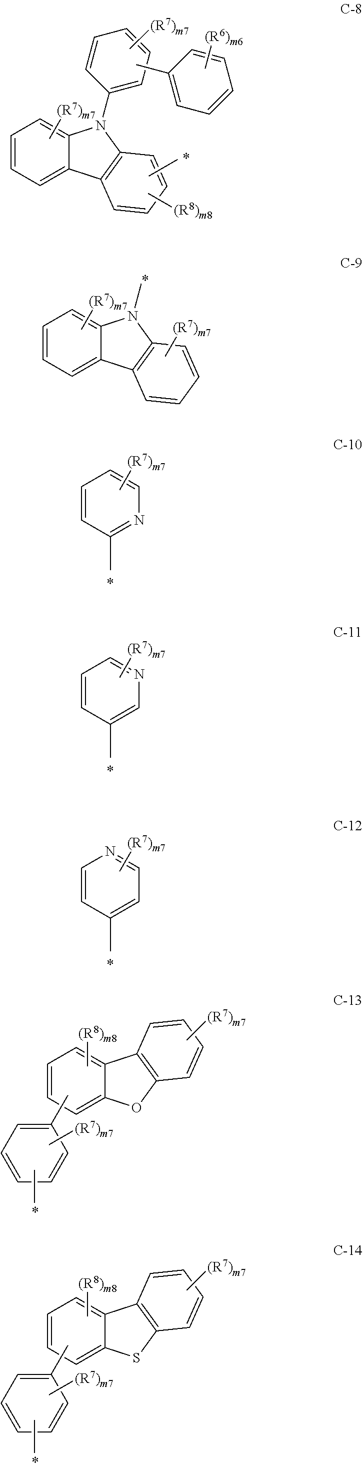 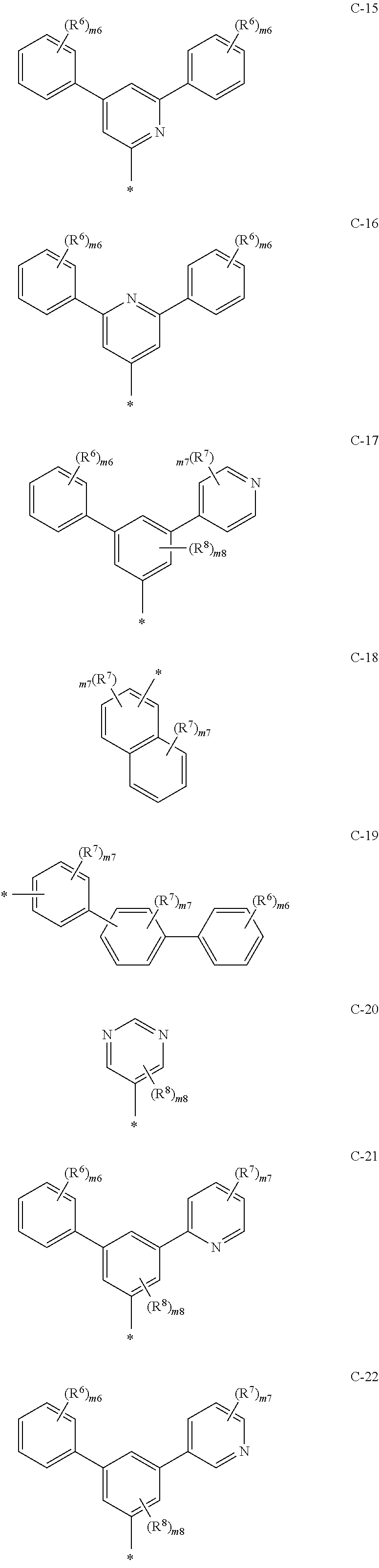 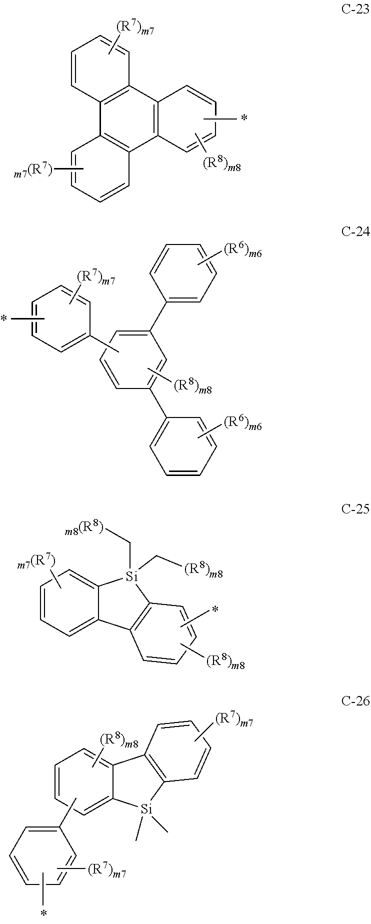
wherein, in Group II,
R⁶ to R⁸ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m6 is an integer of 1 to 5,
m7 is an integer of 1 to 4,
m8 is an integer of 1 to 3, and
* is a linking point.

9. An organic optoelectronic device, comprising:
an anode and a cathode facing each other, and
at least one organic layer between the anode and the cathode,
wherein the at least one organic layer includes the compound for the organic optoelectronic device as claimed in claim 1.

10. The organic optoelectronic device as claimed in claim 9, wherein:
the at least one organic layer includes a light emitting layer, and
the light emitting layer includes the compound for the organic optoelectronic device.

11. A display device comprising the organic optoelectronic device as claimed in claim 9.

12. An organic optoelectronic device, comprising:
an anode and a cathode facing each other, and
at least one organic layer between the anode and the cathode,
wherein the at least one organic layer includes the composition for the organic optoelectronic device as claimed in claim 6.

13. The organic optoelectronic device as claimed in claim 12, wherein:
the at least one organic layer includes a light emitting layer, and
the light emitting layer includes the composition for the organic optoelectronic device.

14. A display device comprising the organic optoelectronic device as claimed in claim 12.
